(19) United States
(12) Patent Application Publication
(10) Pub. No.: US 2025/0312341 A1
(43) Pub. Date: Oct. 9, 2025

(54) METHODS FOR TREATING PATIENTS WITH LOCALLY ADVANCED AND/OR METASTATIC SOLID TUMORS USING A DGK ZETA INHIBITOR

(71) Applicant: ASTELLAS PHARMA INC., Chuo-ku, Tokyo (JP)

(72) Inventor: Masaomi TAKIZAWA, Tokyo (JP)

(21) Appl. No.: 18/875,807

(22) PCT Filed: Jun. 19, 2023
(86) PCT No.: PCT/IB2023/056335

§ 371 Date: Dec. 17, 2024

Related U.S. Application Data
(60) Provisional application No. 63/353,789, filed on Jun. 20, 2022.

(51) Int. Cl. A61K 31/501; A61P 35/00

(52) U.S. Cl. CPC A61K 31/501; A61P 35/00

(57) ABSTRACT

Some embodiments relate to methods for treating a patient having an advanced and/or metastatic tumor, wherein the patient is treated with an effective amount of at least one diacylgycerol kinase zeta (DGKζ) inhibitor and/or a pharmaceutical composition comprising same. In some embodiments, the advanced and/or metastatic tumor is chosen from melanoma and non-small cell lung cancer (NSCLC).

### CROSS-REFERENCE TO RELATED APPLICATIONS

This application is a national phase filing under 35 C.F.R. § 371 of and claims priority to PCT Patent Application No. PCT/IB2023/056335, filed on Jun. 19, 2023, which claims the benefit of priority of U.S. Provisional Patent Application No. 63/353,789, filed on Jun. 20, 2022, the contents of which are hereby incorporated in their entireties by reference herein.

### BACKGROUND

Cancer is among the leading causes of death in the US, with an estimated 1.7 million new cases and approximately 609,000 deaths in the US in 2018 [National Cancer Institute, 2019]. New therapeutic strategies for the treatment of cancer harness the body's own immune system to mount an antitumor response. However, endogenous immune responses are frequently unable to inhibit tumor growth. This deficiency appears to be due to the immunosuppressive nature of the tumor microenvironment (TME). Tumor-infiltrating lymphocytes (TILs) become ‘exhausted’ or suppressed in the context of multiple signals in the TME resulting in significantly impaired proliferative capacity and effector function [Lantis et al., 2017].

Cancer immunotherapies targeting immune checkpoints have been transformative in the treatment practices of oncology. However, only a subset of all patients in most cancer types effectively responds to these therapies. For example, in advanced-stage melanoma of the skin, the overall survival (OS) has improved dramatically over the last decade from approximately nine months before 2011 to at least two years in 2017 [Luke et al., 2017]. However, approximately 60% to 70% of patients that receive anti-programmed cell death protein 1 (anti-PD-1) therapy (e.g., ipilimumab) or cytotoxic T-lymphocyte antigen-4 (CTLA4) inhibitors (e.g., nivolumab and pembrolizumab) do not respond to treatment. Furthermore, acquired resistance is common, causing some patients who initially responded to the therapy to later experience disease progression. Similarly, even though immunotherapies have improved survival in non-small cell lung cancer (NSCLC), tumors often acquire resistance and the disease progression patterns beyond immunotherapy resistance are not completely understood. As such, the 5-year survival rate of NSCLC remains low at 24% [American Cancer Society, 2020]. Therefore, there is a high unmet need that persists for immune checkpoint inhibitor resistant cancers.

Diacylglycerol kinase (DGK) is a large enzyme family of 10 mammalian DGK isoenzymes. DGK has several alternative splicing products and its isoforms are implicated in the pathogenesis of a wide variety of cancers. In T cells, DGK inhibits diacylglycerol (DAG)-mediated signals following T cell receptor (TCR) engagement by catalyzing the conversion of DAG to phosphatidic acid (PA) [Zhong et al., 2008]. Even when programmed cell death protein 1 (PD-1) is blocked by anti-PD-1 antibodies, DAG downstream signaling may be partially inactivated by DGK. DAG is generated by TCR stimulation and activates RAS-ERK-AP-1 signaling pathway and PKC/PKD-IKK-NFκB signaling pathway in T cells, resulting in enhanced cytokine production and proliferation.

Upregulation of DGK limits RAS activation, leading T cells to exhaustion states [Zhong et al., 2008]. Therefore, DGK inhibitors have the potential to enhance DAG downstream signaling, leading to T cell activation regardless of the PD-1 signal [Wee et al., 2019]. Data suggest that the DGKζ (zeta) isoform, one of the DGK family members, is a negative regulator of DAG-mediated signaling pathways by enzymatically converting DAG into PA, and a dominant regulator of TCR driven T cell activation. This is consistent with its observed superior anti-tumor immune response [Wee et al., 2019].

In the single cell RNA sequencing analysis, DGKζ is expressed in CD8⁺ T cells in biopsy samples of various tumor types, and DGKζ positive rate is relatively high in melanoma and NSCLC. Additionally, its expression is significantly correlated with T cell exhaustion markers in these tumor types. Therefore, DGKζ is considered to contribute to T cell exhaustion in some tumor types, and DGKζ inhibition may show antitumor efficacy by releasing from T cell exhaustion state.

N-[4-(2-Fluorophenoxy)-2-{(3S)-4-methyl-3-[(methylamino)methyl]piperazin-1-yl}-3-(trifluoromethyl)phenyl]-1-(pyridazin-4-yl)-1H-pyrazole-3-carboxamide (referred to herein as “Compound I”) is a selective small molecule inhibitor of DGKζ. The chemical structure of Compound I is shown below.

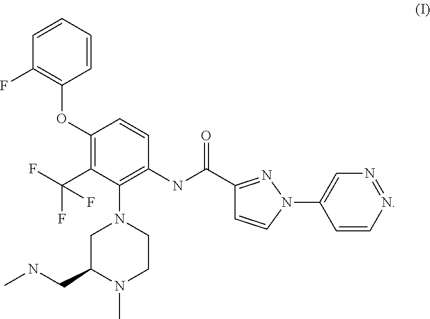 [SMILES: CNC[C@H]1CN(c2c(NC(=O)c3ccn(-c4ccnnc4)n3)ccc(Oc3ccccc3F)c2C(F)(F)F)CCN1C]

Nonclinical pharmacology studies have demonstrated that Compound I inhibits DGKζ activity and enhances activation of DAG downstream pathways as well as T cell activation upon TCR stimulation. A putative mechanism of action is shown in FIG. 1. Further, oral administration of Compound I has demonstrated antitumor efficacy in the inflamed tumor and the TIL-poor tumor models. Further disclosure regarding Compound I, and other small molecule inhibitors of DGKζ suitable for the disclosed methods and compositions, may be found in WO2022114164, published Jun. 2, 2022, which is hereby incorporated by reference.

### SUMMARY OF THE DISCLOSURE

The present disclosure relates to methods for treating a patient having an advanced and/or metastatic tumor (i.e., cancer).

In some embodiments of the methods disclosed herein, the advanced and/or metastatic tumor is chosen from solid tumors. In some embodiments, the solid tumor is chosen from sarcomas, carcinomas, and lymphomas. In some embodiments, the solid tumor is chosen from skin cancer, bladder cancer, breast cancer, uterine cancer, ovary cancer, prostate cancer, lung cancer, colon cancer, pancreas cancer, renal cancer, and gastric cancer.

In some embodiments of the methods disclosed herein, inhibition of DGKζ mediated functions is useful.

In some embodiments of the methods disclosed herein, the advanced and/or metastatic tumor has elevated DGKζ levels.

In some embodiments of the methods disclosed herein, the advanced and/or metastatic tumor is a Tumor-Infiltrating Lymphocyte (TIL) poor tumor.

In some embodiments, the advanced and/or metastatic tumor is an inflamed tumor.

In some embodiments, the advanced and/or metastatic tumor is chosen from melanoma and NSCLC.

In some embodiments, the methods disclosed herein comprise administering to the patient an effective amount of at least one entity chosen from Compound I and pharmaceutically acceptable salts thereof.

In some embodiments, the methods disclosed herein comprise administering to the patient a pharmaceutical composition comprising an effective amount of at least one entity chosen from Compound I and pharmaceutically acceptable salts thereof.

The following detailed description and examples illustrate certain embodiments of the present disclosure. Those of skill in the art will recognize that there are numerous variations and modifications of this disclosure that are encompassed by its scope. Accordingly, the description of certain embodiments should not be deemed to limit the scope of the present disclosure.

All references cited herein, including, but not limited to, published and unpublished applications, patents, and literature references, are incorporated herein by reference in their entirety and are hereby made a part of this specification. To the extent a cited reference conflicts with the disclosure herein, the specification shall control.

### BRIEF DESCRIPTION OF THE DRAWINGS

FIG. 1 is a diagram illustrating a putative mechanism of action of Compound I in T cells.

FIG. 2 illustrates a study design scheme as described in the present disclosure.

FIG. 3 illustrates a study visit scheme as described in the present disclosure.

FIG. 4 illustrates a schedule of assessment as described in the present disclosure.

FIG. 5 illustrates a sample collection schedule as described in the present disclosure.

FIG. 6 illustrates an alternative schedule of assessment in response to crises.

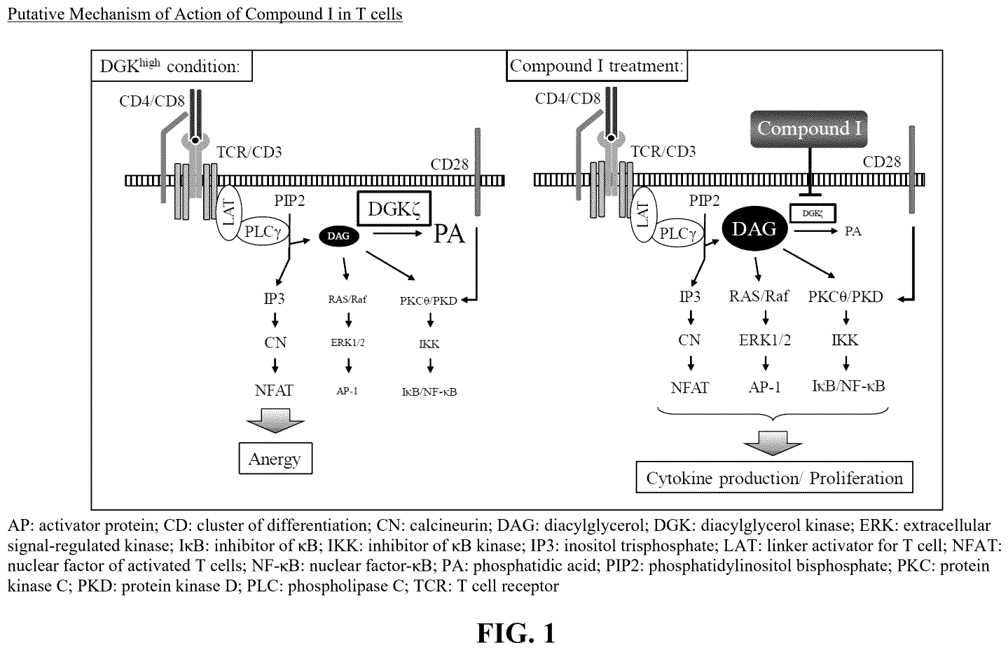

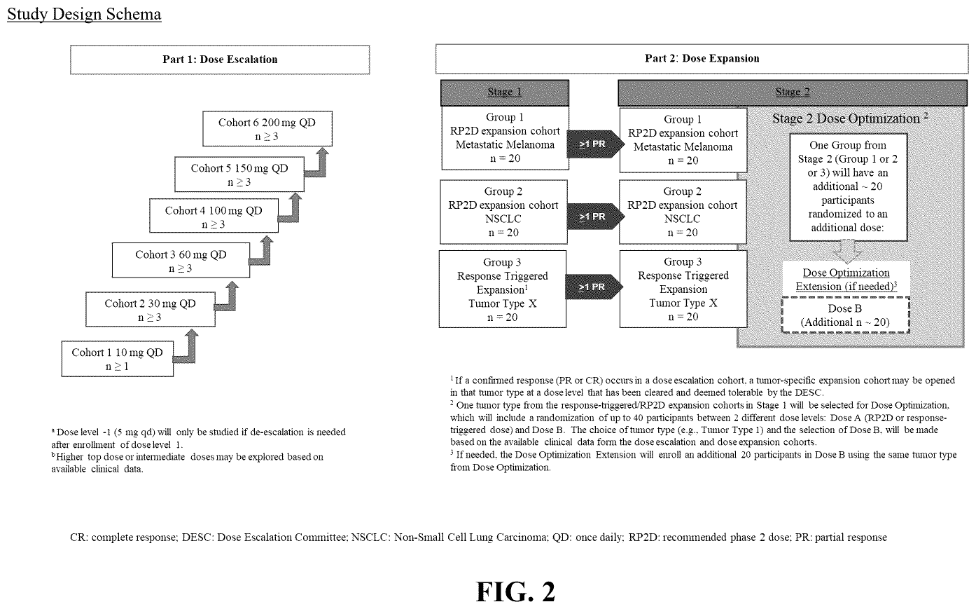

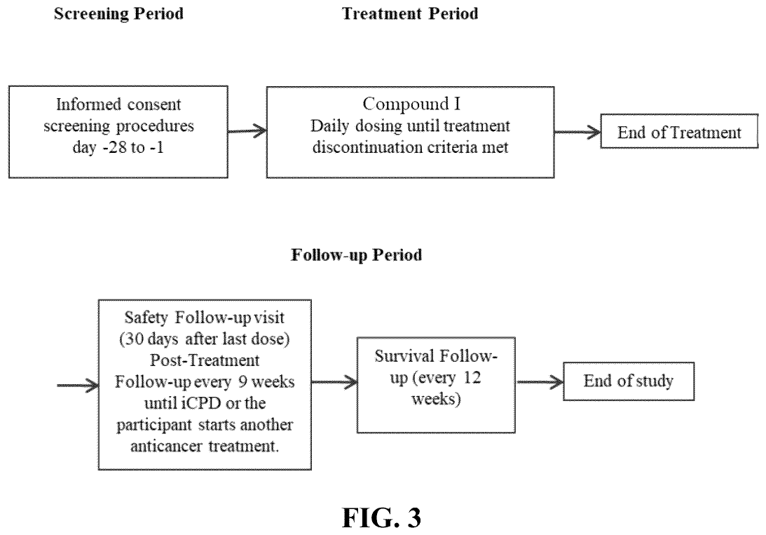

### DETAILED DESCRIPTION OF THE DISCLOSURE

The present disclosure relates to methods for treating a patient having an advanced and/or metastatic tumor.

In some embodiments, the methods disclosed herein comprise administering to the patient an effective amount of at least one entity chosen from Compound I

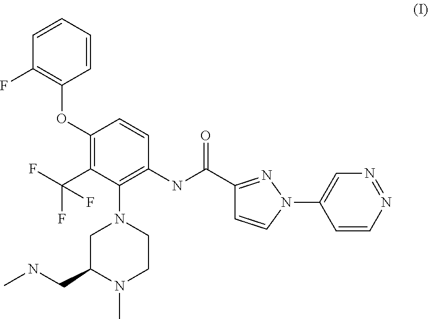 [SMILES: CNC[C@H]1CN(c2c(NC(=O)c3ccn(-c4ccnnc4)n3)ccc(Oc3ccccc3F)c2C(F)(F)F)CCN1C]

and pharmaceutically acceptable salts thereof.

In some embodiments, the methods disclosed herein comprise administering to the patient a pharmaceutical composition comprising an effective amount of at least one entity chosen from Compound I and pharmaceutically acceptable salts thereof.

In some embodiments, the methods disclosed herein comprise orally administering to the patient (1) the at least one entity chosen from Compound I and pharmaceutically acceptable salts thereof and/or (2) the pharmaceutical composition comprising same.

In some embodiments of the methods disclosed herein, the at least one entity is in the form of a pharmaceutically acceptable solvate, mixed solvate, or complex. In some embodiments, the at least one entity is in the form of a non-crystalline solid. In some embodiments, the at least one entity is in the form of a crystalline solid.

In some embodiments of the methods disclosed herein, the at least one entity is Compound I. In some embodiments, the at least one entity is chosen from pharmaceutically acceptable salts of Compound I. Non-limiting examples of pharmaceutically acceptable salts include acid addition salts with inorganic acids such as hydrochloric acid, hydrobromic acid, hydroiodic acid, sulfuric acid, nitric acid and phosphoric acid, and organic acids such as formic acid, acetic acid, propionic acid, oxalic acid, malonic acid, succinic acid, fumaric acid, maleic acid, lactic acid, malic acid, mandelic acid, tartaric acid, dibenzoyltartaric acid, ditoluoyltartaric acid, citric acid, methanesulfonic acid, ethanesulfonic acid, benzenesulfonic acid, p-toluenesulfonic acid, aspartic acid and glutamic acid. Pharmaceutically acceptable salts may, for example, be obtained using standard procedures well known in the field of pharmaceuticals.

In some embodiments of the methods disclosed herein, the at least one entity is in the form of a pharmaceutically acceptable salt of Compound I chosen from HCl, mesylate, succinate, L-malate, L-tartrate, and fumarate salts. In some embodiments, the at least one entity is in the form of an HCl salt. In some embodiments, the at least one entity is in the form of an mesylate salt. In some embodiments, the at least one entity is in the form of a succinate salt. In some embodiments, the at least one entity is in the form of an L-malate salt. In some embodiments, the at least one entity is in the form of an L-tartrate salt. In some embodiments, the at least one entity is in the form of an fumarate salt. In some embodiments, the at least one entity is in the form of a mono-succinate salt. In some embodiments, the at least one entity is in the form of hemi-succinate salt. In some embodiments, the at least one entity is in the form of a mono-L-malate salt. In some embodiments, the at least one entity is in the form of a hemi-L-malate salt.

As used herein, the singular forms of a word also include the plural form, unless the context clearly dictates otherwise; as examples, the terms “a,” “an,” and “the” are understood to be singular or plural. By way of example, “an element” means one or more element.

The terms “at least one” and “one or more” are intended to be synonymous and to refer to no less than one but possibly more, such as one, two, three, etc. For example, the term “at least one entity” refers to one or more entities, such as one entity, two entities, etc.

The term “or” is used herein to mean, and is used interchangeably with, the term “and/or,” unless context clearly indicates otherwise. The term “and/or” as used in a phrase such as “A and/or B” herein is intended to include the following embodiments: “A and B,” “A or B,” “A,” and “B.”

Likewise, the term “and/or” as used in a phrase such as “A, B, and/or C” is intended to encompass each of the following embodiments: “A, B, and C;” “A, B, or C;” “A or C;” “A or B;” “B or C;” “A and C;” “A and B;” “B and C;” “A” (alone); “B” (alone); and “C” (alone).

The terms “treating” or “treatment” or “to treat” refer to therapeutic measures (e.g., administration of a medicament(s) to a patient) that cure, slow down, lessen symptoms of, and/or halt progression of the condition. Treatment need not result in a complete cure of the condition; partial inhibition or reduction of the condition being treated is encompassed by this term.

The terms “patient” and “subject” are used synonymously to refer to an adult human individual.

An “effective amount” refers to an amount of at least one entity of the present disclosure or a pharmaceutical composition comprising at least one such entity of the present disclosure that, when administered to a patient, either as a single dose or as part of a series of doses, is effective to produce at least one therapeutic effect. The dose may depend upon the body mass, weight, and/or blood volume of the patient. Patients may generally be monitored for therapeutic effectiveness using assays suitable for condition being treated. The level of a compound that is administered to a patient may be monitored by determining the level of the compound (or a metabolite of the compound) in a biological fluid, for example, in the blood, blood fraction (e.g., serum), urine, and/or other biological sample from the patient. Any method practiced in the art to detect the compound, or metabolite thereof, may be used to measure the level of the compound during the course of a therapeutic regimen. The dose of a compound described herein may depend upon the patient's condition, that is, stage of the disease, severity of symptoms caused by the disease, general health status, as well as age, gender, and weight, and other factors apparent to a person of ordinary skill in the medical art.

In some embodiments, methods for treating a patient having an advanced and/or metastatic tumor are provided.

In some embodiments of the methods disclosed herein, the advanced and/or metastatic tumor has elevated DGKζ levels.

In some embodiments of the methods disclosed herein, the advanced and/or metastatic tumor is a TIL-poor tumor.

In some embodiments of the methods disclosed herein, the advanced and/or metastatic tumor is an inflamed tumor.

In some embodiments of the methods disclosed herein, the advanced and/or metastatic tumor is chosen from solid tumors. In some embodiments, the solid tumor is chosen from sarcomas, carcinomas, and lymphomas. In some embodiments, the solid tumor is chosen from skin cancer, bladder cancer, breast cancer, uterine cancer, ovary cancer, prostate cancer, lung cancer, colon cancer, pancreas cancer, renal cancer, and gastric cancer.

In some embodiments of the methods disclosed herein, the advanced and/or metastatic tumor is chosen from melanoma and NSCLC. In some embodiments, the advanced and/or metastatic tumor is a melanoma. In some embodiments, the advanced and/or metastatic tumor is a NSCLC.

In some embodiments of the methods disclosed herein, the patient has received, declined or had a contraindication to one or more previous therapies with established clinical benefit for their malignancy. In some embodiments, the patient has received one or more previous therapies with established clinical benefit for their malignancy. In some embodiments, the patient has previously declined one or more previous therapies with established clinical benefit for their malignancy. In some embodiments, the patient has previously had a contraindication to one or more previous therapies with established clinical benefit for their malignancy.

In some embodiments of the methods disclosed herein, the patient was previously treated with an anti-PD-1 therapy. In some embodiments, the patient was previously treated with pembrolizumab, nivolumab, and/or cemiplimab.

In some embodiments of the methods disclosed herein, the patient does not have human immunodeficiency virus (HIV) and/or a compromised immune system. In some embodiments, the patient does not have HIV. In some embodiments, the patient does not have a compromised immune system.

In some embodiments of the methods disclosed herein, the patient is female as assigned at birth. In some embodiments of the methods disclosed herein, the patient is male as assigned at birth.

In some embodiments of the methods disclosed herein, the patient satisfies at least one of the following conditions:
(a) the patient is considered an adult according to local regulation at the time of treatment;
(b) the patient has locally advanced (unresectable) or metastatic solid tumor malignancy, wherein the malignancy is confirmed by available pathology records or current biopsy;
(c) the patient has at least 1 measurable lesion per Response Evaluation Criteria in Solid Tumors (RECIST) v1.1, wherein lesions situated in a previously irradiated area are considered measurable if progression has been demonstrated in such lesions;
(d) the patient has progressed after receiving all standard approved therapies and/or is no longer eligible for standard therapy;
(e) the patient has an Eastern Cooperative Oncology Group (ECOG) Performance Status of 0, 1, or 2;
(f) the patient's last dose of any prior antineoplastic therapy, including any immunotherapy, was at least 21 days prior to being treated by the method disclosed herein; however, for the patient with solid tumors that have a neurotropic receptor tyrosine kinase (NTRK) gene fusion without a known acquired resistance mutation or for the patient with epidermal growth factor receptor (EGFR) or anaplastic lymphomas kinase (ALK) mutation-positive NSCLC, prior NTRK inhibitor or EGFR tyrosine kinase inhibitor (TKI) or ALK inhibitor therapy is allowed until 4 days prior to being treated by the method disclosed herein;
(g) the patient who has received radiotherapy, including stereotactic radiosurgery, must have completed the radiotherapy at least 2 weeks prior to being treated by the method disclosed herein;
(h) the patient's adverse events (excluding alopecia) from a prior therapy has improved to grade 1 or baseline within 14 days prior to being treated by the method disclosed herein; however, the patient that has type 1 diabetes mellitus, an endocrinopathy stably maintained on appropriate replacement therapy, or a skin disorder that does not require systemic treatment are allowed;
(i) the patient has adequate organ function prior to treatment as indicated by the following laboratory values, wherein the laboratory values must be obtained ≥2 weeks after any blood transfusion:
   (i) absolute neutrophil count (ANC) levels ≥1500/μL;
   (ii) Platelets levels ≥100,000/μL;
   (iii) Hemoglobin levels ≥9 g/dL;
   (iv) Creatinine levels either ≤upper limit of normal (ULN) OR creatinine clearance (CLer) ≥60 mL/min as calculated by Cockroft-Gault equation;
   (v) Total bilirubin levels either ≤1.5×ULN; OR, for participants with Gilbert's syndrome, direct bilirubin ≤ULN and total bilirubin <3×ULN;
   (vi) aspartate aminotransferase (AST) (serum glutamic oxaloacetic transaminase (SGOT)) and alanine aminotransferase (ALT) (serum glutamic pyruvic transaminase (SGPT)) levels ≤2.5×ULN without liver metastases or ≤5×ULN if liver metastases are present;
   (vii) Serum potassium levels ≥3.4 mEq/L;
   (viii) Serum magnesium levels ≥1.7 mg/dL; and
   (ix) Serum ionized calcium levels ≥4.7 mg/dL;
(j) the patient has activated partial thromboplastin time (aPTT) and international normalized ratio (INR)≤1.5×ULN and is not receiving anticoagulation;
(k) the patient, if female as assigned at birth, is not pregnant and at least one of the following conditions apply:
   (i) the patient is not a woman of childbearing potential;
   (ii) the patient is a woman of childbearing potential who agrees to follow contraceptive guidance while being treated by the method disclosed herein and for at least 30 days after end of treatment;
(l) the patient, if female as assigned at birth, agrees not to breastfeed during the period starting at screening, while being treated by the method disclosed herein, and for at least 30 days after end of treatment;
(m) the patient, if female as assigned at birth, does not donate ova while being treated by the method disclosed herein and for at least 30 days after end of treatment;
(n) the patient, if male as assigned at birth and has one or more female partners of childbearing potential (including breastfeeding partners), agrees to use contraception while being treated by the method disclosed herein and for at least 30 days after end of treatment;
(o) the patient, if male as assigned at birth, does not donate sperm while being treated by the method disclosed herein and for at least 30 days after end of treatment;
(p) the patient, if male as assigned at birth and has one or more pregnant partners, agrees to remain abstinent or use a condom for the duration of the pregnancy while being treated by the method disclosed herein and for at least 30 days after end of treatment;
(q) the patient agrees not to participate in another interventional study while being treated by the method disclosed herein.

In some embodiments of the methods disclosed herein, the patient satisfies at least two of the conditions (a) through (q) above. In some embodiments of the methods disclosed herein, the patient satisfies at least three of the conditions (a) through (q) above. In some embodiments of the methods disclosed herein, the patient satisfies at least four of the conditions (a) through (q) above. In some embodiments of the methods disclosed herein, the patient satisfies at least five of the conditions (a) through (q) above. In some embodiments of the methods disclosed herein, the patient satisfies at least six of the conditions (a) through (q) above. In some embodiments of the methods disclosed herein, the patient satisfies at least seven of the conditions (a) through (q) above. In some embodiments of the methods disclosed herein, the patient satisfies at least eight of the conditions (a) through (q) above. In some embodiments of the methods disclosed herein, the patient satisfies at least nine of the conditions (a) through (q) above. In some embodiments of the methods disclosed herein, the patient satisfies at least ten of the conditions (a) through (q) above. In some embodiments of the methods disclosed herein, the patient satisfies at least eleven of the conditions (a) through (q) above. In some embodiments of the methods disclosed herein, the patient satisfies at least twelve of the conditions (a) through (q) above. In some embodiments of the methods disclosed herein, the patient satisfies at least thirteen of the conditions (a) through (q) above. In some embodiments of the methods disclosed herein, the patient satisfies at least fourteen of the conditions (a) through (q) above. In some embodiments of the methods disclosed herein, the patient satisfies at least fifteen of the conditions (a) through (q) above. In some embodiments of the methods disclosed herein, the patient satisfies at least sixteen of the conditions (a) through (q) above. In some embodiments of the methods disclosed herein, the patient satisfies each of the conditions (a) through (q) above.

In some embodiments of the methods disclosed herein, the patient has locally advanced (unresectable) or metastatic solid tumor malignancy. In some embodiments, the locally advanced (unresectable) or metastatic solid tumor malignancy is confirmed by available pathology records. In some embodiments, the locally advanced (unresectable) or metastatic solid tumor malignancy is confirmed by current biopsy.

In some embodiments of the methods disclosed herein, the patient has at least 1 measurable lesion per RECIST v1.1. In some embodiments, the patient has at least 1 measurable lesion situated in a previously irradiated area. In some embodiments, progression has been demonstrated in the lesion situated in a previously irradiated area.

In some embodiments of the methods disclosed herein, the patient's last dose of any prior antineoplastic therapy, including any immunotherapy, was at least 21 days prior to being treated by the method disclosed herein. In some embodiments, the patient has solid tumors that have a NTRK gene fusion without a known acquired resistance mutation. In some embodiments of the methods disclosed herein, the patient is allowed to remain on NTRK inhibitors. In some embodiments of the methods disclosed herein, the patient has EGFR or ALK mutation-positive NSCLC until 4 days prior to being treated by the method disclosed herein. In some embodiments of the methods disclosed herein, the patient is allowed to remain on EGFR TKI until 4 days prior to being treated by the method disclosed herein. In some embodiments of the methods disclosed herein, the patient is allowed to remain on ALK inhibitor therapy until 4 days prior to being treated by the method disclosed herein.

In some embodiments of the methods disclosed herein, the patient's adverse events (excluding alopecia) from a prior therapy has improved to grade 1 or baseline within 14 days prior being treated by the method disclosed herein.

In some embodiments of the methods disclosed herein, the patient has type 1 diabetes mellitus, an endocrinopathy stably maintained on appropriate replacement therapy, or a skin disorder that does not require systemic treatment. In some embodiments of the methods disclosed herein, the patient has type 1 diabetes mellitus, an endocrinopathy stably maintained on appropriate replacement therapy, or a skin disorder chosen from vitiligo, psoriasis, and alopecia that does not require systemic treatment are allowed.

In some embodiments of the methods disclosed herein, the patient has adequate organ function prior to treatment.

In some embodiments of the methods disclosed herein, the patient has aPTT and INR≤1.5×ULN.

In some embodiments of the methods disclosed herein, the patient is female as assigned at birth and is not pregnant, and the patient is not a woman of childbearing potential.

In some embodiments of the methods disclosed herein, the patient is female as assigned at birth and is not pregnant, and the patient is a woman of childbearing potential who agrees to follow contraceptive guidance while being treated by the method disclosed herein and for at least 30 days after end of treatment.

In some embodiments of the methods disclosed herein, the patient is excluded from treatment if at least one of the following conditions are satisfied:
(r) the patient has received any investigational therapy (other than possibly an EGFR TKI used by a patient with EGFR-activating mutations, an ALK inhibitor used by a patient with an ALK mutation or an NTRK inhibitor used by a patient with solid tumors that have a NTRK gene fusion without a known acquired resistance mutation) within 21 days or 5 half-lives, whichever is shorter, prior to being treated by the method disclosed herein;
(s) the patient requires or has received systemic steroid therapy or any other immunosuppressive therapy within 14 days prior to being treated by the method disclosed herein; however, patients using a physiologic replacement dose of hydrocortisone or its equivalent, which is defined as up to 30 mg per day of hydrocortisone and up to 10 mg prednisone) are allowed;
(t) the patient requires strong or moderate CYP2D6 inhibitors while being treated by the method described herein;
(u) the patient requires strong CYP3A4 inhibitors while being treated by the method described herein;
(v) the patient has symptomatic central nervous system (CNS) metastases or evidence of unstable CNS metastases even if asymptomatic; however, patients with previously treated CNS metastases are eligible if they are clinically stable and have no evidence of CNS progression by imaging for at least 4 weeks prior to being treated by the method described herein and are not requiring immunosuppressive doses of systemic steroids for no longer than 2 weeks, wherein immunosuppressive doses of systemic steroids comprises >30 mg per day of hydrocortisone or >10 mg per day of prednisone or equivalent;
(w) the patient has an active autoimmune disease; however, patients with type 1 diabetes mellitus, endocrinopathies stably maintained on appropriate replacement therapy, or skin disorders not requiring systemic treatment are allowed;
(x) the patient was discontinued from prior immunomodulatory therapy due to a grade ≥3 toxicity that was mechanistically related to the agent;
(y) the patient is known to have HIV infection; however, patients with HIV has CD4+ T-cell counts ≥350 cells/μL and no history of AIDS-defining opportunistic infections within the past 6 months are eligible;
(z) the patient has any of the following per screening serology test:
   (i) Hepatitis A virus (HAV) antibodies (immunoglobulin M [IgM]);
   (ii) Positive hepatitis B surface antigen (HBsAg) or detectable hepatitis B DNA; however, patients with negative HBsAg, positive hepatitis B core antibody (anti-HIBc) and negative hepatitis B surface antibody (anti-HIBs) are eligible if hepatitis B DNA is undetectable;
   (iii) Hepatitis C virus (HCV) antibodies unless HCV RNA is undetectable
(aa) the patient has received a live vaccine against infectious diseases within 28 days prior being treated by the method described herein;
(bb) the patient has a history of drug-induced pneumonitis (interstitial lung disease) or currently has pneumonitis or a prior history of ILD or noninfectious pneumonitis requiring high-dose glucocorticoids, whether resolved or not;
(cc) the patient has an infection requiring systemic therapy within 14 days prior to being treated by the method described herein;
(dd) the patient has received a prior allogenic bone marrow or solid organ transplant;
(ee) the patient is expected to require another form of antineoplastic therapy while being treated by the method described herein;
(ff) the patient has had a myocardial infarction or unstable angina within 6 months prior to being treated by the method described herein or currently has an uncontrolled illness including, for example, symptomatic congestive heart failure, clinically significant cardiac disease, unstable angina pectoris, cardiac arrhythmia, or psychiatric illness/social situations that would limit compliance with treatment;
(gg) the patient has inadequately controlled hypertension, wherein inadequately controlled hypertension comprises as a systolic blood pressure >150 and/or diastolic blood pressure
   >100 mmHg on antihypertensive medications;
(hh) the patient has a corrected QT interval (Single ECG) using Fridericia's formula (QTcF)
   >450 ms prior to being treated by the method described herein;
(ii) the patient has another malignancy requiring active therapy, except for locally curable malignancies, including for example basal or squamous cell skin cancer, superficial bladder cancer and carcinoma in situ of the cervix or breast;
(jj) the patient has had a major surgical procedure and has not completely recovered within 28 days prior to being treated by the method described herein;
(kk) the patient has a history of bleeding diathesis;
(ll) the patient requires the use of any anticoagulation therapy;
(mm) the patient has been previously treated with a DGK inhibitor;
(nn) the patient has a known or suspected hypersensitivity to the at least one entity, or any components of the pharmaceutical composition used.

In some embodiments of the methods disclosed herein, the patient is excluded from treatment if the patient requires systemic steroid therapy or any other immunosuppressive therapy within 14 days prior to being treated by the method disclosed herein. In some embodiments of the methods disclosed herein, the patient is excluded from treatment if the patient has received systemic steroid therapy or any other immunosuppressive therapy within 14 days prior to being treated by the method disclosed herein.

In some embodiments of the methods disclosed herein, the patient is excluded from treatment if the patient requires one or more strong or moderate CYP2D6 inhibitors while being treated by the method described herein, wherein the one or more strong or moderate CYP2D6 inhibitors are chosen from bupropion, fluoxetine, paroxetine, duloxetine, and abiraterone. In some embodiments of the methods disclosed herein, the patient is excluded from treatment if the patient requires one or more strong CYP2D6 inhibitors while being treated by the method described herein. In some embodiments of the methods disclosed herein, the patient is excluded from treatment if the patient requires one or more moderate CYP2D6 inhibitors while being treated by the method described herein.

In some embodiments of the methods disclosed herein, the patient is excluded from treatment if the patient requires one or more strong CYP3A4 inhibitors while being treated by the method described herein, wherein the one or more strong CYP3A4 inhibitors are chosen from boceprevir, clarithromycin, cobicistat, indinavir, itraconazole, nefazodone, nelfinavir, posaconazole, ritonavir, saquinavir, telaprevir, telithromycin, and voriconazole.

In some embodiments of the methods disclosed herein, the patient is excluded from treatment if the patient has symptomatic CNS metastases.

In some embodiments of the methods disclosed herein, the patient is excluded from treatment if the patient has evidence of unstable CNS metastases even if asymptomatic.

In some embodiments of the methods disclosed herein, the patient is excluded from treatment if the patient was discontinued from prior immunomodulatory therapy due to a grade ≥3 toxicity that was mechanistically related (e.g., immune-related) to the agent.

In some embodiments of the methods disclosed herein, the patient is excluded from treatment if the patient is known to have HIV infection.

In some embodiments of the methods disclosed herein, the patient is excluded from treatment if the patient has HAV antibodies (IgM) per screening serology test. In some embodiments of the methods disclosed herein, the patient is excluded from treatment if the patient has Positive HBsAg per screening serology test. In some embodiments, the patient is excluded from treatment if the patient has detectable hepatitis B DNA per screening serology test. In some embodiments of the methods disclosed herein, the patient is excluded from treatment if the patient has HCV antibodies unless HCV RNA is undetectable per screening serology test.

In some embodiments of the methods disclosed herein, the patient is excluded from treatment if the patient has a history of drug-induced pneumonitis (interstitial lung disease), whether resolved or not. In some embodiments of the methods disclosed herein, the patient is excluded from treatment if the patient currently has pneumonitis. In some embodiments of the methods disclosed herein, the patient is excluded from treatment if the patient has ILD, whether resolved or not.

In some embodiments of the methods disclosed herein, the patient is excluded from treatment if the patient has a prior history of noninfectious pneumonitis requiring high-dose glucocorticoids, whether resolved or not.

In some embodiments of the methods disclosed herein, the patient is excluded from treatment if the patient has received a prior allogenic bone marrow transplant. In some embodiments of the methods disclosed herein, the patient is excluded from treatment if the patient has received a prior solid organ transplant.

In some embodiments of the methods disclosed herein, the patient is excluded from treatment if the patient has had a myocardial infarction within 6 months prior to being treated by the method described herein. In some embodiments of the methods disclosed herein, the patient is excluded from treatment if the patient has had an unstable angina within 6 months prior to being treated by the method described herein. In some embodiments of the methods disclosed herein, the patient is excluded from treatment if the patient currently has an uncontrolled illness. In some embodiments, the uncontrolled illness is chosen from symptomatic congestive heart failure, clinically significant cardiac disease, unstable angina pectoris, cardiac arrhythmia, and psychiatric illness/social situations that would limit compliance with treatment.

In some embodiments of the methods disclosed herein, the patient is excluded from treatment if the patient has inadequately controlled hypertension. In some embodiments of the methods disclosed herein, the patient is excluded from treatment if the patient has a systolic blood pressure >150 and/or diastolic blood pressure >100 mmHg on antihypertensive medications. In some embodiments of the methods disclosed herein, the patient is excluded from treatment if the patient has a systolic blood pressure >150 on antihypertensive medications. In some embodiments of the methods disclosed herein, the patient is excluded from treatment if the patient has a diastolic blood pressure >100 mmHg on antihypertensive medications.

In some embodiments of the methods disclosed herein, the patient is excluded from treatment if the patient has another malignancy requiring active therapy. In some embodiments of the methods disclosed herein, the patient is excluded from treatment if the patient has another malignancy requiring active therapy, except for locally curable malignancies. In some embodiments, the locally curable malignancies are chosen from basal or squamous cell skin cancer, superficial bladder cancer and carcinoma in situ of the cervix or breast.

In some embodiments of the methods disclosed herein, the patient is excluded from treatment if the patient has a known hypersensitivity to the at least one entity, or any components of the pharmaceutical composition used. In some embodiments of the methods disclosed herein, the patient is excluded from treatment if the patient has a suspected hypersensitivity to the at least one entity, or any components of the pharmaceutical composition used.

In some embodiments of the methods disclosed herein, treatment with the at least one entity or pharmaceutical composition comprising same may result in activation of immune cells. In some embodiments of the methods disclosed herein, treatment with the at least one entity or pharmaceutical composition comprising same may result in activation of T-cells. In some embodiments of the methods disclosed herein, treatment with the at least one entity or pharmaceutical composition comprising same suppresses the growth of at least one tumor.

In some embodiments of the methods disclosed herein, treatment with the at least one entity or pharmaceutical composition comprising same suppresses the growth of at least one tumor by activation of T-cells. In some embodiments of the methods disclosed herein, treatment with the at least one entity or pharmaceutical composition comprising same suppresses the growth of at least one tumor by inhibiting DGK.

In some embodiments of the methods disclosed herein, treatment with the at least one entity or pharmaceutical compositions comprising same slows down the progression of the cancer. In some embodiments of the methods disclosed herein, treatment with the at least one entity or pharmaceutical composition comprising same lessens the symptoms of the cancer. In some embodiments of the methods disclosed herein, treatment with the at least one entity or pharmaceutical composition comprising same halts progression of the cancer. In some embodiments of the methods disclosed herein, treatment with the at least one entity or pharmaceutical composition comprising cures the cancer.

In some embodiments of the methods disclosed herein, the cancer is melanoma. In some embodiments, the cancer is NSCLC. In some embodiments, the cancer is small cell lung cancer. In some embodiments, the cancer is head and neck cancer. In some embodiments, the cancer is kidney cancer. In some embodiments, the cancer is ovarian cancer. In some embodiments, the cancer is mismatch repair-deficient colon cancer. In some embodiments, the cancer is urothelial cancer. In some embodiments, the cancer is hepatocellular carcinoma. In some embodiments, the cancer is gastric cancer. In some embodiments, the cancer is bladder cancer.

In some embodiments, administration is oral administration. In some embodiments, the pharmaceutical composition is suitable for oral administration.

In some embodiments, the pharmaceutical composition of the present disclosure can be prepared by a commonly used method with one or more excipients commonly used in the art, i.e., an excipient for pharmaceutical use, a carrier for pharmaceutical use, or the like.

In some embodiments, the pharmaceutical composition is in the form of a tablet, pill, capsule, granule, powder, solution, or the like. In some embodiments, the pharmaceutical composition is in the form of a tablet. In some embodiments, the pharmaceutical composition is in the form of a pill. In some embodiments, the pharmaceutical composition is in the form of a capsule. In some embodiments, the pharmaceutical composition is in the form of a granule. In some embodiments, the pharmaceutical composition is in the form of a powder. In some embodiments, the pharmaceutical composition is in the form of a solution.

In some embodiments, the pharmaceutical composition may contain inactive additives, for example a lubricant, a disintegrant, a stabilizer and a solubilizing agent. The tablet, powder, granule or pill may be coated with a wax, a sugarcoating or a stomach-soluble or enteric substance film. Liquid compositions for oral administration may include pharmaceutically acceptable emulsions, solutions, suspensions, syrups or elixirs, and may contain inactive diluents, for example purified water or ethanol. Such a liquid composition may contain adjuvants such as a solubilizer, a wetting agent and a suspension, a sweetening agent, a flavor, a fragrance and a preservative in addition to the inactive diluent.

In some embodiments of the methods disclosed herein, the at least one entity is administered orally at a dose equivalent of about 5 mg/day, about 10 mg/day, about 30 mg/day, about 60 mg/day, about 100 mg/day, about 150 mg/day, or about 200 mg/day Compound I. In some embodiments, the at least one entity is administered orally at a dose equivalent of about 10 mg/day Compound I. In some embodiments, the at least one entity is administered orally at a dose equivalent of about 30 mg/day Compound I. In some embodiments, the at least one entity is administered orally at a dose equivalent of about 60 mg/day Compound I. In some embodiments, the at least one entity is administered orally at a dose equivalent of about 100 mg/day Compound I. In some embodiments, the at least one entity is administered orally at a dose equivalent of about 150 mg/day Compound I. In some embodiments, the at least one entity is administered orally at a dose equivalent of about 200 mg/day Compound I.

In some embodiments of the methods disclosed herein, the at least one entity is administered in increasing dose equivalent amounts Compound I in a stepwise manner. In some embodiments, the at least one entity is administered at an initial dose equivalent of about 5 mg/day Compound I for a first period of time and wherein a second dose equivalent amount Compound I of approximately 2.0 to 4.0 times greater than the first dose equivalent amount is administered for a second period of time. In some embodiments, the at least one entity is administered at an initial dose equivalent of about 10 mg/day Compound I for a first period of time and wherein a second dose equivalent amount Compound I of approximately 1.5 to 3.0 times greater than the first dose equivalent amount is administered for a second period of time. In some embodiments, the at least one entity is administered at an initial dose equivalent of about 30 mg/day Compound I for a first period of time and wherein a second dose equivalent amount Compound I of approximately 1.5 to 3.0 times greater than the first dose equivalent amount is administered for a second period of time. In some embodiments, the at least one entity is administered at an initial dose equivalent of about 60 mg/day Compound I for a first period of time and wherein a second dose equivalent amount Compound I of approximately 1.5 to 3.0 times greater than the first dose equivalent amount is administered for a second period of time. In some embodiments, the at least one entity is administered at an initial dose equivalent of about 100 mg/day Compound I for a first period of time and wherein a second dose equivalent amount Compound I of approximately 1.5 to 2.0 times greater than the first dose equivalent amount is administered for a second period of time. In some embodiments, the at least one entity is administered at an initial dose equivalent of about 150 mg/day Compound I for a first period of time and wherein a second dose equivalent amount Compound I of approximately 1.2 to 1.5 times greater than the first dose equivalent amount is administered for a second period of time.

In some embodiments of the methods disclosed herein, the method further comprises administering a third dose equivalent amount Compound I of approximately 1.5 to 2.5 times greater than the second dose equivalent amount for a third period.

In some embodiments of the methods disclosed herein, the time periods for each administration are up to 21 days.

In some embodiments, the dosage escalation regime provides a positive patient safety profile and suitable peak plasma concentration of drug.

In some embodiments, the at least one entity is administered orally at a daily dose equivalent of about 5 mg/day to about 200 mg/day Compound I. In some embodiments, the at least one entity is administered orally at a daily dose equivalent of about 10 mg/day to about 200 mg/day Compound I. In some embodiments, the at least one entity is administered orally at a daily dose equivalent of about 30 mg/day to about 200 mg/day Compound I. In some embodiments, the at least one entity is administered orally at a daily dose equivalent of about 60 mg/day to about 200 mg/day Compound I. In some embodiments, the at least one entity is administered orally at a daily dose equivalent of about 100 mg/day to about 200 mg/day Compound I. In some embodiments, the at least one entity is administered orally at a daily dose equivalent of about 5 mg/day to about 100 mg/day Compound I. In some embodiments, the at least one entity is administered orally at a daily dose equivalent of about 10 mg/day to about 100 mg/day Compound I. In some embodiments, the at least one entity is administered orally at a daily dose equivalent of about 30 mg/day to about 100 mg/day Compound I. In some embodiments, the at least one entity is administered orally at a daily dose equivalent of about 60 mg/day to about 100 mg/day Compound I. In some embodiments, the at least one entity is administered orally at a daily dose equivalent of about 30 mg/day to about 200 mg/day Compound I. In some embodiments, the at least one entity is administered orally at a daily dose equivalent of about 30 mg/day to about 150 mg/day Compound I. In some embodiments, the at least one entity is administered orally at a daily dose equivalent of about 30 mg/day to about 100 mg/day Compound I.

In some embodiments, the daily dose equivalent is administered orally in a single dose. In some embodiments, the daily dose is administered orally in 2 to 4 divided doses.

In some embodiments, the pharmaceutical composition for oral administration comprises a dose equivalent of about 5 mg, about 10 mg, about 30 mg, about 60 mg, about 100 mg, about 150 mg, or about 200 mg Compound I. In some embodiments, the pharmaceutical composition for oral administration comprises a dose equivalent of about 10 mg Compound I. In some embodiments, the pharmaceutical composition for oral administration comprises a dose equivalent of about 30 mg Compound I. In some embodiments, the pharmaceutical composition for oral administration comprises a dose equivalent of about 60 mg Compound I. In some embodiments, the pharmaceutical composition for oral administration comprises a dose equivalent of about 100 mg Compound I. In some embodiments, the pharmaceutical composition for oral administration comprises a dose equivalent of about 150 mg Compound I. In some embodiments, the pharmaceutical composition for oral administration comprises a dose equivalent of about 200 mg Compound I.

In some embodiments, the pharmaceutical composition for oral administration comprises a dose equivalent of about 5 mg to about 200 mg Compound I. In some embodiments, the pharmaceutical composition for oral administration comprises a dose equivalent of about 10 mg to about 200 mg Compound I. In some embodiments, the pharmaceutical composition for oral administration comprises a dose equivalent of about 30 mg to about 200 mg Compound I. In some embodiments, the pharmaceutical composition for oral administration comprises a dose equivalent of about 60 mg to about 200 mg Compound I. In some embodiments, the pharmaceutical composition for oral administration comprises a dose equivalent of about 100 mg to about 200 mg Compound I. In some embodiments, the pharmaceutical composition for oral administration comprises a dose equivalent of about 5 mg to about 100 mg Compound I. In some embodiments, the pharmaceutical composition for oral administration comprises a dose equivalent of about 10 mg to about 100 mg Compound I. In some embodiments, the pharmaceutical composition for oral administration comprises a dose equivalent of about 30 mg to about 100 mg Compound I. In some embodiments, the pharmaceutical composition for oral administration comprises a dose equivalent of about 60 mg to about 100 mg Compound I. In some embodiments, the pharmaceutical composition for oral administration comprises a dose equivalent of about 30 mg to about 200 mg Compound I. In some embodiments, the pharmaceutical composition for oral administration comprises a dose equivalent of about 30 mg to about 150 mg Compound I. In some embodiments, the pharmaceutical composition for oral administration comprises a dose equivalent of about 30 mg to about 100 mg/Compound I.

As will be apparent to those skilled in the art, dosages outside of these disclosed dosages and ranges may be administered in some cases. Further, it is noted that the ordinary skilled clinician or treating physician will know how and when to interrupt, adjust, or terminate therapy in consideration of individual response.

One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### EXAMPLES

### Example 1

### Synthesis of Compound I

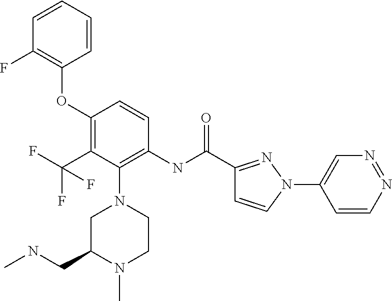 [SMILES: CNC[C@H]1CN(c2c(NC(=O)c3ccn(-c4ccnnc4)n3)ccc(Oc3ccccc3F)c2C(F)(F)F)CCN1C]

2-Bromo-4-(2-fluorophenoxy)-1-nitro-3-(trifluoromethyl)benzene: A mixture of 2-bromo-4-fluoro-1-nitro-3-(trifluoromethyl)benzene (3.00 g), 2-fluorophenol (1.00 mL), potassium carbonate (2.88 g) and NMP (30 mL) was stirred at 50° C. overnight. The mixture was allowed to cool to room temperature, water, EtOAc and brine were then added, and the aqueous layer was separated. The aqueous layer was extracted with EtOAc, and the combined organic layers were dried over MgSO₄, and concentrated under reduced pressure. The residue was purified by silica gel chromatography (Hex/EtOAc) to give 2-bromo-4-(2-fluorophenoxy)-1-nitro-3-(trifluoromethyl)benzene (3.43 g).

Tert-Butyl {[(2R)-4-benzyl-1-methylpiperazin-2-yl]methyl}(methyl)carbamate: A solution of [(2R)-4-benzyl-1-methylpiperazin-2-yl]methanol (5.27 g), TEA (6.7 mL) and THF (100 mL) was cooled in an ice-MeOH bath, and methanesulfonyl chloride (1.96 mL) was then slowly added. The resulting mixture was stirred under ice-bath cooling for 1 hour, a 40% methylamine aqueous solution (40 mL) was added, and the resulting mixture was then stirred at 70° C. for 3 hours, and allowed to cool. The reaction liquid was then concentrated under reduced pressure, water and CH₂Cl₂ were added to the residue, and the aqueous layer was separated. The aqueous layer was extracted with CH₂Cl₂ twice, and the combined organic layers were dried over Na₂SO₄, and then concentrated under reduced pressure.

The resulting oily substance (4.95 g) was dissolved in CH₂Cl₂ (100 mL), and di-tert-butyl dicarbonate (11 g) was added under ice-bath cooling. The resulting mixture was stirred at room temperature for 2 hours. CH₂Cl₂ and water were added to the reaction mixture, and the aqueous layer was separated. The aqueous layer was extracted with CH₂Cl₂, and the combined organic layers were dried over Na₂SO₄, and then concentrated under reduced pressure. The residue was purified by silica gel column chromatography (aqueous ammonia/MeOH/chloroform) to give tert-butyl {[(2R)-4-benzyl-1-methylpiperazin-2-yl]methyl}(methyl)carbamate (5.92 g).

Tert-Butyl methyl{[(2R)-1-methylpiperazin-2-yl]methyl}carbamate: To a solution of tert-butyl {[(2R)-4-benzyl-1-methylpiperazin-2-yl]methyl}(methyl)carbamate (4.92 g) and EtOH (100 mL) was added activated carbon (500 mg), and the resulting mixture was stirred at room temperature for 10 minutes. The mixture was filtered with celite, and the filtrate was then concentrated under reduced pressure. To the resulting solution of an oily substance and EtOH (100 mL) was added 10% Pd/C (hydrous, 510 mg) under a nitrogen atmosphere. The reaction mixture was stirred under a hydrogen atmosphere at room temperature for 24 hours. The reaction mixture was filtered with celite, and the filtrate was then concentrated under reduced pressure to give tert-butyl methyl{[(2R)-1-methylpiperazin-2-yl]methyl}carbamate (3.43 g).

Tert-Butyl ({(2R)-4-[3-(2-fluorophenoxy)-6-nitro-2-(trifluoromethyl)phenyl]-1-methylpiperazin-2-yl}methyl)(methyl)carbamate: A mixture of tert-butyl methyl{[(2R)-1-methylpiperazin-2-yl]methyl}carbamate (735 mg), 2-bromo-4-(2-fluorophenoxy)-1-nitro-3-(trifluoromethyl)benzene (1.15 g), potassium carbonate (627 mg) and 1,4-dioxane (5.75 mL) was stirred at 110° C. for 24 hours, and allowed to cool. The reaction mixture was then diluted with EtOAc. The mixture was filtered with celite, and the filtrate was then concentrated under reduced pressure. The residue was purified by silica gel column chromatography (Hex/EtOAc) to give tert-butyl ({(2R)-4-[3-(2-fluorophenoxy)-6-nitro-2-(trifluoromethyl)phenyl]-1-methylpiperazin-2-yl}methyl)(methyl)carbamate (1.31 g).

Tert-Butyl ({(2R)-4-[6-amino-3-(2-fluorophenoxy)-2-(trifluoromethyl)phenyl]-1-methylpiperazin-2-yl}methyl)(methyl)carbamate: To a solution of tert-butyl ({(2R)-4-[3-(2-fluorophenoxy)-6-nitro-2-(trifluoromethyl)phenyl]-1-methylpiperazin-2-yl}methyl)(methyl)carbamate (3.41 g) in 1,4-dioxane (50 mL) was added a solution of NH₄Cl (3.36 g) in water (25 mL), zinc powder (4.11 g) was then added under ice-bath cooling, and the resulting mixture was stirred at room temperature for 3 hours. The reaction mixture was diluted with EtOAc and water, and then filtered with celite. The aqueous layer of the filtrate was separated. The aqueous layer was extracted with EtOAc, and the combined organic layers were dried over Na₂SO₄, and concentrated under reduced pressure to give tert-butyl ({(2R)-4-[6-amino-3-(2-fluorophenoxy)-2-(trifluoromethyl)phenyl]-1-methylpiperazin-2-yl}methyl)(methyl)carbamate (3.32 g).

Tert-Butyl ({(2R)-4-[3-(2-fluorophenoxy)-6-{[1-(pyridazin-4-yl)-1H-pyrazole-3-carbonyl]amino}-2-(trifluoromethyl)phenyl]-1-methylpiperazin-2-yl}methyl)(methyl)carbamate: To a mixture of tert-butyl ({(2R)-4-[6-amino-3-(2-fluorophenoxy)-2-(trifluoromethyl)phenyl]-1-methylpiperazin-2-yl}methyl)(methyl)carbamate (3.22 g), 1-(pyridazin-4-yl)-1H-pyrazole-3-carboxylic acid (1.43 g), DIPEA (3.3 mL) and DMF (50 mL) was added HATU (3.58 g). The reaction mixture was stirred at 50° C. for 12 hours and allowed to cool to room temperature. Chloroform and water were added to the reaction mixture, and the aqueous layer was separated. The aqueous layer was extracted with chloroform, and the combined organic layers were dried over Na₂SO₄, and concentrated under reduced pressure. The residue was purified by silica gel column chromatography (chloroform/MeOH) to give tert-butyl ({(2R)-4-[3-(2-fluorophenoxy)-6-{[1-(pyridazin-4-yl)-1H-pyrazole-3-carbonyl]amino}-2-(trifluoromethyl)phenyl]-1-methylpiperazin-2-yl}methyl)(methyl)carbamate (4.35 g).

N-[4-(2-Fluorophenoxy)-2-{(3S)-4-methyl-3-[(methylamino)methyl]piperazin-1-yl}-3-(trifluoromethyl)phenyl]-1-(pyridazin-4-yl)-1H-pyrazole-3-carboxamide: To a mixture of tert-butyl ({(2R)-4-[3-(2-fluorophenoxy)-6-{[1-(pyridazin-4-yl)-1H-pyrazole-3-carbonyl]amino}-2-(trifluoromethyl)phenyl]-1-methylpiperazin-2-yl}methyl)(methyl)carbamate (4.3 g) and CH₂Cl₂ (40 mL) was added TFA (10 mL), and the resulting mixture was stirred at room temperature for 4 hours. The reaction liquid was concentrated under reduced pressure, and the residue was basified by addition of water, chloroform and NaHCO₃ thereto. A liquid separation process was carried out with a separatory funnel, the aqueous layer was extracted with chloroform, and the combined organic layers were dried over Na₂SO₄, and concentrated under reduced pressure. The residue was purified by silica gel column chromatography (chloroform/MeOH/aqueous ammonia). The resulting amorphous substance was washed with EtOAc/Hex to give N-[4-(2-fluorophenoxy)-2-{(3 S)-4-methyl-3-[(methylamino)methyl]piperazin-1-yl}-3-(trifluoromethyl)phenyl]-1-(pyridazin-4-yl)-1H-pyrazole-3-carboxamide (2.62 g) as a solid substance.

### Example 2

A Phase 1/2 Study of Compound I in Participants with Locally Advanced or Metastatic Solid Tumors

This is a planned phase 1/2, open-label, multi-center, multiple-dose, dose escalation and expansion study of Compound I, a selective small molecule inhibitor of DGK, in participants with advanced/metastatic solid tumors.

### Objectives and Endpoints

The study objectives and endpoints are shown in Table 1, below.

TABLE 1
Study Objectives and Endpoints

| | |
|---|---|
| Objectives | Endpoints |

Primary

| | |
|---|---|
| To determine the safety and | Safety variables (e.g., incidence of DLTs |
| tolerability of Compound I | and AEs; change from baseline in |
| To determine the RP2D and/or MTD | laboratory tests, vital signs and ECG) |
| of Compound I | |

Secondary

| | |
|---|---|
| To evaluate the anti-tumor effects of | ORR, DOR, DCR, PFS per iRECIST and |
| Compound I | RECIST 1.1 and OS of Compound I |
| To evaluate pharmacokinetics of | Selected pharmacokinetic parameters of |
| Compound I | Compound I in plasma: Cₘₐₓ, tₘₐₓ, AUC₂₄ |
| To evaluate the effect of Compound I | and C_{trough} |
| on level and proliferative index of | Changes in tumor infiltration with |
| TILs in tumor microenvironment | CD4/CD8 cells and level of their |
| | proliferation (CD4/CD8, Ki67+) |

Exploratory

| | |
|---|---|
| To evaluate exploratory | Changes in exploratory |
| pharmacodynamic biomarkers that | pharmacodynamics of tumor and |
| may correlate with Compound I | peripheral blood biomarkers confirming |
| activity | activation of systemic anti-tumor immune |
| To evaluate exploratory predictive | response |
| biomarkers that monitor the status | Exploratory peripheral and tumor |
| of the immune system or may | biomarker levels that may correlate with |
| correlate with treatment outcome | treatment outcome |
| To evaluate additional | AUC_{inf} (% extrap), CL/F, t_{lag}, t_{1/2} and V_{z}/F |
| pharmacokinetic parameters of | |
| Compound I | |

AUC: area under the curve;
AE: adverse event;
DCR: disease control rate;
DLT: dose-limiting toxicity;
DOR: duration of response;
ECG: electrocardiogram;
iRECIST: Immune Response Evaluation Criteria in Solid Tumors;
MTD: maximum tolerated dose;
ORR: objective response rate;
RECIST: Response Evaluation Criteria in Solid Tumors;
RP2D: recommended phase 2 dose

### Study Design and Dose Rationale

### Study Design and Dose Rationale: Overall Study Design

The study consists of 2 parts: Dose Escalation Phase and Dose Expansion Phase. For all participants, the study will consist of the following periods: Screening (up to 28 days); Treatment (daily dosing in 21-day cycles until treatment discontinuation criteria are met); End of Treatment (after discontinuation of study drug treatment); and Follow-up (safety follow-up visits and survival follow-up).

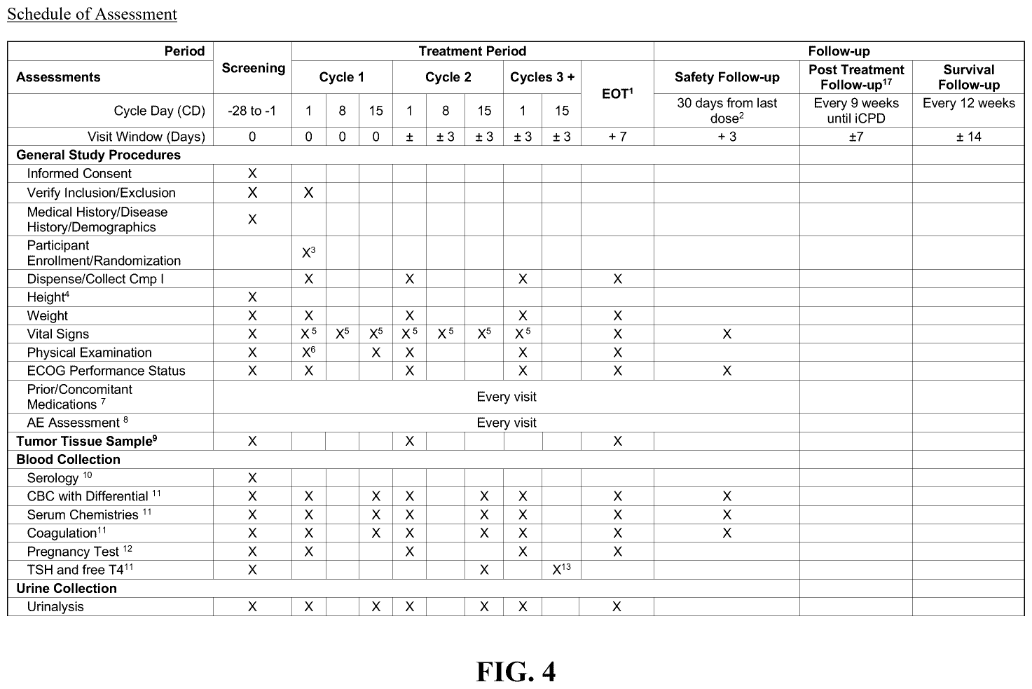

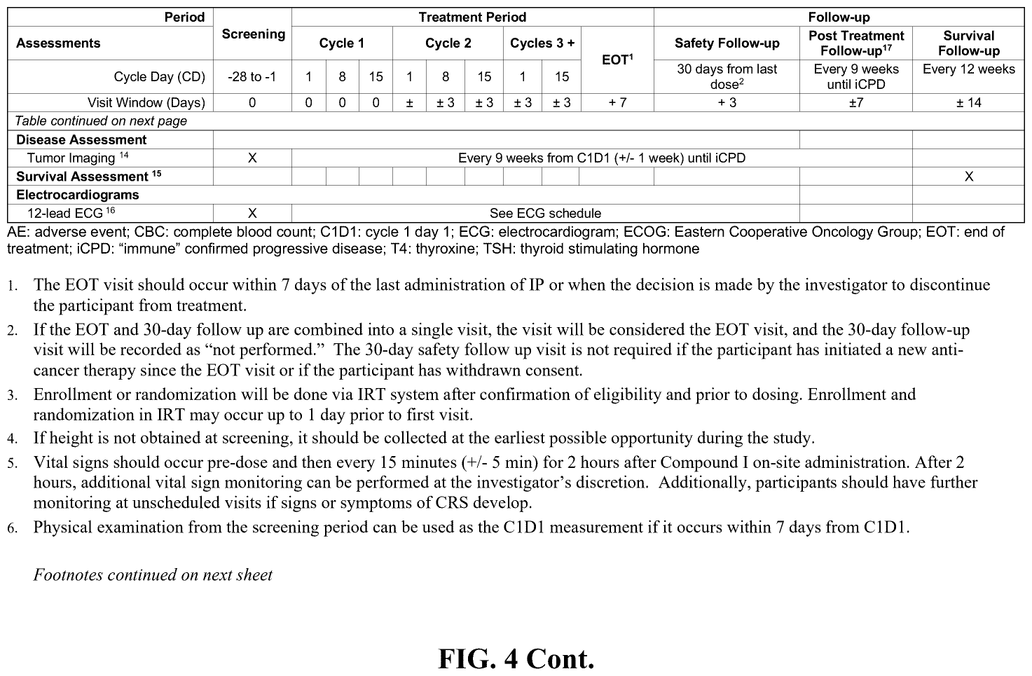

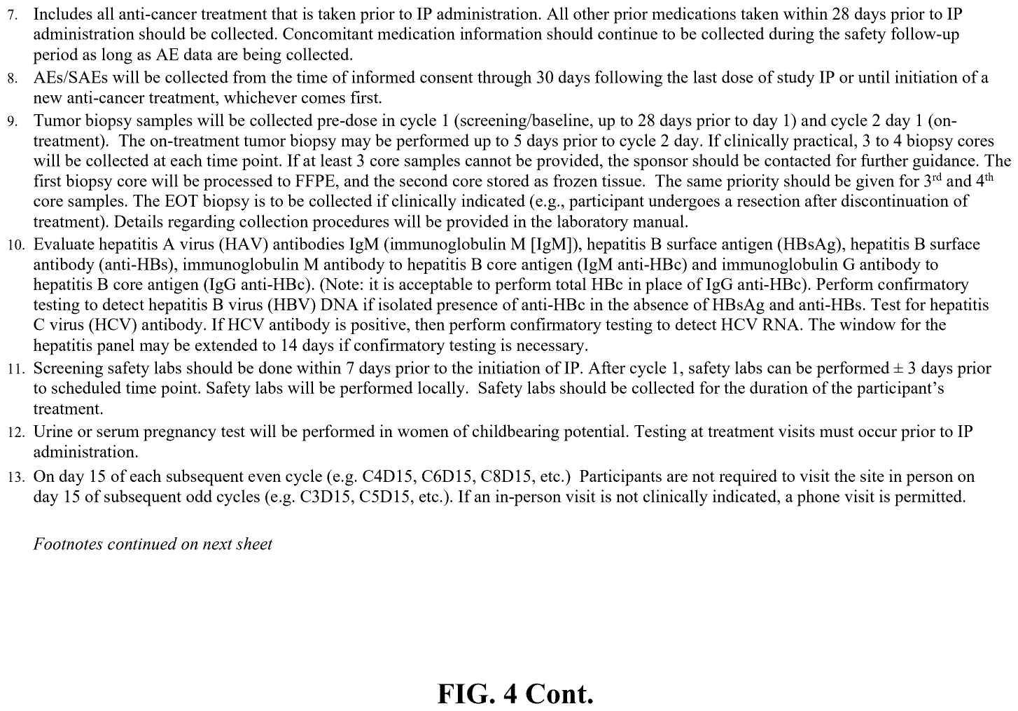

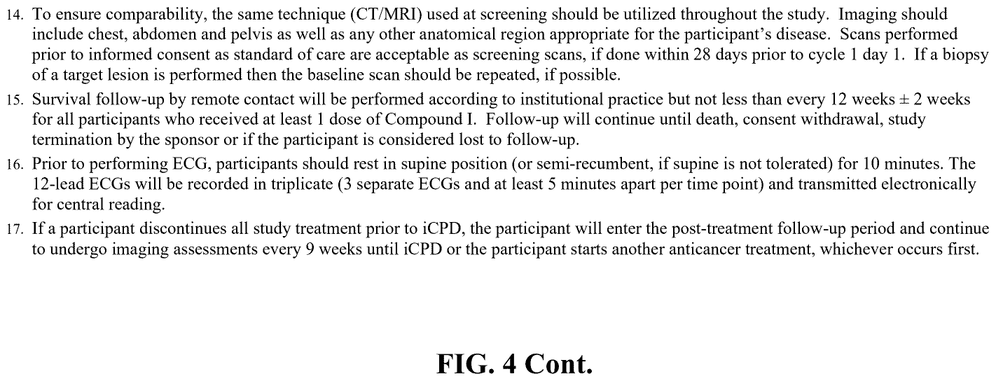

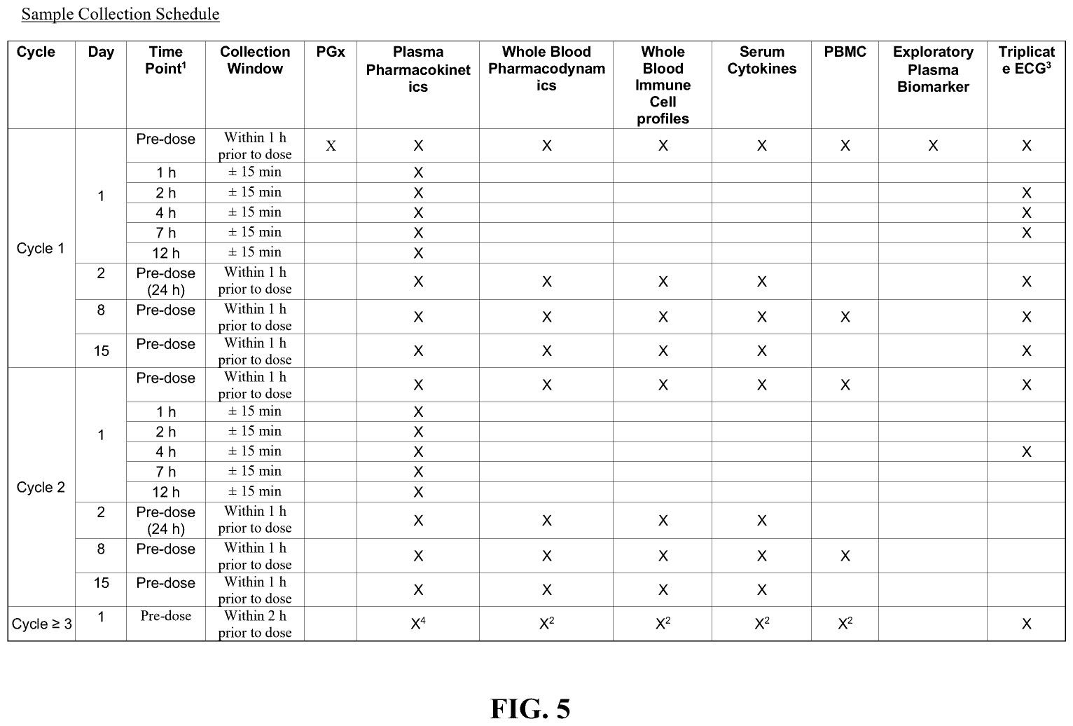

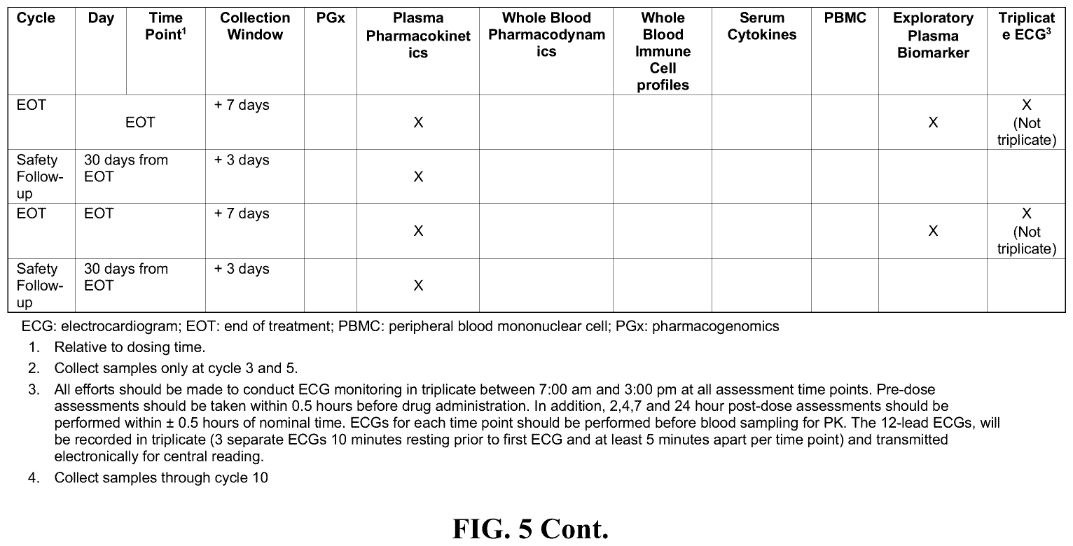

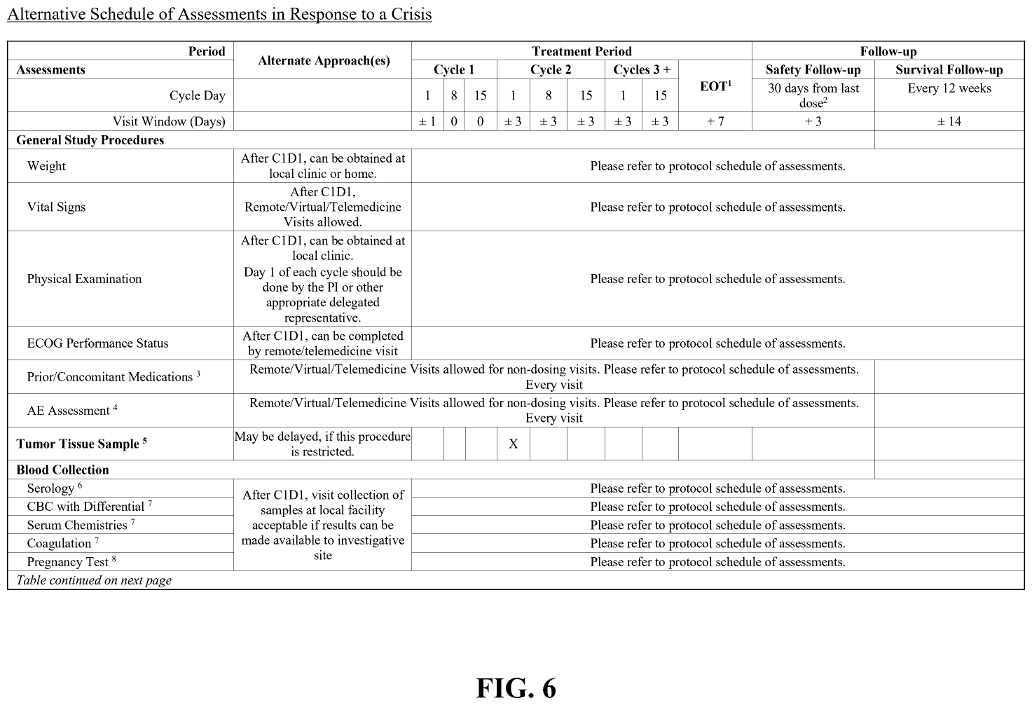

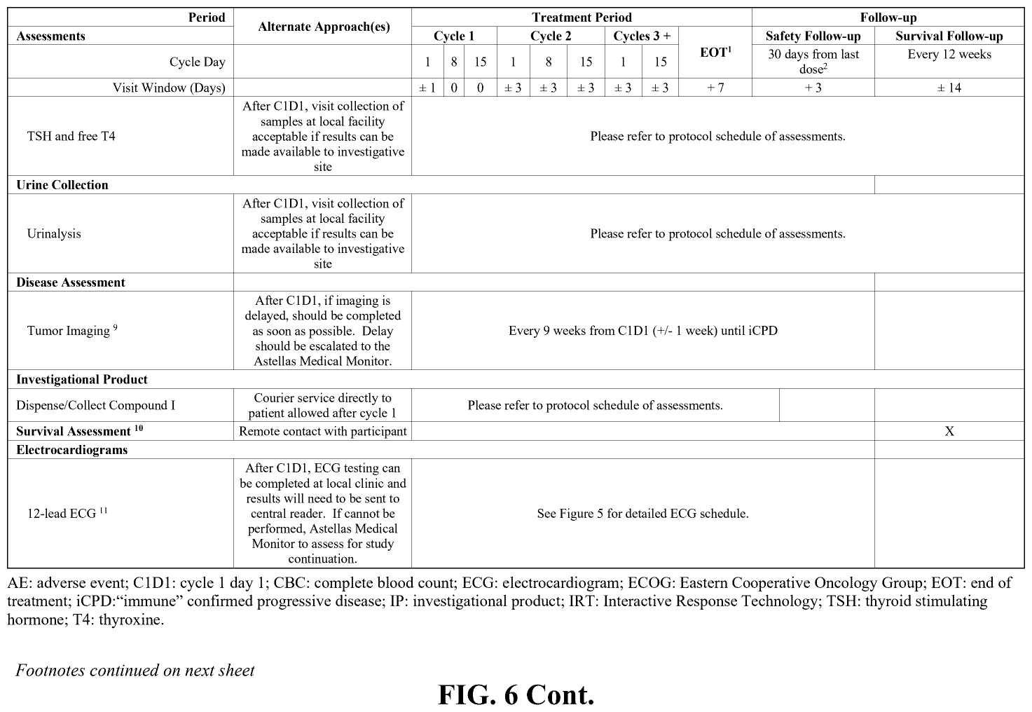

A Schedule of Assessment and a Sample Collection Schedule are shown in FIGS. 4 and 5, respectively.

After discontinuation of study drug treatment, all participants will complete an end of treatment visit up to 7 days following the decision to discontinue. Participants will then enter the safety follow-up period and have a safety follow-up visit at 30 days from date of last dose. The only exception to this is when the participant specifically withdraws consent for any further contact with him/her or persons previously authorized by the participant to provide this information. If the participant has started a new anti-cancer therapy, he/she will be followed for survival.

Participants will then be followed for survival according to institutional guidelines, but not less than every 12 weeks after the final safety follow-up visit as outlined in the Schedule of Assessments (FIG. 4) and until death, withdrawal of consent or study closure, whichever occurs first.

### Part 1: Dose Escalation

The dose escalation cohorts will evaluate escalating dose levels of Compound I in up to approximately 36 DLT-evaluable participants. A cycle is defined as 21 days (3 weeks). Dosing occurs daily on each day of the cycle. Participants will be evaluated for DLTs during the first 21 days (cycle 1). Safety and tolerability will be continually assessed from day 1 through 30 days after the last dose of Compound I.

There will be at least 3 calendar days between the treatment initiation of the first participant and all subsequent participants in the same dose level for all escalation cohorts.

Dose escalation or de-escalation will be guided according to the time-to-event Bayesian Optimal Interval (TITE-BOIN) design (Yuan et al, 2018) to determine the next dose level based on DLT occurrence.

For each dose level, after the planned number of evaluable participants have completed the DLT observation period, safety for that dose level will be assessed. The below listed proposed dose escalation levels, Table 2, are based on minimum pharmacologic activity and nonclinical toxicology study results.

TABLE 2
Proposed Dose Escalation Cohorts of Compound I

| | Compound I | | Planned Number |
|---|---|---|---|
| Dose Level ^{a} | Daily Dose ^{b} | | of Participants |
| 1 | 10 | mg po qd | At least 1 |
| 2 | 30 | mg po qd | At least 3 |
| 3 | 60 | mg po qd | At least 3 |
| 4 | 100 | mg po qd | At least 3 |
| 5 | 150 | mg po qd | At least 3 |
| 6 | 200 | mg po qd | At least 3 |

^{a} Dose level −1 (5 mg qd) will only be studied if de-escalation is needed after enrollment of dose level 1.
^{b} Higher top dose or intermediate doses may be explored based on available clinical data.

### TITE-BOIN Design for Escalation Cohorts

The target toxicity rate for the Maximum Tolerated Dose (MTD) is 0.30. The length of the DLT observation period is 21 days. Dose level 1 will enroll an evaluable participant for the initial assessment of DLT. If the participant has no DLTs, escalate the dose to the next higher dose level. If the participant has a DLT, two additional evaluable participants may be added at dose level 1. Hereafter, each dose level will enroll at least 3 evaluable participants for the initial assessment of DLT. If additional participants are needed for a dose level, an additional at least 3 evaluable participants may be added. The dose escalation/de-escalation process can be stopped if the number of participants treated at the current dose reaches at least 6, and the decision according to TITE-BOIN design is to stay at the current dose. The maximum number of participants in a dose escalation cohort is 12 at each dose level.

The TITE-BOIN design uses the following rule, optimized to minimize the probability of incorrect dose assignment, to guide dose escalation/de-escalation:
If the predicted DLT rate at the current dose is ≤0.236, escalate the dose to the next higher dose level;
If the predicted DLT rate at the current dose is >0.359, de-escalate the dose to the next lower dose level;
Otherwise, stay at the current dose.

To assign a dose to the next cohort of participants, count the number of evaluable participants (No. Evaluable), the number of participants who experienced DLT (No. DLTs), and the number of pending participants (No. Pending) and their weighted standardized total follow-up time (STFT) at the current dose, and then make the dose escalation/de-escalation decision according to the rule corresponding to the cumulative number of participants 3, 6, or 9 (see Table 3). If the cumulative number of participants is not 3, 6, or 9, the next dose will be determined according to the predicted DLT rate and the optimal interval boundaries. The STFT is defined as:

$$STFT = \frac{sum of the followup time for pending patients at the current dose}{length of the DLT observation period}$$

Although the TITE-BOIN design allows sequential enrollment even though the previous participants have not completed the DLT observation period, a suspension rule to hold off the dose escalation/de-escalation decision is applied if more than 50% of participants have not finished the DLT assessment at the current dose level from the safety perspective.

TABLE 3
Dose Escalation/De-escalation Rule for the TITE-BOIN Design
Corresponding to the Cumulative Number of Participants (3, 6, or 9)

| No. | No. | No. | | | |
|---|---|---|---|---|---|
| Evaluable | DLTs | Pending | Escalation | Stay | De-escalation |
| 3 | 0 | ≤1 | Yes | No | No |
| 3 | 0 | ≥2 | Suspend | Suspend | Suspend |
| 3 | 1 | 0 | No | Yes | No |
| 3 | 1 | 1 | No | STFT > 0.88 | STFT ≤ 0.88 |
| 3 | 1 | ≥2 | Suspend | Suspend | Suspend |
| 3 | 2 | ≤1 | No | No | Yes |
| 3 | 3 | 0 | No | No | Yes & |
| | | | | | Eliminate |
| 6 | 0 | ≤3 | Yes | No | No |
| 6 | 0 | ≥4 | Suspend | Suspend | Suspend |
| 6 | 1 | ≤1 | Yes | No | No |
| 6 | 1 | 2 | STFT ≥ | STFT < 0.6 | No |
| | | | 0.6 | | |
| 6 | 1 | 3 | STFT ≥ | STFT < 1.96 | No |
| | | | 1.96 | | |
| 6 | 1 | ≥4 | Suspend | Suspend | Suspend |
| 6 | 2 | 0 | No | Yes | No |
| 6 | 2 | 1 | No | STFT > 0.73 | STFT ≤ 0.73 |
| 6 | 2 | 2 | No | STFT > 1.8 | STFT ≤ 1.8 |
| 6 | 2 | 3 | No | STFT > 2.87 | STFT ≤ 2.87 |
| 6 | 2 | ≥4 | Suspend | Suspend | Suspend |
| 6 | 3 | ≤3 | No | No | Yes |
| 6 | ≥4 | ≤2 | No | No | Yes & |
| | | | | | Eliminate |
| 9 | 0 | ≤4 | Yes | No | No |
| 9 | 0 | ≥5 | Suspend | Suspend | Suspend |
| 9 | 1 | ≤4 | Yes | No | No |
| 9 | 1 | ≥5 | Suspend | Suspend | Suspend |
| 9 | 2 | 0 | Yes | No | No |
| 9 | 2 | 1 | STFT ≥ | STFT < 0.59 | No |
| | | | 0.59 | | |
| 9 | 2 | 2 | STFT ≥ | STFT < 1.65 | No |
| | | | 1.65 | | |
| 9 | 2 | 3 | STFT ≥ | STFT < 2.71 | No |
| | | | 2.71 | | |
| 9 | 2 | 4 | STFT ≥ | STFT < 3.77 | No |
| | | | 3.77 | | |
| 9 | 2 | ≥5 | Suspend | Suspend | Suspend |
| 9 | 3 | 0 | No | Yes | No |
| 9 | 3 | 1 | No | STFT > 0.58 | STFT ≤ 0.58 |
| 9 | 3 | 2 | No | STFT > 1.65 | STFT ≤ 1.65 |
| 9 | 3 | 3 | No | STFT > 2.72 | STFT ≤ 2.72 |
| 9 | 3 | 4 | No | STFT > 3.79 | STFT ≤ 3.79 |
| 9 | 3 | ≥5 | Suspend | Suspend | Suspend |
| 9 | 4 | ≤5 | No | No | Yes |
| 9 | ≥5 | ≤4 | No | No | Yes & |
| | | | | | Eliminate |

Note:
When a dose is eliminated, all higher doses should also be eliminated.
DLT: dose-limiting toxicity;
STFT: standardized total follow-up time

### Maximum Tolerated Dose and Recommended Phase 2 Dose

Maximum tolerated dose (MTD) is defined as the dose level at which the DLT rate estimated using an isotonic regression is closest to the target DLT rate of 3000.

The sponsor will determine the RP2D of Compound I taking into consideration available data on safety, pharmacodynamics and efficacy of Compound I. The RP2D) will not exceed the MTD. A minimum number of 6 participants must be enrolled at the dose level used to determine the MTD and/or RPD.

### Dose Limiting Toxicities

A DLT is defined as any of the following adverse events (AEs) (graded using National Cancer Institute Common Terminology Criteria for Adverse Events [NCI-CTCAE] version 5.0 that the investigator (or sponsor) cannot clearly attribute to a cause other than study drug:
Grade 4 neutropenia
Grade ≥3 febrile neutropenia
Grade 4 thrombocytopenia
Grade 3 thrombocytopenia accompanied by bleeding that requires any transfusion
Grade 4 anemia
Grade 3 anemia requiring transfusion
Grade ≥3 non-hematological AE (excluding grade 3 elevation of amylase and lipase not associated with clinical or radiographic evidence of pancreatitis and asymptomatic grade 3 hypophosphatemia)
Grade ≥2 pneumonitis
Grade ≥2 encephalopathy, meningitis, or motor or sensory neuropathy
Aspartate aminotransferase (AST) or alanine aminotransferase (ALT) >5× upper limit of normal (ULN; grade ≥3) without liver metastases
AST or ALT >8×ULN in participants with liver metastases
AST or ALT >3×ULN and total bilirubin >2×ULN (in participants with Gilbert syndrome: AST or ALT >3×ULN and direct bilirubin >1.5×ULN)
Total bilirubin >3×ULN (grade ≥3)
Guillain-Barre syndrome or myasthenic syndrome/myasthenia gravis
Unresolved AE grade ≥2 that results in prolonged delay (>14 days) in initiating cycle 2
Any toxicity that causes the participant to discontinue treatment during cycle 1
Grade 5 toxicity
Any grade 4 immune-related adverse event (irAE)
Grade 3 irAE or irAE for which corticosteroid dose is not able to be reduced within a reasonable timeframe (e.g., recurrent grade 3 immune-mediated reactions that require systemic immunosuppressive treatment; inability to reduce corticosteroid dose to 10 mg or less of prednisone or equivalent per day within 12 weeks of initiating steroids)
Liver function test (LFT) elevations lasting >7 days that are considered to be clinically significant and at least possibly related to Compound I
Grade 4 fatigue and anorexia of any duration and grade 3 fatigue or anorexia with duration >7 days should be considered a DLT

The following AEs will not be considered DLTs:
Electrolyte abnormalities that are not associated with clinical sequelae or deemed not clinically significant and corrected with appropriate management or supplementation within 72 hours of onset
Alopecia
Grade 3 fatigue or anorexia lasting ≤7 days
Grade 3 LFT elevations that resolve to ≤grade 1 within 7 days
irAEs that resolve to ≤grade 1 within 72 hours of onset after starting treatment, including corticosteroids

Confirmation of DLTs will be made by the Dose Escalation and Safety Committee (DESC). Participants who are tolerating study drug at a dose level that is being reviewed due to the occurrence of DLTs in another participant will not be automatically precluded from continuing dosing during the safety review, and will be allowed to continue dosing for as long as tolerated unless directed otherwise as a result of the safety review by the DESC.

### Replacement of Participants in Dose Escalation Cohorts

A participant without a DLT who receives <80% of the intended dose (cohort dose level) during cycle 1 (e.g., misses >4 daily doses in cycle 1) or does not complete cycle 1 evaluation for a reason other than DLT (e.g., consent withdrawal) will not be DLT evaluable and will be replaced by another participant in the dose level.

### Part 2: Dose Expansion

### Stage 1—Response-Triggered Expansion Cohorts

As the dose escalation cohorts proceed in Part 1, additional participants may be enrolled in a response expansion cohort in Stage 1 of Dose Expansion. If a confirmed response (partial response [PR] or complete response [CR]) based on a local assessment of Immune Response Evaluation Criteria in Solid Tumors (iRECIST) occurs in a dose escalation cohort, a tumor-specific expansion cohort may be opened in that tumor type at a dose level that has been cleared and deemed tolerable by the DESC. Up to 20 participants will initially be enrolled in this Part 2—Stage 1 response-triggered expansion cohort. Participants will be evaluated for DLTs during cycle 1 in expansion cohorts for continued safety evaluation.

### Stage 1—RP2D Expansion Cohorts

RP2D expansion cohorts will be enrolled following completion of dose escalation (Part 1) and determination of the MTD/RP2D. Up to approximately 20 participants will initially be enrolled in the RP2D expansion cohorts (i.e., metastatic melanoma and NSCLC). Participants will be evaluated for DLTs during cycle 1 in expansion cohorts. The RP2D expansion cohorts will assess safety and tolerability and preliminary efficacy of Compound I at the RP2D.

### Stage 2

Stage 2 may be opened for specific tumor type(s) based on observed pharmacokinetic and anti-tumor activity from Stage 1.

Dose Optimization: One tumor type from the response-triggered/RP2D expansion cohorts in Stage 1 will be selected for Dose Optimization, which will include a randomization of up to 40 participants between 2 different dose levels: Dose A (RP2D or response-triggered dose) and Dose B. The choice of tumor type (e.g., Tumor Type 1) and the selection of Dose B, will be made based on the available clinical data from the dose escalation and dose expansion cohorts.

If initial 20 participants with Dose B meet the criteria, up to additional participants may be enrolled for further evaluation to reach a total of 40 participants treated with Dose B.

Response-triggered and RP2D Dose Expansion Cohort(s): Up to 20 additional participants may be enrolled in each additional tumor type that meets the criteria for Stage 2 (e.g., Tumor Types 2 and 3).

### Efficacy Monitoring in Expansion Cohorts (Stages 1 and 2)

If at least 1 response based on a local assessment of iRECIST is observed in the first 20 participants of a response or RP2D triggered expansion cohort from Stage 1 and the dose is confirmed tolerable, then up to approximately 20 additional participants with that specific tumor type may be enrolled, respectively. The Bayesian optimal phase 2 (BOP2) design [Zhou et al., 2017] will be used in the study to evaluate the efficacy in terms of objective response rate in each tumor type for the selected dose level. The optimized stopping boundaries are provided in the table below. The BOP2 design declares the dose level is acceptable for the tumor type from the efficacy perspective if 3 or more responders are observed in each tumor type for the selected dose level in the 40 participants at the final analysis. The final decision on the addition of participants to any expansion cohort will be made by the sponsor following confirmation of dose tolerability by the DESC.

### Optimized Efficacy Stopping Boundaries:

| No. participants treated | Stop if no. responses≤ |
|---|---|
| 20 | 0 |
| 40 | 2 |

With assumption of the efficacious overall response rate (ORR) is 10% and the inefficacious ORR is 2%, the statistical power would be approximately 73.8% while controlling the type I error rate at 4.1%.

### Replacement of Participants in Expansion Cohorts

If a participant in an expansion cohort is not response evaluable (defined as the full analysis set), then an additional participant may be enrolled in that cohort based on sponsor discretion.

### Dose Escalation and Safety Committee

A Dose Escalation and Safety Committee (DESC) consisting of sponsor representatives and investigators will convene once a dose level cohort completes the DLT observation period and the data are available for review. The DESC's decision on the dose level for the next cohort will be guided by the TITE-BOIN, which is based on DLTs observed at each dose level.

While safety data from the DLT observation period in the escalation cohorts are the minimum safety data needed for the committee meeting, all available safety findings, including those occurring after the designated DLT observation period that meet DLT criteria (“delayed DLT”), will be considered by the DESC. The DESC will assess whether a longer DLT observation period is warranted, based on emerging data. Additionally, the DESC may choose a more conservative dosing decision than indicated by the TITE-BOIN, based on evaluation of the safety data and available pharmacokinetic data.

The DESC will also review the aggregate safety data from the expansion cohorts. Based on the available data, the DESC may choose more conservative stopping rules for the expansion cohorts than those outlined in the Sample Size Justification section below.

Following the DESC review, the sponsor will determine the final RP2D based on the efficacy and safety in the dose optimization expansion cohort.

### Study Design and Dose Rationale: Scientific Rationale for Study Design

This is a phase 1/2, open-label, multi-center, multiple-dose, dose escalation and expansion study of Compound I. Anti-tumor efficacy of Compound I was demonstrated in anti-PD-1 poor-responsive syngeneic mouse models as well as in responsive models. Blinding is not required to reduce bias because the primary objectives are safety and tolerability, which will be measured with objective endpoints. Therefore, an open-label design will be used. As this is a FIH study, the primary endpoints of safety, tolerability, MTD and RP2Ds are clinically relevant to the evaluation of this product. The primary endpoint of safety evaluates the AEs the participant experiences and therefore constitutes a clinically meaningful effect. The eligibility requirements and safety monitoring are designed to minimize risks to the participants given the nonclinical safety assessments that have been performed, which suggest risk of effect on body weight, gastrointestinal tract, electrocardiogram (ECG) parameters, hemodynamic and body temperature, lymphohematopoietic system and reproductive toxicity. Participants with human immunodeficiency virus (HIV) will be excluded since a compromised immune system may affect the immune response induced by Compound I. Overall, the risks associated with participation in this clinical study of Compound I are considered to be acceptable for this population of participants with locally advanced or metastatic solid tumor malignancies (no limit to the number of prior treatment regimens) who have progressed on or are no longer eligible for standard therapy.

### Study Design and Dose Rationale: Dose Rationale

Compound I has been tested in numerous preclinical studies. Data from the 4-week repeated dose toxicity study in rats and 4-week repeated dose toxicity study in cynomolgus monkeys determined STD10 in rats to be 30 mg/kg/day and the HNSTD in cynomolgus monkeys to be 3 mg/kg/day. Human equivalent doses based on body surface area conversion (conversion factors: 0.16 for rat, 0.32 for monkey) of the STD10 in rats and the HNSTD in cynomolgus monkeys are 4.8 mg/kg and 0.96 mg/kg, respectively. For a person of 60 kg body weight, one tenth of the rat STD10 is 28.8 mg (4.8 mg/kg× 1/10×60 kg) and one sixth of the HNSTD is 9.6 mg (0.96 mg/kg×⅙×60 kg). Cynomolgus monkey is the more sensitive species. Therefore, based on cynomolgus monkey HNSTD, the starting dose in human is judged to be 9.6 mg/participant once daily (QD) given orally (actual starting dose will be 10 mg based on 10 mg tablet strength).

### Study Design and Dose Rationale: End of Study Definition

The end of the study is defined as the last visit or scheduled procedure shown in Schedule of Assessments (FIG. 4) for the last participant in the study.

### Study Population

All screening assessments must be completed and reviewed to confirm the potential participant meets all eligibility criteria. Prospective approval of protocol deviations to eligibility criteria (also known as protocol waivers or exemptions) is not permitted.

### Study Population: Inclusion Criteria

Participant sex will be defined as male or female as assigned at birth. Participants are eligible for participation in the study if all of the following apply:
1. Institutional Review Board (IRB)/Independent Ethics Committee (IEC) approved written informed consent and privacy language as per national regulations (e.g., Health Insurance Portability and Accountability Act authorization for US study sites) must be obtained from the participant prior to any study-related procedures (including withdrawal of prohibited medication, if applicable).
2. Participant is considered an adult according to local regulation at the time of signing the informed consent form (ICF).
3. Participant has locally advanced (unresectable) or metastatic solid tumor malignancy which is confirmed by available pathology records or current biopsy.
4. Participant has at least 1 measurable lesion per Response Evaluation Criteria in Solid Tumors (RECIST) v1.1. Lesions situated in a previously irradiated area are considered measurable if progression has been demonstrated in such lesions.
5. Participant has progressed after receiving all standard approved therapies or is no longer eligible for standard therapy (no limit to the number of prior treatment regimens).
6. Participant has an Eastern Cooperative Oncology Group (ECOG) Performance Status of 0, 1 or 2.
7. Participant's last dose of prior antineoplastic therapy, including any immunotherapy, was at least 21 days prior to initiation of study drug administration. A participant with solid tumors that have a neurotropic receptor tyrosine kinase (NTRK) gene fusion without a known acquired resistance mutation or a participant with epidermal growth factor receptor (EGFR) or anaplastic lymphomas kinase (ALK) mutation-positive NSCLC is allowed to remain on NTRK inhibitors or EGFR tyrosine kinase inhibitor (TKI) or ALK inhibitor therapy until 4 days prior to the first dose of investigational product (IP).
8. Participants who have received radiotherapy must have completed this therapy (including stereotactic radiosurgery) at least 2 weeks prior to the first dose of IP.
9. Participant's adverse events (excluding alopecia) from prior therapy have improved to grade 1 or baseline within 14 days prior to the first dose of IP. Note: Participants with type 1 diabetes mellitus, endocrinopathies stably maintained on appropriate replacement therapy, or skin disorders (e.g., vitiligo, psoriasis, or alopecia) not requiring systemic treatment are allowed.
10. Participant has adequate organ function prior to start of study treatment as indicated by the following laboratory values. If a participant has received a recent blood transfusion, the laboratory tests must be obtained ≥2 weeks after any blood transfusion:

| | |
|---|---|
| Parameter | Laboratory Value |

Hematological

| ANC | ≥1500/μL | |
|---|---|---|
| Platelets | ≥100,000/μL | |
| Hemoglobin | ≥9 | g/dL |

Renal

| | |
|---|---|
| Creatinine | Either: |
| | a) ≤ institutional ULN, OR |
| | b) CLcr ≥60 mL/min (calculated by Cockroft-Gault |
| | equation) |

Hepatic

| | |
|---|---|
| Total bilirubin | Either: |
| | a) ≤1.5 × ULN; or |
| | b) Direct bilirubin ≤ ULN and total bilirubin <3 × |
| | ULN (for participants with Gilbert's syndrome) |
| AST (SGOT) and | ≤2.5 × ULN without liver metastases (or ≤5 × |
| ALT (SGPT) | ULN if liver metastases are present) |

Metabolic

| | | |
|---|---|---|
| Serum potassium | ≥3.4 | mEq/L |
| Serum magnesium | ≥1.7 | mg/dL |
| Serum ionized | ≥4.7 | mg/dL |
| calcium | | |

ALT: alanine aminotransferase;
ANC: absolute neutrophil count;
AST: aspartate aminotransferase;
CLcr: creatinine clearance;
SGOT: serum glutamic oxaloacetic transaminase;
SGPT: serum glutamic pyruvic transaminase;
ULN: upper limit of normal

11. Participant has activated partial thromboplastin time (aPTT) and international normalized ratio (INR)≤1.5×ULN and is not receiving anticoagulation.
12. Female participant is not pregnant (see Appendix 2) and at least one of the following conditions apply:
   a. Not a woman of childbearing potential (see Appendix 2)
   b. Woman of childbearing potential who agrees to follow the contraceptive guidance (see Appendix 2) from the time of informed consent through at least 30 days after final study intervention administration.
13. Female participant must agree not to breastfeed starting at screening and throughout the study period and for 30 days after final study intervention administration.
14. Female participant must not donate ova starting at first dose of study intervention and throughout the study period and for 30 days after final study intervention administration.
15. Male participant with female partner(s) of childbearing potential (including breastfeeding partner) must agree to use contraception (see Appendix 2) throughout the treatment period and for 30 days after final study intervention administration.
16. Male participant must not donate sperm during the treatment period and for 30 days after final study intervention administration.
17. Male participant with pregnant partner(s) must agree to remain abstinent or use a condom for the duration of the pregnancy throughout the study period and for 30 days after final study intervention administration.
18. Participant agrees not to participate in another interventional study while receiving study intervention in the present study.

### Study Population: Exclusion Criteria

Participant will be excluded from participation in the study if any of the following apply:
1. Participant has received any investigational therapy (other than an EGFR TKI in a participant with EGFR-activating mutations, ALK inhibitor in a participant with an ALK mutation or NTRK inhibitor in a participant with solid tumors that have a NTRK gene fusion without a known acquired resistance mutation) within 21 days or 5 half-lives, whichever is shorter, prior to the first dose of IP.
2. Participant requires or has received systemic steroid therapy or any other immunosuppressive therapy within 14 days prior to the first dose of study intervention. Participants using a physiologic replacement dose of hydrocortisone or its equivalent (defined as up to 30 mg per day of hydrocortisone and up to 10 mg prednisone) are allowed.
3. Participant requires strong or moderate CYP2D6 inhibitors (e.g., bupropion, fluoxetine, paroxetine, duloxetine, abiraterone) during the study.
4. Participant requires strong CYP3A4 inhibitors (e.g. boceprevir, clarithromycin, cobicistat, indinavir, itraconazole, nefazodone, nelfinavir, posaconazole, ritonavir, saquinavir, telaprevir, telithromycin, voriconazole) during the study.
5. Participant has symptomatic central nervous system (CNS) metastases or participant has evidence of unstable CNS metastases even if asymptomatic (e.g., progression on scans). Participants with previously treated CNS metastases are eligible if they are clinically stable and have no evidence of CNS progression by imaging for at least 4 weeks prior to start of study treatment and are not requiring immunosuppressive doses of systemic steroids (>30 mg per day of hydrocortisone or >10 mg per day of prednisone or equivalent) for no longer than 2 weeks.
6. Participant has an active autoimmune disease. Participants with type 1 diabetes mellitus, endocrinopathies stably maintained on appropriate replacement therapy, or skin disorders (e.g., vitiligo, psoriasis, or alopecia) not requiring systemic treatment are allowed.
7. Participant was discontinued from prior immunomodulatory therapy due to a grade ≥3 toxicity that was mechanistically related (e.g., immune-related) to the agent in the judgment of the investigator.
8. Participant is known to have human immunodeficiency virus (HIV) infection. However, participants with HIV with CD4+ T-cell counts ≥350 cells/μL and no history of AIDS-defining opportunistic infections within the past 6 months are eligible. NOTE: Screening for HIV infection should be conducted per local requirements.
9. Participant has any of the following per screening serology test:
   a. Hepatitis A virus (HAV) antibodies (immunoglobulin M [IgM])
   b. Positive hepatitis B surface antigen (HBsAg) or detectable hepatitis B DNA. Participants with negative HBsAg, positive hepatitis B core antibody (anti-HBc) and negative hepatitis B surface antibody (anti-HBs) are eligible if hepatitis B DNA is undetectable
   c. Hepatitis C virus (HCV) antibodies unless HCV RNA is undetectable
10. Participant has received a live vaccine against infectious diseases within 28 days prior to initiation of the first dose of study intervention.
11. Participant has a history of drug-induced pneumonitis (interstitial lung disease) or currently has pneumonitis or a prior history of ILD or noninfectious pneumonitis requiring high-dose glucocorticoids, whether resolved or not.
12. Participant has an infection requiring systemic therapy within 14 days prior to the first dose of study intervention.
13. Participant has received a prior allogenic bone marrow or solid organ transplant.
14. Participant is expected to require another form of antineoplastic therapy while on study treatment.
15. Participant has had a myocardial infarction or unstable angina within 6 months prior to the start of study intervention or currently has an uncontrolled illness including, but not limited to symptomatic congestive heart failure, clinically significant cardiac disease, unstable angina pectoris, cardiac arrhythmia, or psychiatric illness/social situations that would limit compliance with study requirements.
16. Participant has inadequately controlled hypertension (defined as systolic blood pressure >150 and/or diastolic blood pressure >100 mmHg on antihypertensive medications).
17. Participant has a corrected QT interval (Single ECG) using Fridericia's formula (QTcF) >450 ms (for male and female participants) during screening. ECGs will be performed in triplicate during screening.
18. Participant has another malignancy requiring active therapy, except for locally curable malignancies, such as basal or squamous cell skin cancer, superficial bladder cancer or carcinoma in situ of the cervix or breast.
19. Participant has had a major surgical procedure and has not completely recovered within 28 days prior to the first dose of study intervention.
20. Participant has a history of bleeding diathesis.
21. Participant requires the use of any anticoagulation therapy.
22. Participant has any condition, which, in the investigator's opinion, makes the participant unsuitable for study participation.
23. Participant has been previously treated with a DGK inhibitor.
24. Participant has a known or suspected hypersensitivity to Compound I, or any components of the formulation used.

### Study Population: Screen Failures

A screen failure is defined as a potential participant who signed the ICF, but did not meet 1 or more criteria required for participation in the study and was not enrolled.

For screen failures, the demographic data, date of signing the ICF, inclusion and exclusion criteria, AEs up to the time of screen failure and reason for screen failure will be collected in the eCRF

### Screen Failures: Rescreening

Results of screening assessments that do not meet the parameters required by eligibility criteria (e.g., clinical laboratory tests, vital signs, physical examination, ECG, etc.) may be repeated once within the 28-day screening period without the need to register the participant as a screen failure. If the participant meets exclusion criteria that cannot resolve during the screening period, or more than 28 days elapse from the date of signing the ICF, the participant must be documented as a screen failure. In order to re-screen after prior screen failure, a new ICF must be signed and the participant entered into screening with a new participant identification number. Rescreening is only allowed once for an individual participant.

If a participant's electrolytes do not meet the eligibility criteria, testing may be repeated once within the 28-day screening period without screen failing.

### Study Intervention and Concomitant Therapy

Study interventions (shown in Table 4) are all pre-specified, investigational and non-investigational medicinal products, medical devices, vaccines and other interventions (e.g., surgical and behavioral) intended to be administered to the study participants during the study conduct.

TABLE 4
Study Intervention(s) Administered

| | |
|---|---|
| Intervention Name | Compound I tablets 10 mg |
| | Round, light yellowish red film-coated tablet |
| | Compound I tablets 50 mg |
| | Oval, light yellowish red film-coated tablet |
| Type | drug |
| Dose Formulation | tablet |
| Unit Dose | 10, 50 mg |
| Strength(s) | |
| Dosage Level(s) | daily dosing until disease progression |
| Route of | oral |
| Administration | |
| Use | experimental |
| IMP and | IMP |
| NIMP/AxMP | |
| Sourcing | Provided centrally by the sponsor or locally by the study site, |
| | subsidiary, or designee. |
| Packaging and | Study intervention will be provided in container. Each container |
| Labeling | will be labeled as required per country requirement. |
| | Compound I Tablets 10 mg and 50 mg are packed in a HDPE bottle |
| | and PP cap with desiccant (Silica gel 2 g)/aluminum pouch (30 |
| | tablets per bottle) |

AxMP: auxiliary medicinal product;
HDPE: high-density polyethylene;
IMP: Investigational Medicinal Product;
NIMP: noninvestigational medicinal product;
PP: polypropylene

### Study Intervention and Concomitant Therapy: Preparation/Handling/Storage/Accountability: Packaging and Labeling

All sponsor-provided study intervention used in this study will be prepared, packaged and labeled under the responsibility of qualified personnel in accordance with SOPs, current GMP guidelines, ICH GCP guidelines and applicable local laws/regulations.

Each bottle and pouch will bear a label conforming to regulatory guidelines, GMP and local laws and regulations that identifies the contents as investigational drug.

Refer to the pharmacy manual for detailed information regarding packaging and labeling of the study intervention.

### Study Intervention and Concomitant Therapy: Preparation/Handling/Storage/Accountability: Handling, Storage and Accountability

The investigator or designee must confirm appropriate conditions have been maintained during transit for all study intervention received and any discrepancies are reported and resolved before use of the study intervention.

Only participants enrolled in the study may receive study intervention and only authorized study site personnel may supply, prepare, or administer study intervention. Only study intervention with appropriate expiry/retest dating may be dispensed.

All study intervention must be stored in a secure, environmentally controlled and monitored (manual or automated) area in accordance with the labeled storage conditions and access must be limited to the investigator and authorized study site personnel.

The investigator, institution or the head of the medical institution (where applicable) or authorized site staff is responsible for accountability, reconciliation and record maintenance (i.e., receipt, reconciliation and final disposition records including Certificate of Destruction or equivalent).

The sponsor is responsible for providing further guidance and instruction on final disposition of used and unused study intervention in the pharmacy manual.

Refer to the pharmacy manual for detailed information regarding handling, storage and accountability of the study intervention.

### Study Intervention and Concomitant Therapy: Assignment to Study Intervention

This is an open-label study. Participant enrollment and dispensation of IP will be performed via the interactive response technology (IRT) system. Specific TRT procedures will be described in the respective study manual.

### Study Intervention and Concomitant Therapy: Study Intervention Compliance

Participant compliance with IP will be assessed at each visit. Compliance will be assessed by drug accountability (i.e., counting returned tablets and/or direct questioning). Deviations from the prescribed dose regimen will be recorded.

If compliance is less than 80%, the investigator or designee is to counsel the participant and ensure steps are taken to improve compliance. Participants without a DLT who receive <80% of the intended dose during cycle 1 (e.g., misses >4 daily doses in cycle 1), or do not complete cycle 1 evaluation for a reason other than DLT (e.g., consent withdrawal) will not be DLT evaluable and may be replaced by another participant in the dose level.

### Study Intervention and Concomitant Therapy: Dose Modification

Dose modifications of Compound I are not allowed at the individual participant level without prior consultation with the sponsor's medical monitor. For the escalation or expansion cohort participants, study drug treatment may be interrupted for any AE, laboratory abnormality or intercurrent illness that in the judgement of the investigator warrants delaying dosing of study drug treatment. Dose reductions are not allowed in the escalation stage of the study unless the dose level has been reviewed and approved by the DESC. Dose reductions are not allowed in the dose optimization cohorts in the dose expansion stage of the study. Cycle days will continue to be counted regardless of whether the dose is taken, in order to keep visits on a consistent schedule.

### Toxicities Requiring Treatment Interruption of Compound I

Compound I treatment will be withheld for the following toxicities if there is a reasonable possibility that the event may have been caused by Compound I.
Grade ≥3 non-hematologic AEs
Grade 4 neutropenia or grade ≥3 febrile neutropenia
Grade ≥3 anemia requiring red blood cell transfusion
Grade ≥3 thrombocytopenia accompanied by bleeding that requires transfusion (platelets or red blood cells)
In participants without liver metastases:
   AST or ALT >5×ULN OR
   AST or ALT >3×ULN and TBL >2×ULN (in participant with Gilbert syndrome: AST or ALT >3×ULN and direct bilirubin >1.5×ULN)
In participants with liver metastases: AST or ALT >8×ULN
TBL >3×ULN (grade ≥3)
Grade 3 or higher cytokine release syndrome (CRS) (graded using American Society for Transplantation and Cellular Therapy [ASTCT] consensus grading; see Appendix 10, Algorithm for Management of Cytokine Release Syndrome)
Any grade 2 or higher irAE
Any AE, laboratory abnormality or intercurrent illness that in the judgment of the investigator warrants delaying dosing of study drug treatment.

### Criteria for Resuming Treatment

Study intervention dosing may be interrupted for up to 4 weeks to allow recovery of toxicity requiring interruption. After discussion with the medical monitor, study intervention may be resumed at the same or a reduced dose (one dose level down from the assigned dose) if AEs have improved to grade 0 or 1, and steroid treatment tapered to physiological replacement doses (≤30 mg hydrocortisone or ≤10 mg prednisone or equivalent), and do not meet the study treatment discontinuation criteria.

For a list of toxicities requiring permanent discontinuation of Compound I, see Discontinuation of Individual Participant(s) from Study Treatment below. For management algorithms of any potential immune-related toxicities see Appendix 9.

### The Following Guideline Will be Followed for Participants Experiencing CRS:

If grade 2 CRS is experienced, resume study intervention upon resolution of CRS to grade ≤1.
If grade 3 CRS is experienced, resume study intervention upon resolution of CRS to grade ≤1 with the following restrictions:
   The dose of study intervention should be at least one dose level lower than the current dose and will not be escalated if grade 3 or higher CRS is experienced at the reduced dose.
   If the reduced dose is tolerated for a full cycle with no grade 3 CRS, the participant may receive the dose in which the grade 3 CRS occurred.
Participants experiencing grade 4 CRS or recurrent grade ≥3 CRS will be discontinued (see Discontinuation of Individual Participant(s) from Study Treatment, below.)

The grading of CRS will be per the ASTCT Consensus Grading of CRS criteria requiring assessment of fever, hypotension, and hypoxia. For treatment guidelines for CRS, refer to Appendix 10 (Algorithm for Management of Cytokine Release Syndrome) and Appendix 11 (Algorithm for Management of Immune-effector Cell-associated Neurotoxicity Syndrome).

### Intra-Participant Dose Escalation

For participants who did not experience a DLT and have completed at least 2 cycles of treatment, intra-participant dose escalation is allowed after a higher dose level has been cleared. These participants may subsequently increase to a higher dose level again after the higher dose level has cleared (no limit on the number of escalations). The investigator must notify the medical monitor before proceeding with intra-participant dose escalation.

Study Intervention and Concomitant Therapy: Continued Access to Study Intervention after the End of the Study

Compound I will not be made available after conclusion of the study.

### Study Intervention and Concomitant Therapy: Treatment of Overdose

In the event of suspected Compound I overdose, the participant should receive supportive care and monitoring. The medical monitor/expert should be contacted, as applicable.

Refer to Medication Error, Overdose and “Off-label Use” section below for reporting requirements for suspected overdose or other medication error.

### Study Intervention and Concomitant Therapy: Prior and Concomitant Therapy

Investigational agents: The use of other investigational agents is not allowed during study treatment.

Steroids and other immunosuppressive therapy: The use of immunosuppressive agents and immunosuppressive doses of systemic steroids (>30 mg per day of hydrocortisone or

>10 mg per day of prednisone or equivalent) is not allowed during study treatment unless needed to manage adverse events related to study treatment. The use of topical, ocular, intra-articular, intranasal, and inhalational corticosteroids (with minimal systemic absorption) is allowed. Physiologic replacement doses of systemic corticosteroids (≤30 mg per day of hydrocortisone or ≤10 mg per day of prednisone or equivalent) are permitted. Corticosteroids for prophylaxis (e.g., contrast dye allergy) or for brief treatment of conditions not related to study treatment (e.g., delayed-type hypersensitivity reaction caused by a contact allergen) is also allowed.

Other anti-cancer treatment: The use of other anti-cancer therapy (e.g., chemotherapy, hormonal therapy, immunotherapy, radiotherapy, biological therapy, targeted therapy) is not allowed during study treatment.

Palliative (limited field) radiation therapy for bone metastases is allowed. Study treatment should be interrupted during radiation therapy. The use of bisphosphonates and receptor activator of nuclear factor kappa-B ligand inhibitors for bone metastases is allowed if initiated prior to screening. Surgical treatment of isolated or symptomatic lesions for palliation or curative management is also allowed beyond cycle 4. Study treatment should be interrupted until the lesion is healed.

Vaccinations: Live vaccines are not allowed while the participant is receiving study treatment and for 14 days after the last dose of study treatment. Non-live vaccines (e.g., COVID-19 vaccines) are permitted. Investigators are highly recommended to consider any confounding variables relating to the potential irAEs for Compound I and vaccines, especially during the DLT window. Investigators are also strongly recommended to avoid overlapping toxicities that might compromise the safety and tolerability of either treatment. Participants entering the trial who are candidates for a COVID-19 vaccine are encouraged to receive the first dose while undergoing screening procedures.

Other prohibited medications/therapies: Herbal supplements that have the potential to cause prolongation of the QTc interval or other possible toxic/undesirable effects are not allowed during study treatment.

Any over the counter medications with the potential to cause prolongation of the QTc interval are not allowed during study treatment.

Medications that affect the coagulation cascade such as vitamin K antagonists, heparins and direct thrombin inhibitors or the use of factor Xa inhibitors are not allowed during study treatment.

The use of strong and moderate CYP2D6 inhibitors (e.g., bupropion, fluoxetine, paroxetine, duloxetine, abiraterone) is not allowed during study treatment.

The use of strong CYP3A4 inhibitors (e.g., boceprevir, clarithromycin, cobicistat, indinavir, itraconazole, nefazodone, nelfinavir, posaconazole, ritonavir, saquinavir, telaprevir, telithromycin, voriconazole) is not allowed during study treatment.

All concomitant treatments will be recorded in the eCRF.

### Study Procedures and Assessments

Study procedures and their timing are summarized in the Schedule of Assessments (FIG. 4). Adherence to the study design requirements, including those specified in the Schedule of Assessments, is essential and required for study conduct. Prospective protocol waivers or exemptions are not allowed.

Any change, divergence or departure from the study design or procedures identified in the protocol is considered a protocol deviation. All deviations from the protocol are to be recorded.

All screening evaluations must be completed and reviewed to confirm that potential participants meet all eligibility criteria. The investigator will maintain a screening log to record details of all participants screened and to confirm eligibility or record reasons for screening failure, as applicable.

Procedures conducted as part of the participant's routine clinical management (e.g., imaging, blood count) and obtained before signing of the ICF may be utilized for screening or baseline purposes provided the procedures met the protocol-specified criteria and were performed within the time frame defined in the Schedule of Assessments (FIG. 4).

In the event of a significant study-continuity issue (e.g., caused by a pandemic), alternate strategies for participant visits, assessments, medication distribution and monitoring may be implemented by the sponsor or the investigator, as per local health authority/ethics requirements. These alternate strategies are described in Appendix 7.

### Study Procedures and Assessments: Efficacy Assessments

Planned time points for all efficacy assessments are provided in the Schedule of Assessments (FIG. 4).

Disease response and progression will be evaluated in this study using the RECIST 1.1 and iRECIST criteria as assessed by the investigator.

Tumor assessments will be performed at screening and every 9 weeks (±1 week) from cycle 1 day 1 (C1D1) until “immune” confirmed progressive disease (iCPD). Imaging should include chest, abdomen and pelvis, as well as any other anatomical region appropriate for the participant's disease. The assessment will include tumor measurements for target lesions, non-target lesions and assessment for any new lesions. An overall assessment will be characterized for that time point evaluation.

Computed tomography (CT) or magnetic resonance imaging (MRI) scans are preferred for this study and to ensure comparability; the same technique (CT/MRI) used at screening should be utilized throughout the study. The same method should be employed and assessed by the same individual on each occasion, when possible. Imaging should include chest, abdomen and pelvis, as well as any other anatomical region appropriate for the participant's disease.

Imaging should be done every 9 weeks from C1D1 regardless of treatment interruption or delays and will continue through Safety Follow-up. Scans performed prior to informed consent as standard of care are acceptable as screening scans if done within 28 days prior to C1D1. If a biopsy of a target lesion is performed, then the baseline scan should be repeated, if possible.

Confirmatory scans for CR or PR should be done at least 4 weeks after the date of the scan in which CR or PR was first observed. Confirmatory scans for progressive disease must occur at least 4 weeks after the date of the scan in which progressive disease was first observed, but no longer than 8 weeks.

All images performed as part of disease assessment will be read locally. However, copies of all scans from participants in expansion cohorts will be submitted to a central vendor and stored to be available for future analysis if warranted. Additional information will be provided per Imaging Manuals.

### Study Procedures and Assessments: Safety Assessments

Planned time points for all safety assessments are provided in the Schedule of Assessments (FIG. 4).

### Study Procedures and Assessments: Safety Assessments: Laboratory Assessments

See Appendix 5 for the list of clinical laboratory tests to be performed and refer to Schedule of Assessments (FIG. 4) for timing and frequency.

The investigator must review the laboratory report, document this review, and record any clinically significant changes occurring during the study as an AE. The laboratory reports must be filed with the source documents.

Clinical significance of out-of-range laboratory findings is to be determined and documented by the investigator or sub-investigator who is a qualified physician. Abnormal laboratory findings associated with the underlying disease are not considered clinically significant unless judged by the investigator to be more severe than expected for the participant's condition.

### Study Procedures and Assessments: Safety Assessments: Vital Signs

Assessments of vital signs will include temperature, pulse rate, respiratory rate, and blood pressure.

Blood pressure and pulse measurements will be assessed in a sitting or supine position with a completely automated device. Manual techniques will be used only if an automated device is not available.

Blood pressure and pulse measurements should be preceded by at least 5 minutes of rest for the participant in a quiet setting without distractions (e.g., television, cell phones).

### Study Procedures and Assessments: Safety Assessments: Physical Examination

A full physical examination will be performed at screening to assess general appearance, skin, eyes, ears, nose, throat neck, cardiovascular, chest and lungs, abdomen, musculoskeletal, neurologic status, mental status and lymphatic systems. For subsequent examinations according to the Schedule of Assessments, physical examinations may be more directed. Each physical examination will include weight; height is only required at screening.

New or worsening clinically significant findings on physical examination will be recorded as AEs if they meet the criteria of an AE.

### Study Procedures and Assessments: Safety Assessments: Eastern Cooperative Oncology Group Performance Status

The ECOG Scale [Oken et al, 1982] will be used to assess performance status (Table 5) at time points outlined in the Schedule of Assessments (FIG. 4).

TABLE 5
Eastern Cooperative Oncology Group (ECOG) Performance Status

| Grade | Description |
|---|---|
| 0 | Fully active, able to carry on all predisease performance without restriction |
| 1 | Restricted in physically strenuous activity, but ambulatory and able to carry out |
| | work of a light or sedentary nature, e.g., light house work, office work |
| 2 | Ambulatory and capable of all self-care, but unable to carry out any work |
| | activities. |
| | Up and about more than 50% of waking hours |
| 3 | Capable of only limited self-care, confined to bed or chair more than 50% of |
| | waking hours |
| 4 | Completely disabled. Cannot carry on any self-care. Totally confined to bed or |
| | chair |
| 5 | Dead |

### Study Procedures and Assessments: Safety Assessments: Electrocardiogram

A standard 12-lead ECG will be performed and assessed as outlined in the Schedule of Assessments (FIG. 4). Any clinically significant abnormal findings at screening should be recorded as medical history and any clinically significant adverse changes on subsequent assessments will be reported as AEs. Additional ECGs or other cardiac assessments (e.g., cardiac enzymes, pulse oximetry) should be performed as clinically indicated or in the event of cardiac symptoms such as chest pain and/or shortness of breath.

Prior to performing ECG, participants should rest in supine position (or semi-recumbent, if supine is not tolerated) for 10 minutes. The 12-lead ECGs will be recorded in triplicate (3 separate ECGs and 1 to 2 minutes apart per time point), with the exception of the single ECG at the EOT visit. All ECGS will be transmitted electronically for central reading.

### Study Procedures and Assessments: Safety Assessments: Order of Assessments

The following order should be followed when more than one assessment is required at a time point:
Vital Signs
ECG
Blood collection

### Study Procedures and Assessments: Adverse Events and Other Safety Aspects

The definitions of an AE or SAE can be found in Appendix 3.

AEs will be reported by the participant (or, when appropriate, by a caregiver, surrogate, or the participant's LAR).

The investigator and any qualified designees are responsible for detecting, documenting and recording events that meet the definition of an AE or SAE and remain responsible for following up AEs that are serious, considered related to the study intervention, or that caused the participant to discontinue the study intervention and/or study (see Appendix 3).

The method of recording, evaluating, and assessing causality of AE and SAE and the procedures for completing and transmitting SAE reports are provided in (Appendix 3).

### Adverse Events and Other Safety Aspects: Time Period and Frequency for Collecting Adverse Event and Serious Adverse Event Information

The Sponsor requires that investigators begin the collection of AEs after the signing of the ICF. In exceptional situations, AE reporting could start at a later time point, depending on the screening requirements. This must be discussed and approved by the PV Product Responsible Person and highlighted at the appropriate governing committee (SRC/PAC). An appropriate period over which AEs are required to be reported after the final administration of study intervention is to be identified taking into account the properties of the study intervention, administration period and disease conditions.

All SAEs will be collected from the signing of the ICF until 30 days after last dose of study intervention at the time points specified in the Schedule of Assessments (FIG. 4) and reported on the eCRF.

All AEs will be collected from the signing of the ICF until 30 days after last dose of study intervention at the time points specified in the Schedule of Assessments (FIG. 4) and reported on the eCRF.

If the severity of an SAE/AE increases, an end date should be provided and the event should be relisted on the eCRF with the new severity and new onset date.

If the severity decreases, an end date should be provided and the SAE/AE should be relisted on the eCRF with the new severity and new onset date. The exception is ongoing pre-dose events that continue postdose and improve postdose. Such events should not be relisted.

If the severity of an SAE reduces, the details of the AE should be provided on the SAE worksheet for the medical assessor to be able to assess the course of the event.

All SAEs will be recorded and reported to the sponsor or designee without undue delay but not later than within 24 hours of obtaining knowledge of the event, as indicated in Appendix 3. The investigator will submit any updated SAE data to the sponsor within 24 hours of it being available.

Investigators are not obligated to actively seek any AE or SAE after conclusion of the study participation. However, if the investigator becomes aware of any SAE with a suspected causal relationship to the study intervention, including a death, at any time after a participant has been discharged from the study, the investigator must promptly report the SAE to the sponsor.

### Adverse Events and Other Safety Aspects: Method of Detecting Adverse Events and Serious Adverse Events

Care will be taken not to introduce bias when detecting AEs and/or SAEs. Open-ended and non-leading verbal questioning of the participant is the preferred method to inquire about AE occurrences.

### Adverse Events and Other Safety Aspects: Follow-Up of Adverse Events and Serious Adverse Events

After the initial AE/SAE report, the investigator is required to proactively follow each participant at subsequent visits/contacts. All SAEs will be followed until resolution, stabilization, the event is otherwise explained, or the participant is lost to follow-up (as defined in below). Further information on follow-up procedures is provided in Appendix 3.

If after the protocol-defined AE collection period described above, an AE progresses to an SAE, or the investigator learns of any (S)AE including death, where the investigator considers there is reasonable possibility it is related to the study intervention or study participation, the investigator must promptly notify the sponsor.

### Adverse Events and Other Safety Aspects: Regulatory Reporting Requirements for Serious Adverse Events

Prompt notification by the investigator to the sponsor of an SAE is essential so that legal obligations and ethical responsibilities towards the safety of participants and the safety of a study intervention under clinical investigation are met.

The sponsor has a legal responsibility to notify both the local regulatory authority and other regulatory agencies about the safety of a study intervention under clinical investigation. The sponsor will comply with country-specific regulatory requirements relating to safety reporting to the regulatory authority, IRB/IEC and investigators.

Investigator safety reports must be prepared for SUSARs according to local regulatory requirements and sponsor policy and forwarded to investigators as necessary.

An investigator who receives an investigator safety report describing an SAE or other specific safety information (e.g., summary or listing of SAEs) from the sponsor will review and then file it along with the IB and will notify the IRB/IEC, if appropriate according to local requirements.

Adverse Events and Other Safety Aspects: Disease-Related Events and/or Disease-Related Outcomes not Qualifying as Adverse Events or Serious Adverse Events

Under this protocol, the following event(s) will not be considered as an (S)AE and should not be reported:
Disease progression: events including defined study endpoints that are clearly consistent with the expected pattern of progression of the underlying disease are not to be recorded as (S)AEs. These data will be captured as efficacy assessment data as outlined in Efficacy Assessments, above. If there is any uncertainty as to whether an event is due to anticipated disease progression and/or if there is evidence suggesting a causal relationship between the IP and the event, it should be reported as an (S)AE. All deaths up to 30 days after the final administration of IP must be reported as an SAE, even if attributed to disease progression.

### Adverse Events and Other Safety Aspects: Special Situations

Certain special situations observed in association with the study intervention, such as incorrect administration (e.g., wrong dose of study intervention) are reported as protocol deviations and/or may require special reporting, as described below. These special situations are not considered AEs, but do require to be communicated to the sponsor as per the timelines defined below.

If a special situation is associated with, or results in, an AE, the AE is to be assessed separately from the special situation and captured as an AE in the eCRF. If the AE meets the definition of an SAE, the SAE is to be reported as described in Appendix 3 and the details of the associated special situation are to be included in the clinical description on the special situation worksheet or pregnancy reporting form.

The special situations are:
Pregnancy
Drug exposure via breast milk
Medication error, overdose and use outside protocol
Misuse/abuse
Occupational exposure
Suspected drug-drug interaction

Instructions and procedures for reporting special situations are provided in Appendix 3.

### Study Procedures and Assessments: Pharmacokinetics

Blood samples for the analysis of Compound I in plasma will be collected as indicated in the Sample Collection Schedule (FIG. 5). The actual date and time of each blood sample collection will be documented.

### Study Procedures and Assessments: Pharmacodynamics

The whole blood samples will be collected from the treated patients to evaluate pharmacodynamics effects of the Compound I. The preclinical in vitro studies have identified CD69 expression by peripheral blood mononuclear cells (PBMCs) as the best pharmacodynamic biomarker candidate. The CD69 is early activation biomarker expressed by many immune cells including T-cells. The blockage of DGKζ by Compound I treatment induces increased expression of CD69 after the TCR mediated stimulation. To evaluate the pharmacodynamics effect of Compound I, the PBMCs will be isolated and stimulated in vitro by CD3/CD28; the expression of the CD69 will be measured by flow cytometry.

Details on sample collection, labeling, storage and shipment procedures will be provided in a separate laboratory manual.

The alternative option for monitoring the pharmacodynamic effects of Compound I may include the direct monitoring of DGKζ enzymatic activity in PBMC, or changes in PBMC gene expression pattern induced by Compound I treatment. Protein extract and RNA isolate may be prepared from PBMC for these assays.

The pharmacodynamic activity of the Compound I will also be estimated using tumor biopsy samples. The DGKζ expression level will be measured in CD8 cells in TME using DGKζ/CD8 immunofluorescence assay. Tumor biopsy samples will be collected pre-dose in cycle 1 (screening/baseline, up to 28 days prior to day 1) and cycle 2 day 1 (on-treatment). The on-treatment tumor biopsy may be performed up to 5 days prior to cycle 2 day 1. If clinically practical, 3 to 4 biopsy cores will be collected at each time point. If at least 3 core samples cannot be provided, the sponsor should be contacted for further guidance. The first biopsy core will be processed to formalin-fixed paraffin-embedded, and the second core stored as frozen tissue. The same priority should be given for 3 and 4 core samples.

Refer to Tumor Biopsy Sample Analysis section, below, for requirements for tumor biopsy collection.

### Study Procedures and Assessments: Pharmacogenomics

Pharmacogenomic (PGx) research may be conducted in the future to analyze or determine genes of relevance to clinical response, pharmacokinetics, and toxicity/safety issues. For PGx assessment, a 4 to 6 mL sample of whole blood will be collected. Samples will be shipped to a sponsor designated banking contract research organization (CRO). Details on the potential PGx analysis cannot be established yet. The sponsor may initiate the PGx analysis in case evidence suggests that genetic variants may be influencing the drug's pharmacokinetics, efficacy and/or safety. Details on sample collection, labeling, storage and shipment procedures will be provided in a separate laboratory manual. See Appendix 6: Pharmacogenomic Analysis with Banked Sample for further details on the banking procedures.

### Study Procedures and Assessments: Biomarkers

The tumor tissue and blood/serum/plasma samples described in the Sample Collection Schedule (FIG. 5) may be used for research purposes to identify genomic/transcriptomics and/or proteomics biomarkers that may be associated with clinical outcome or dynamic changes associated with Compound I treatment, and to confirm mechanism of action associated with modulation of immune response. The details of the planned exploratory biomarker study are below.

### Study Procedures and Assessments: Biomarkers: Peripheral Blood Samples Including Whole Blood and Plasma Sample for Biomarker Analysis

Blood samples will be collected according to the Sample Collection Schedule (FIG. 5). Whole blood samples will be used for immune cells profiling focusing on T- and B-cell subpopulations. The multicolored flow cytometry analysis will be conducted to monitor the changes in patient blood. Additional whole blood sample will be collected for PBMCs isolation. Direct DGK enzymatic activity and its expected suppression by Compound I may be tested in the isolated PBMC samples. The second possible study involving the PBMC cells includes evaluation of the changes in gene expression pattern induced by Compound I, which is to be tested by RNA-seq approach or similar technology.

Separate plasma samples will be collected from patients for evaluation of peripheral cytokine levels. Multiplex Luminex based bead assay measuring more than 50 cytokines and chemokine factors will be used for evaluation of the expected immune system function modulation induced by Compound I treatment.

Finally, pre-treatment and end-treatment plasma samples will be collected for evaluation of potential cell free tumor DNA in patient blood (exploratory plasma biomarker). The analysis may be done retrospectively by using one the panel for sequencing of cell free tumor DNA.

All proposed biomarker analyses dependent on the quality and availability of sufficient materials.

### Study Procedures and Assessments: Biomarkers: Tumor Biopsy Sample Analysis

The investigator, in consultation with other specialists, as needed (e.g., radiology staff) will assess the risk associated with obtaining a tumor tissue sample and determine if the participant is an appropriate candidate for the procedure. Biopsies should be obtained in accordance with institutional policies/guidelines to minimize risk. Ensure that the participant meets all other study eligibility criteria prior to performing the biopsy (as applicable). If clinically practical, participants will undergo tumor biopsy with 3 to 4 core samples collected at each scheduled biopsy time point. If at least 3 core samples cannot be provided, the sponsor should be contacted for further guidance. The primary tumor lesion should be prioritized for biopsy, but samples from metastatic lesions are acceptable. It is highly recommended to have Baseline and On treatment biopsy samples taken from same lesion to eliminate a variability between different lesions. Additional details regarding collection procedures will be provided in the laboratory manual. The tumor biopsy samples may be analyzed for pharmacodynamic and exploratory biomarkers as described in the Pharmacodynamics section, above, and discussed below.

The secondary endpoint objective includes analysis and quantification of the CD4 and CD8 levels in TME, which will be performed with validated immunohistochemistry assay on tumor biopsy formalin-fixed paraffin-embedded sections. Additionally, the changes in activation and proliferative index of CD8 and CD4 cells in TME will be evaluated by measuring Ki-67 expression on these cells. Increased Ki67-index in intra-tumoral CD4/CD8 would be a biomarker of anti-tumor immune response activation and confirmation of Compound I mechanism of action (MOA).

The exploratory evaluation of tumor biopsy may include evaluation of expression of programmed death-ligand 1 (PD-L1) and DGK in TME. One of the commercial PD-L1 assay will be used for evaluation of PD-L1 expression and a custom build assay will be used for evaluation of DGK expression. The biomarkers are considered as a candidate for predictive biomarkers that may be used for identification of the patients who might benefit the most from the Compound I treatment. The predictive power of the biomarkers is to be evaluated in this study.

Exploratory evaluation of tumor biopsy samples may include analysis of TILs and level of their activation. The quantification of CD4, CD8 and FoxP3 positive cells may be monitored in pre- and post-treatment biopsy samples to evaluate the modulation of immune response in TME. Ki67 and PD-1 expression by TILs may be measured to evaluate the level of T cells activation after the treatment.

Additionally, a few multiplex exploratory immunofluorescence and immunohistochemistry-based assays may be conducted to evaluate the level of T cells exhaustion in TME and its potential modulation by the treatment.

Finally, molecular biological profiling of tumor biopsy samples may be conducted to evaluate potential predictive biomarkers, confirm drug MOA, and identify mechanisms of resistance to the Compound I treatment. The molecular biological exploration of tumor biopsy samples may include evaluation of Compound I treatment-induced changes in gene expression in tumor samples. This evaluation may be performed by RNA-seq technology. Tumor mutation burden and tumor mutation profile may be measured using Foundation One panel or similar in the tumor biopsy samples.

### Study Procedures and Assessments: Total Amount of Blood

The total amount of blood for each participant will vary depending on the course of their disease, duration on treatment and local laboratory requirements. At any time during the study, additional blood may be drawn for safety monitoring.

The maximum amount of blood collected within 24 hours is approximately 45 mL on C1D1.

### Participant Discontinuation

Refer to Appendix 1 regarding discontinuation of study sites or of the study as a whole.

Participant Discontinuation: Discontinuation of Individual Participant(s) from Study Intervention

A discontinuation from study intervention is defined as a participant who enrolled in the study and for whom investigational study intervention is permanently discontinued for any reason.

The participant is free to withdraw from the study intervention and/or study for any reason and at any time without giving reason for doing so and without penalty or prejudice. The investigator is also free to discontinue the participant from study intervention or to terminate a participant's involvement in the study at any time if the participant's clinical condition warrants it.

The reason for discontinuation from study intervention must be documented in the participant's medical records.

### Treatment Discontinuation Criteria:

A participant must discontinue study treatment for any of the following reasons:
Participant requests to stop treatment.
Any clinical AE, laboratory abnormality or intercurrent illness, in the opinion of the investigator, indicates continued treatment is not in the best interest of the participant.
Clinical AEs may include the following:
   Participants who have treatment interruption will be permanently discontinued if the investigator or sponsor cannot clearly attribute dose interruption toxicities (detailed above) to a cause other than Compound I and the toxicity does not recover to grade 0 or 1 within 4 weeks.
   Grade ≥2 encephalopathy, meningitis, or motor or sensory neuropathy
   Recurrent toxicities:
   Recurrent grade 2 pneumonitis
   Recurrent grade 3 diarrhea/colitis
   Grade ≥3 pneumonitis
   Any ≥grade 4 toxicities will be permanently discontinued.
   Participants experiencing ≥grade 4 CRS or recurrent grade ≥3 CRS will be discontinued.
Disease progression, as defined by the following:
   Confirmed disease progression by iRECIST (iCPD)
   Disease progression by RECIST 1.1 (i.e., unconfirmed progression by iRECIST, denoted “iUPD”) and the participant is not clinically stable to await subsequent confirmatory scan.
   Clinical disease progression per investigator's assessment
If in the opinion of the investigator a change or discontinuation of therapy would be in the best interest of the participant
Participant is lost to follow-up
Participant begins another anti-cancer therapy
Participant becomes pregnant
Participant remains non-compliant with the protocol based on the investigator or medical monitor
Death
   Participant Discontinuation: Discontinuation of Individual Participant(s) from Study

All participants who discontinue study treatment will remain in the study and must continue to be followed for protocol-specific safety follow-up procedures 30 days after their last dose of Compound I as outlined in FIG. 4. The only exception to this is when the participant specifically withdraws consent for any further contact with him/her or persons previously authorized by the participant to provide this information. If the participant has started a new anti-cancer therapy, he/she will be followed for survival.

For participants who discontinue all study treatment prior to iCPD, the participant will enter the post-treatment follow-up period and continue to undergo imaging assessments every 9 weeks until iCPD or the participant starts another anticancer treatment, whichever occurs first.

All participants who discontinue study treatment are to be followed up for survival according to institutional guidelines, but not less than every 12 weeks after the final safety follow-up visit as outlined in FIG. 4 until death, withdrawal of consent or study closure, whichever occurs first.

### Participant Discontinuation: Lost to Follow-Up

Every reasonable effort is to be made to contact any participant lost to follow-up during the course of the study in order to complete study-related assessments, record outstanding data and retrieve study intervention. These contact attempts should be documented in the participant's medical record.

### Statistical Considerations

### Statistical Considerations: Analysis Sets

The number and percentage of participants will be characterized by each population.

The following populations are defined:

| Population | Description |
|---|---|
| Full Analysis Set (FAS) | All participants who are randomized and receive at least 1 |
| | administration of study intervention and have at least 1 |
| | post-baseline efficacy measurement. |
| Safety Analysis Set | All participants who receive at least 1 administration of study |
| (SAF) | intervention. |
| Pharmacokinetic Analysis | All participants who receive at least 1 administration of study |
| Set (PKAS) | intervention for which concentration data are available to |
| | facilitate derivation of at least 1 primary pharmacokinetic |
| | parameter. Inclusion of participants in the PKAS with missing |
| | data or important protocol deviations will be considered by |
| | the pharmacokineticist on a case-by-case basis. |
| Pharmacodynamic | All participants who receive at least 1 administration of study |
| Analysis Set (PDAS) | intervention for which sufficient pharmacodynamic |
| | measurements were collected. Inclusion of participants in the |
| | PDAS with missing data or important protocol deviations will |
| | be considered on a case-by-case basis. |

### Statistical Considerations: Statistical Analyses

A Statistical Analysis Plan (SAP) will be written to provide details of the analyses, along with specifications for tables, listings and figures to be produced. Changes from the planned analyses in the final SAP that impact the statistical analyses will be justified in the CSR.

### Statistical Considerations: Statistical Analyses: General Considerations

In general, data will be summarized with descriptive statistics for continuous endpoints, and frequency and percentage for categorical endpoints, unless otherwise specified. Percentages by categories will be based on the number of participants with no missing data (i.e., will add up to 100%).

Baseline will be defined as the last non-missing observation prior to first administration of study intervention, unless otherwise specified.

### Statistical Considerations: Statistical Analyses: Primary Endpoint Analysis

The primary analysis for this study is the safety analysis. The safety analysis will consist of data summaries of AEs, DLTs and other safety parameters on the safety analysis set described below.

### Statistical Considerations: Statistical Analyses: Efficacy Analysis

ORR, duration of response (DOR), disease control rate, CR rate, PR rate, progression-free survival (PFS) and OS will be summarized using descriptive statistics. The survival curve and median for time-to-event variables will be estimated using the Kaplan-Meier method and will be reported along with the corresponding 95% confidence interval. The efficacy analysis will be conducted based on the full analysis set (FAS).

### Statistical Considerations: Statistical Analyses: Exploratory Endpoint(s)/Estimand(s) Analysis

### Pharmacodynamic and Predictive Biomarkers

Changes in pharmacodynamic biomarkers will be summarized using descriptive statistics. Correlations with used drug dose and exposure may be evaluated for monotherapy cohorts. The post-treatment changes in biomarker levels will be compared between the monotherapy and combination therapy cohorts. The pretreatment level of the exploratory biomarkers will be evaluated for correlation with response parameters such as ORR, CR/PR rate, PFS and OS. The Kaplan-Meier survival curve for biomarker high and low populations may be built to estimate a biomarker predictivity.

### Radiographic Imaging

A listing of tumor imaging (CT/MRI) data will be provided.

### Statistical Considerations: Statistical Analyses: Safety Analyses Adverse Events

AEs will be coded using MedDRA. An AE with onset at any time from first dosing until last scheduled procedure will be classified as a TEAE for inclusion in the summary tabulations. A drug-related TEAE is defined as any TEAE with a causal relationship assessed as “yes” by the investigator, or records where the relationship is missing.

An overview and separate summaries of the number and percentage of participants with treatment-emergent adverse events (TEAE)s, drug-related TEAEs, TEAEs leading to withdrawal of treatment, drug-related TEAEs leading to withdrawal of treatment and TEAEs excluding SAEs will be presented by SOC, PT and dose level. Also included in the overview are the number and percentage of participants with serious TEAEs, drug-related serious TEAEs, TEAEs leading to death and drug-related TEAEs leading to death.

AE data will be listed.

### Laboratory Assessments

Laboratory parameters will be summarized by cohort and dose level using descriptive statistics shifts in change from baseline, and data listings of clinically significant abnormalities.

### Vital Signs

Vital signs and ECG parameters and their changes from baseline will be summarized by cohort and dose level using descriptive statistics.

### Electrocardiogram

The 12-lead ECG results will be summarized by dose level and time point. A shift analysis table showing shifts from baseline in overall ECG (normal and abnormal) will be provided.

The QT corrected by the QTcF interval will be summarized using frequency tables for each treatment visit for values of clinical importance using the range criteria below.

| | Corrected QT (QTcF) Interval |
|---|---|
| Parameter | Criteria Value (msec) |
| Normal | ≤450 |
| Borderline | >450 |
| Prolonged | >480 |
| Clinically significant | >500 |

The QTc interval will also be summarized by the frequencies of participants with a change from baseline of clinical importance using the criteria identified below. These summaries will be provided for each treatment visit.

| Variable | Change from Baseline |
|---|---|
| Corrected QT (QTcF) Interval (msec) | <0 |
| | ≥0 |
| | >30 |
| | >60 |

Effects of serum concentrations of Compound I on ΔQTcF (defined as the mean change from baseline in QTcF) will be assessed.

Additional details are provided in the SAP.

### Eastern Cooperative Oncology Group Performance Status

Summary statistics (number and percent of participants) for each category of the ECOG performance status at each assessment will be provided. The change from baseline to final visit or early termination will also be summarized. Negative change scores indicate an improvement. Positive scores indicate a decline in performance.

### Statistical Considerations: Statistical Analyses: Pharmacokinetics/Pharmacodynamics Analysis

### Pharmacokinetics Analysis

Descriptive statistics will include number, mean, standard deviation, coefficient of variation (CV), geometric mean, geometric CV, median, minimum and maximum. For the pharmacokinetic parameters tₘₐₓ and t_{lag}, only n, median, minimum and maximum will be calculated.

### Pharmacokinetic Concentrations

Descriptive statistics will be presented for plasma concentrations of Compound I and serum concentrations of pembrolizumab by treatment group and scheduled sample time. Standard graphics including mean plasma concentration-time profiles (linear and semi-log scale) and overlay (spaghetti) plots will be produced.

### Estimation of Pharmacokinetic Parameters

Noncompartmental analysis will be used for the calculation of plasma pharmacokinetic parameters of Compound I using Phoenix version 6.3 or higher (Certara L.P., 100 Overlook Center, Suite 101, Princeton, NJ 08540, US). The urine parameters may be calculated with either Phoenix or SAS®, version 9.3 or higher.

Plasma pharmacokinetic parameters for Compound I will be listed and summarized using descriptive statistics.

### Statistical Analysis of Pharmacokinetic Parameters

Dose proportionality will be evaluated graphically for Compound I AUC₂₄ (AUC₁₂ for twice daily dosing) and Cₘₐₓ for single dose, and AUCtau and Cₘₐₓ for multiple dose using the following linearization of the power model:

$$l n ( pharmacokinetic parameter ) = {β}_{0} + {β}_{1} \cdot l n ( dose )$$

where β₀ is the intercept and β₁ is the slope. Dose proportionality will be declared if the 90% CI for β₁ lies entirely within the critical region

$$( 1 + \frac{l n ( 0.5 )}{l n ( r )} , 1 + \frac{l n ( 2 )}{l n ( r )} ) ,$$

where r is the ratio of the highest and the lowest dose used in the model.

Natural logarithm-transformed scatter plots (In-ln), including the regression line and a reference line with a slope of 1 will be displayed.

Steady state will be evaluated using a visual inspection of individual participant trough concentrations versus day (spaghetti plot) overlaid with a mean profile.

### Concentration-Response Relationship Analysis

Concentration-response relationship may be explored. Details will be described in exposure-response analysis plan if applicable.

### Pharmacodynamics | Immunogenicity Analysis

Descriptive statistics will be used to summarize changes in secondary and exploratory endpoint biomarkers such as TILs and their proliferative index in TME, plasma levels of serum cytokines, levels of CD69 expression and peripheral T- and B-cell subpopulation, changes in protein expression (PD-L1, DGKζ) in TME. ADA against pembrolizumab will be listed and summarized. The summary will be presented by treatment cohort.

The correlation between the investigational predictive biomarkers such as tumor mutation profile, tumor mutation burden, PD-L1, DGK, TIL levels in tumor samples, and clinical response parameters (e.g., ORR, DOR, OS and PFS) will be evaluated.

### Statistical Considerations: Statistical Analyses: Other Analyses

### Analysis of Exploratory Biomarker(s)

Associations between biomarkers and clinical results (efficacy, safety or pharmacodynamics) may be performed on participants who have the necessary baseline and on-study measurements to provide interpretable results for specific parameters of interest. Biomarkers may be summarized graphically or descriptively as they relate to clinical measures, as applicable. Summary statistics may be tabulated. Additional post-hoc analyses, such as alternative modeling approaches, may be conducted. All analyses described in this section are based on availability of the data.

Details will be described in a Supplementary Biomarker Analysis Plan.

### Statistical Considerations: Interim Analysis

The sponsor will perform an interim analysis for efficacy of each tumor-specific expansion cohort will be performed when 20 participants are enrolled with evaluable tumor response data in that cohort.

### Statistical Considerations: Sample Size Determination

Approximately 196 participants are planned for enrollment in this study; however, the total sample size will vary based on observed safety and efficacy responses for each cohort.

Part 1: Dose Escalation: Up to 36 participants will be enrolled in dose escalation. Participants that are not considered evaluable for the DLT period may be replaced. The sample size in the dose escalation cohorts is based on TITE-BOIN dose escalation design and not based on power calculation.

Part 2: Dose Expansion: Approximately 160 participants will be enrolled in the dose expansion phase. Initially, 20 participants will be enrolled into each tumor expansion cohort. Based on responses observed in an expansion cohort, up to 20 additional participants may be enrolled in that tumor specific expansion cohort. The Bayesian optimal phase 2 (BOP2) design [Zhou et al., 2017] will be used in the study to evaluate the efficacy in terms of ORR rate for the selected dose level. With assumption of the efficacious ORR being 10% and the inefficacious ORR being 2%, the statistical power would be approximately 73.8% while controlling the type I error rate at 4.1%.

### APPENDICES

### Appendix 1: Ethical, Regulatory and Study Oversight Considerations

### Regulatory and Ethical Considerations

This study will be conducted in accordance with the protocol and with the following:
Consensus ethical principles derived from international guidelines including the Declaration of Helsinki and CIOMS international ethical guidelines
Applicable ICH GCP guidelines
Applicable laws and regulations

The protocol, protocol amendments, ICF, IB, and other relevant documents (e.g., advertisements) must be submitted to an IRB/IEC by the investigator and reviewed and approved by the IRB/IEC before the study is initiated.

Any amendments to the protocol will require IRB/IEC approval before implementation of changes made to the study design, except for changes necessary to eliminate an immediate hazard to study participants.

The investigator will be responsible for the following:
Providing written summaries of the status of the study to the IRB/IEC annually or more frequently in accordance with the requirements, policies, and procedures established by the IRB/IEC
Notifying the IRB/IEC of SAEs or other significant safety findings as required by IRB/IEC procedures
Providing oversight of the conduct of the study at the site and adherence to requirements of 21 CFR, ICH guidelines, the IRB/IEC, European regulation 536/2014 for clinical studies (if applicable), and all other applicable local regulations

### Informed Consent of Participants: Informed Consent Process

The investigator or the investigator's representative will explain the nature of the study to the potential participant or their legally authorized representative and answer all questions regarding the study.

The information provided shall be provided in writing and shall:
Enable the participant or their legally authorized representative to understand:
   The nature, objectives, benefits, implications, risks and inconveniences of the clinical study;
   The conditions under which the clinical study is to be conducted, including the expected duration of the participant's participation in the clinical study;
   The possible treatment alternatives, including the follow-up measures if the participation of the participant in the clinical study is discontinued;
Be kept comprehensive, concise, clear, relevant, and understandable to a layperson;
Be provided in a prior interview with an appropriately qualified member of the study team. Special attention shall be paid to the information needs of specific patient populations and of individual participants, as well as to the methods used to give the information. Care should be taken to verify that the participant has understood the information;
Include information about the applicable damage compensation system;
Include the study ISN number and information about the future availability of the clinical study results in terms understandable to a layperson;
Potential participants must be informed that their participation is voluntary and shall have their protective rights and guarantees explained. In particular their right to refuse to participate and the right to withdraw from the clinical study at any time without any resulting detriment and without having to provide any justification shall be explained.

Participants or their legally authorized representative defined as an individual, judicial or other body authorized under applicable law to consent on behalf of a prospective patient to the patient's participation in the procedure(s) involved in research (45 CFR 46.102[c]), will be required to sign a statement of informed consent that meets the requirements of 21 CFR 50, local regulations, ICH guidelines, HIPAA requirements, where applicable, and the IRB/IEC or study center.

If the participant is a minor who is capable of forming an opinion and assessing the information given, their assent, in order to participate in a clinical study, shall also be obtained.

The medical record must include a statement that written informed consent was obtained before the participant was enrolled in the study and the date the written consent was obtained. The authorized person obtaining the informed consent must also sign the ICF.

Participants must be reconsented to the most current version of the ICF(s) during their participation in the study.

A copy of the ICF(s) must be provided to the participant or their legally authorized representative.

### Informed Consent of Participants: Supply of New and Important Information Influencing the Participant's Consent and Revision of the Written Information

The investigator or the investigator's representative will immediately inform the participant verbally whenever new information becomes available that may be relevant to the participant's consent or may influence the participant's willingness to continue participating in the study (e.g., report of serious adverse drug reaction). The communication must be documented in the participant's medical records and whether the participant is willing to remain in the study or not must be confirmed and documented.

The investigator must update the participant's ICF and submit it for approval to the IRB/IEC. The investigator or the investigator's representative must obtain written informed consent from the participant on all updated ICFs throughout their participation in the study. The investigator or the investigator's designee must reconsent participants with the updated ICF even if relevant information was provided verbally. The investigator or the investigator's representative who obtained the written informed consent and the participant should sign and date the ICF. A copy of the signed ICF will be given to the participant and the original will be placed in the participant's medical record. An entry must be made in the participant's records documenting the reconsent process.

### Data Protection

The sponsor will use the personal data collected from participants in order to run the study and to use and publish the results of the study. The personal data of participants will be used throughout the development program of the investigational study intervention; e.g., to develop a product, obtain permission to market the product, monitor its safety and obtain coverage by health insurance and reimbursement schemes.

The sponsor relies on the permission (or “consent”) in order to use the data of participants and their permission is obtained by signing the ICF.

The investigator and/or the site personnel will record information from the medical file of study participants in eCRF and in an external (electronic) data file (e.g., central laboratory data). These study records will identify the participants with a code instead of their name or other personal data. Only the investigator and the site personnel can match the code with the name of the study participant which will be retained in the medical file at the site only. Non-medical personnel acting on behalf of the sponsor and being bound by a duty of confidentiality, as well as health authorities and/or IRB/IECs, may also be given access to this data at the site only to verify that the study is carried out in compliance with legal and quality requirements.

The sponsor collects personal data from the participants during the study that may be used for:
Submission to government regulatory authorities and IRB/IEC
Use in reports or public scientific presentations, and
Use in research, now or in the future.

However, the identity of participants will not be revealed if study participants' personal data are shared for these purposes.

The sponsor will inform the participants about their privacy rights and how to exercise them under the ICF which participants will sign in order to participate in the study. The sponsor will provide the investigator and study staff with a privacy notice explaining how their personal data will be used and how to exercise their privacy rights.

The sponsor will comply and process personal data in accordance with all applicable privacy laws and regulations.

The contract between sponsor and study sites specifies responsibilities of the parties related to data protection, including handling of data security breaches and respective communication and cooperation of the parties.

Information technology systems used to collect, process, and store study-related data are secured by technical and organizational security measures designed to protect such data against accidental or unlawful loss, alteration, or unauthorized disclosure or access.

### Committee(s) Structure

A DESC consisting of sponsor representatives and investigators will convene once a dose level cohort completes the DLT observation period and the data are available for review. The DESC's decision on the dose level for the next cohort will be guided by the TITE-BOIN, which is based on DLTs observed at each dose level.

While safety data from the DLT observation period in the escalation cohorts are the minimum safety data needed for the committee meeting, all available safety findings, including those occurring after the designated DLT observation period that meet DLT criteria (“delayed DLT”), will be considered by the DESC. The DESC will assess whether a longer DLT observation period is warranted, based on emerging data. Additionally, the DESC may choose a more conservative dosing decision than indicated by the TITE-BOIN, based on evaluation of the safety data and available pharmacokinetic data.

The DESC will also review the aggregate safety data from the expansion cohorts. Based on the available data, the DESC may choose more conservative stopping rules for the expansion cohorts than those outlined in the Sample Size Justification section below.

Following the DESC review, the sponsor will determine the final RP2D based on the efficacy and safety in the dose optimization expansion cohort.

### Dissemination of Clinical Study Data

ICH E3 guidelines recommend and EU Clinical Trial Regulation 536/2014 requires that a final CSR that forms part of a marketing authorization application, be signed by the representative for the coordinating investigator(s) or the principal investigator(s). The representative for the coordinating investigator(s) or the principal investigator(s) will have the responsibility to review the final study results to confirm to the best of their knowledge it accurately describes the conduct and results of the study. The representative for the coordinating investigator(s) or the principal investigator(s) will be selected from the participating investigators by the sponsor prior to database lock.

### Data Quality Assurance

All participant data relating to the study will be recorded on eCRF unless transmitted to the sponsor or designee electronically in an external data file (e.g., central laboratory data). The investigator is responsible for verifying that data entries on the eCRF are accurate and correct by physically or electronically signing the eCRF.

Guidance on completion of CRFs will be provided in a separate eCRF Completion Guideline.

The investigator must permit study-related monitoring, audits, IRB/IEC review, and regulatory agency inspections and provide direct access to source data documents.

QTLs will be predefined in the applicable plan(s) to identify systematic issues that can impact participant safety and/or reliability of study results. These predefined parameters will be monitored during the study, and important deviations from the QTLs and remedial actions taken will be summarized in the CSR.

Monitoring details describing strategy, including definition of study critical data items and processes (e.g., risk-based initiatives in operations and quality such as risk management and mitigation strategies and analytical risk-based monitoring), methods, responsibilities and requirements, including handling of noncompliance issues and monitoring techniques (central, remote or on-site monitoring) are provided in the Monitoring Plan.

The sponsor or designee is responsible for the data management of this study including quality checking of the data.

The sponsor assumes accountability for actions delegated to other individuals (e.g., CROs).

Records and documents, including signed ICFs, pertaining to the conduct of this study must be retained by the investigator according to ICH or applicable local regulatory requirements, whichever is longer, after study completion. No records may be destroyed during the retention period without the written approval of the sponsor. No records may be transferred to another location or party without written notification to the sponsor.

### Study and Site Start and Closure

### First Act of Recruitment

The study start date is the date on which the clinical study will be open for recruitment of participants.

The first act of recruitment is the date the first participant signs the ICF.

### Study/Site Termination

The sponsor or designee reserves the right to close the study site or terminate the study at any time for any reason at the sole discretion of the sponsor. Study sites will be closed upon study completion. A study site is considered closed when all required documents and study supplies have been collected and a study-site closure visit has been performed.

The investigator may initiate study-site closure at any time, provided there is reasonable cause and sufficient notice is given in advance of the intended termination.

Reasons for the early closure of a study site by the sponsor or investigator may include but are not limited to:

For study termination:
Discontinuation of further investigational study intervention development

For site termination:
Failure of the investigator to comply with the protocol, the requirements of the IRB/IEC or local health authorities, the sponsor's procedures or GCP guidelines
Inadequate or no recruitment (evaluated after a reasonable amount of time) of participants by the investigator
Total number of participants enrolled earlier than expected

If the study is prematurely terminated or suspended, the sponsor or designee shall promptly inform the investigators, the IECs/IRBs, the regulatory authorities, and any CRO(s) used in the study of the reason for termination or suspension, as specified by the applicable regulatory requirements. The investigator shall promptly inform the participant and should assure appropriate participant therapy and/or follow-up.

### Quality Assurance

The sponsor is implementing and maintaining QA and QC systems with written SOPs to ensure that studies are conducted and data are generated, documented, recorded, and reported in compliance with the protocol, GCP and applicable regulatory requirement(s). Where applicable, the QA and QC systems and written SOPs of the CRO will be applied.

The sponsor or sponsor's designee may arrange to audit the study at any or all study sites and facilities. The audit may include on-site review of regulatory documents, CRFs and source documents. Direct access to these documents will be required by the auditors.

### Appendix 2: Contraception Requirements

Woman of childbearing potential who are eligible for participation in the study, including those who choose complete abstinence, must have pregnancy tests as specified in the Schedule of Assessments (FIG. 4). Pregnancy test results must confirm that the participant is not pregnant.

### Women of Childbearing Potential Definitions and Methods of Contraception Definitions

A female is considered fertile (i.e., woman of childbearing potential) following menarche and until becoming postmenopausal unless permanently sterile.

### Females in the Following Categories are not Considered Woman of Childbearing Potential

Premenarchal
Premenopausal with 1 of the following (i.e., permanently sterile):
   Documented hysterectomy
   Documented bilateral salpingectomy
   Documented bilateral oophorectomy
Postmenopausal

A postmenopausal state is defined as at least 12 months after last menstrual bleeding without an alternative medical cause.

In case the last menstrual bleeding cannot be clearly determined, confirmation with more than 1 FSH measurement of at least >40 IU/L (or higher per local institutional guidelines) is required.

Females on HRT and whose menopausal status is in doubt will be required to use 1 of the nonestrogen hormonal highly effective contraception methods if they wish to continue their HRT during the study. Otherwise, they must discontinue HRT to allow confirmation of postmenopausal status by repeated FSH measurements before study enrollment.

Documentation of any of these categories can come from the study site personnel's review of the female participant's medical records, medical examination or medical history interview.

### Contraception Guidance for Female Participants of Childbearing Potential

Female participants of childbearing potential are eligible for participation in the study if they agree to use one of the highly effective methods of contraception listed below from the time of signing the ICF and until the end of relevant systemic exposure, defined as 30 days after the final study intervention administration.^{a}

Highly effective methods of contraception (failure rate of <1% per year when used consistently and correctly)^{b}:
Combined (estrogen- and progestogen-containing) hormonal contraception associated with inhibition of ovulation
   Oral
   Intravaginal
   Transdermal
Progestogen-only hormonal contraception associated with inhibition of ovulation
   Oral
   Injectable
   Implantable
Other combined (estrogen- and progesterone-containing) methods
   Vaginal ring
   Injectable
   Implantable
   Intrauterine hormone-releasing system or intrauterine device
Bilateral tubal occlusion or bilateral tubal ligation
Vasectomized partner: A vasectomized partner is a highly effective contraception method provided that the partner is the sole male sexual partner of the woman of childbearing potential and the absence of sperm has been confirmed. If not, an additional highly effective method of contraception should be used.
^{a} Local laws and regulations may require use of alternative and/or additional contraception methods.
^{b} Typical use failure rates may differ from those when used consistently and correctly. Use should be consistent with local regulations regarding the use of contraceptive methods for participants participating in clinical studies.
   Contraception Guidance for Male Participants with Partner(s) of Childbearing Potential.

Male participants with female partners of childbearing potential are eligible for participation in the study if they agree to the following during treatment and until the end of relevant systemic exposure defined as 30 days after final study intervention administration.^{a}
Inform any and all partner(s) of their participation in a clinical study and the need to comply with contraception instructions as directed by the investigator
Use a condom

Female partners of male participants who have not undergone a vasectomy with the absence of sperm confirmed or a bilateral orchiectomy should consider use of effective methods of contraception
^{a} Local laws and regulations may require use of alternative and/or additional contraception methods.

### Contraception Requirements: Highly Effective Birth Control Methods-Failure Rate <1%/Year

1. Combined estrogen- and progesterone-containing hormonal contraception
   a. Oral
   b. Intravaginal
   c. Transdermal e.g. Patch
   d. Injectable e.g., Cyclofem, Mesigyna
2. Progestogen-only hormonal contraception
   a. Oral
   b. Injectable e.g., DMPA-IM or -SC
   c. Implantable e.g., Norplant
3. IUD
4. IUS
5. Bilateral tubal occlusion
6. Vasectomized male partner
7. True abstinence: (When this is in line with the preferred and usual lifestyle of the participant. Periodic abstinence (such as calendar, ovulation, symptothermal, post-ovulation methods) and withdrawal are not acceptable methods of contraception. Sexual abstinence is considered a highly effective method only if defined as refraining from heterosexual intercourse during the entire period of risk associated with the study intervention. It is not necessary to use any other method of contraception when complete abstinence is elected.

### Birth Control Methods Considered Unacceptable:

1. Periodic abstinence (calendar, ovulation, symptothermal, post-ovulation methods)
2. Withdrawal (coitus interruptus)
3. Spermicides only
4. Lactational amenorrhea

### Appendix 3: Adverse Events: Definitions and Procedures for Recording, Evaluating, Follow-Up and Reporting

### Definition of Adverse Events (AEs)

### AE Definition:

An AE is any untoward medical occurrence in a patient or clinical study participant, temporally associated with the use of study intervention, whether or not considered related to the study intervention.

NOTE: An AE can therefore be any unfavorable and unintended sign (including an abnormal laboratory finding), symptom, or disease (new or exacerbated) temporally associated with the use of study intervention. This includes events related to the comparator and events related to the (study) procedures.

### Events Meeting the AE Definition

Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the investigator (i.e., not related to progression of underlying disease).

Exacerbation of a chronic or intermittent pre-existing condition including either an increase in frequency and/or intensity of the condition.

New conditions detected or diagnosed after study intervention administration even though it may have been present before the start of the study.

### Events not Meeting the AE Definition

Any clinically significant abnormal laboratory findings or other abnormal safety assessments which are associated with the underlying disease, unless judged by the investigator to be more severe than expected for the participant's condition.

The disease/disorder being studied or expected progression, signs, or symptoms of the disease/disorder being studied, unless more severe than expected for the participant's condition.

Medical or surgical procedure (e.g., endoscopy, appendectomy): the condition that leads to the procedure is the AE.

Situations in which an untoward medical occurrence did not occur (social and/or convenience admission to a hospital).

Anticipated day-to-day fluctuations of pre-existing disease(s) or condition(s) present or detected at the start of the study that do not worsen.

### Definition of Adverse Events: Abnormal Laboratory Findings

Any abnormal laboratory test result e.g., hematology, biochemistry or urinalysis or other safety assessment e.g., vital signs, physical examination, ECGs or radiographic scans, including those that worsen from baseline, that is considered to be clinically significant in the medical and scientific judgment of the investigator and not related to underlying disease, is to be reported as an (S)AE.

Any clinically significant abnormal laboratory finding or other abnormal safety assessment, which is associated with the underlying disease, does not require reporting as an (S)AE, unless judged by the investigator to be more severe than expected for the participant's condition.

Repeating an abnormal laboratory test or other safety assessment, in the absence of any of the above criteria, does not constitute an AE. Any abnormal test result that is determined to be an error does not require reporting as an AE.

### Definition of Adverse Events: Potential Cases of Drug-Induced Liver Injury

Refer to Appendix 4 for detailed instructions on drug induced liver injury. Abnormal values in AST and/or ALT concurrent or with abnormal elevations in TBL that meet the criteria outlined in Appendix 4, in the absence of other causes of liver injury, are considered potential cases of drug-induced liver injury (potential Hy's Law cases) and are always to be considered important medical events and reported per reporting procedures for SAEs.

### Definition of Serious Adverse Events (SAEs)

An SAE is defined as any untoward medical occurrence that, at any dose:
Results in death
Is life-threatening

The term “life-threatening” in the definition of “serious” refers to an event in which the participant was at risk of death at the time of the event. It does not refer to an event, which hypothetically might have caused death, if it were more severe.
Requires inpatient hospitalization or prolongation of existing hospitalization
   In general, hospitalization signifies that the participant has been detained (usually involving at least an overnight stay) at the hospital or emergency ward for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious.
   Hospitalization for elective treatment of a pre-existing condition that did not worsen from baseline is not considered an AE.
Results in persistent or significant disability/incapacity
   The term disability means a substantial disruption of a person's ability to conduct normal life functions.
   This definition is not intended to include experiences of relatively minor medical significance such as uncomplicated headache, nausea, vomiting, diarrhea, influenza, and accidental trauma (e.g., sprained ankle), which may interfere with or prevent everyday life functions but do not constitute a substantial disruption.
Is a congenital anomaly/birth defect
Other situations:
   Medical or scientific judgment should be exercised in deciding whether SAE reporting is appropriate in other situations such as important medical events that may not be immediately life-threatening or result in death or hospitalization but may jeopardize the participant or may require medical or surgical intervention to prevent 1 of the other outcomes listed in the above definition. These events should usually be considered serious.
   Examples of such events include invasive or malignant cancers, intensive treatment in an emergency room or at home for allergic bronchospasm, blood dyscrasias or convulsions that do not result in hospitalization, or development of drug dependency or drug abuse.

If an event is not an AE per definition in [Section 0], then it cannot be an SAE even if serious conditions are met (e.g., hospitalization for signs/symptoms of the disease under study, death due to progression of disease).

### Assessment of Causality

The investigator is obligated to assess the relationship between study intervention, study procedures and each occurrence of each AE/SAE.

A “reasonable possibility” of a relationship conveys that there are facts, evidence, and/or arguments to suggest a causal relationship, rather than a relationship cannot be ruled out.

The investigator will use clinical judgment to determine the relationship.

Alternative causes, such as underlying disease(s), concomitant therapy, and other risk factors, as well as the temporal relationship of the event to study intervention administration will be considered and investigated.

The investigator will also consult the IB and/or product information, for marketed products, in the assessment.

For each AE/SAE, the investigator must document in the medical notes that they have reviewed the AE/SAE and has provided an assessment of causality.

There may be situations in which an SAE has occurred and the investigator has minimal information to include in the initial report to the sponsor. However, it is very important that the investigator always make an assessment of causality for every event before the initial transmission of the SAE data to the sponsor.

The investigator may change their opinion of causality in light of follow-up information and send a SAE follow-up report with the updated causality assessment.

The causality assessment between study intervention is 1 of the criteria used when determining regulatory reporting requirements.

Following a review of the relevant data, the causal relationship between the study intervention and each (S)AE will be assessed by answering “yes” or “no” to the question “Do you consider that there is a reasonable possibility that the event may have been caused by the study intervention?”

When making an assessment of causality, the following factors are to be considered when deciding if there is evidence and/or arguments to suggest there is a “reasonable possibility” that an (S)AE may have been caused by the study intervention (rather than a relationship cannot be ruled out) or if there is evidence to reasonably deny a causal relationship:
Has the participant been administered study intervention?
Plausibility (i.e., could the event have been caused by the suspect study intervention?Consider biologic and/or pharmacologic mechanism, half-life, literature evidence, drug class, preclinical and study data, etc.)
Dechallenge/dose reduction/rechallenge:
   Dechallenge: Did the (S)AE resolve or improve after only stopping the dose of the suspect study intervention without any treatment?
   Dose reduction: Did the (S)AE resolve or improve after reducing the dose of the suspect study intervention?
   Rechallenge: Did the (S)AE reoccur if the suspected study intervention was reintroduced after having been stopped?
Laboratory or other test results: a specific laboratory investigation supports the assessment of the relationship between the (S)AE and the study intervention (e.g., based on values pre-, during and post-treatment)
Available alternative explanations independent of study intervention exposure; such as other concomitant interventions, medical history, concurrent or underlying disease, risk factors including medical and family history, season, location, etc., and strength of the alternative explanation
Temporal relationship between exposure to the study intervention and (S)AE onset and/or resolution. Did the (S)AE occur in a reasonable temporal relationship to the administration of the study intervention?
Finally, judging which are more likely based on all the above contents, factors of reasonable possibility or confounding factors, comprehensive judgment of plausible will be provided.

There may be situations in which an SAE has occurred and the investigator has minimal information to include in the initial report to the sponsor. While it is very important that the investigator always assesses causality for every event before the initial transmission of the SAE data to the sponsor, the initial report should be submitted without delay (i.e., within 24 hours of awareness). With limited or insufficient information about the event to make an informed medical judgment and in absence of any indication or evidence to establish a causal relationship, a causality assessment of “no” is to be considered. In such instance, the investigator is expected to obtain additional information regarding the event as soon as possible and to re-evaluate the causality upon receipt of additional information. The medically qualified investigator may revise their assessment of causality in light of new information regarding the SAE and shall send an SAE follow-up report and update the eCRF with the new information and updated causality assessment.

### Assessment of Severity

Adverse events, including abnormal clinical laboratory values, will be graded using the National Cancer Institute-Common Terminology Criteria for Adverse Event (NCI-CTCAE) guidelines, version 5.0. The items that are not stipulated in the NCI-CTCAE version 5.0 will be assessed according to the criteria in Table 6, below and entered into the eCRF:

TABLE 6
Grading Scale Defining the Severity of an Adverse Event

| Grade | Assessment Standard |
|---|---|
| 1 - Mild | Asymptomatic or mild symptoms, clinical or diagnostic observations |
| | only; intervention not indicated |
| 2 - Moderate | Minimal local or noninvasive intervention indicated; limiting age- |
| | appropriate instrumental ADL† |
| 3 - Severe | Medically significant but not immediately life-threatening, |
| | hospitalization or prolonged hospitalization indicated; disabling; |
| | limiting self-care ADL‡ |
| 4 - Life- | Life-threatening consequences, urgent intervention indicated |
| threatening | |
| 5 - Death | Death related to AE |

ADL: activities of daily living;
AE: adverse event
†Instrumental ADL refer to preparing meals, shopping for groceries or clothes, using the telephone, managing money, etc.
‡Self-care ADL refer to bathing, dressing and undressing, feeding self, using the toilet, taking medications and not bedridden.

Recording and Follow-Up of AEs and/or SAEs

### AE and SAE Recording

When an AE/SAE occurs, it is the responsibility of the investigator to review all documentation (e.g., hospital progress notes, laboratory reports, and diagnostics reports) related to the event.

The investigator will then record all relevant AE/SAE information in the eCRF.

It is not acceptable for the investigator to send photocopies of the participant's medical records to the sponsor in lieu of completion of the eCRF.

There may be instances when copies of medical records for certain cases are requested by the sponsor. In this case, all participant identifiers, with the exception of the participant number, will be redacted on the copies of the medical records before submission to the sponsor.

The investigator will attempt to establish a diagnosis of the event based on signs, symptoms, and/or other clinical information. Whenever possible, the diagnosis (not the individual signs/symptoms) will be documented as the AE/SAE.

### Follow-Up of AEs and SAEs

The investigator is obligated to perform or arrange for the conduct of supplemental measurements and/or evaluations as medically indicated or as requested by the sponsor to elucidate the nature and/or causality of the AE or SAE as fully as possible. This may include additional laboratory tests or investigations, histopathological examinations, or consultation with other health care professionals.

If a participant dies during participation in the study or during a recognized follow-up period, the investigator will provide the sponsor with a copy of any postmortem findings including histopathology.

New or updated information will be recorded in the originally completed eCRF.

The investigator will submit any updated SAE data to the sponsor within 24 hours of receipt of the information.

### Reporting Procedures for SAEs

The investigator must complete and submit an SAE worksheet containing all information that is required by local and/or regional regulations to the sponsor by fax or email immediately (no later than within 24 hours of obtaining knowledge of the event).

The SAE worksheet must be signed by a medically qualified investigator (as identified on delegation of authority log). Signature confirms accuracy and completeness of the SAE data, as well as the investigator causality assessment including the explanation for the causality assessment.

If the SAE is associated with emergency unblinding by the investigator as outlined above, this is to be recorded on the SAE worksheet. On the SAE worksheet, the investigator is to include when unblinding took place in association with the SAE.

Follow-up information for the event should be sent promptly (as soon as available, but no longer than within 7 days of the initial notification).

Full details of the SAE should be recorded on the medical records and SAE/special situation worksheet.

The following minimum information is required:
International study number/study number
Participant number, sex and age
Date of report
Description of the SAE (event and seriousness criteria)
Causal relationship to the study intervention (including reason)
Drug provided (if any)

The sponsor or sponsor's designee will medically evaluate the SAE and determine if the report meets the requirements for expedited reporting based on seriousness, causality, and expectedness of the events (e.g., SUSAR reporting) according to current local/regional regulatory requirements. The sponsor or sponsor's designee will submit expedited safety reports to competent authorities and concerned ethics committee per current local regulations, and will inform the investigators of such regulatory reports as required. Investigators must submit safety reports as required by their IRB/IEC within timelines set by regional regulations (e.g., EMA, FDA) where required. Documentation of the submission to and receipt by the IRB/IEC of expedited safety reports should be retained by the study site. In the US, FDA expedited IND reporting guidelines will be followed.

The sponsor will notify all investigators responsible for ongoing clinical studies with the study intervention of all SUSARs, which require submission per local requirements IRB/IEC/head of the study site

The investigators should provide written documentation of IRB/IEC notification for each report to the sponsor.

### Reporting Procedures for Pregnancy and Special Situations: Contraceptive Guidance and Collection of Pregnancy Information

Details of all pregnancies in female participants and, if indicated, female partners of male participants will be collected after the start of study intervention or within 30 days from the discontinuation of dosing

If a pregnancy is reported, the investigator will record pregnancy information on the appropriate form and submit it to the sponsor within 24 hours of learning of the female participant or female partner of male participant (after obtaining the necessary signed informed consent from the female partner) pregnancy.

While pregnancy itself is not considered to be an AE or SAE, any pregnancy complication or elective termination of a pregnancy for medical reasons will be reported as an AE or SAE.

Abnormal pregnancy outcomes (e.g., spontaneous abortion, fetal death, stillbirth, congenital anomalies, ectopic pregnancy) are considered SAEs and will be reported as such.

The participant/pregnant female partner will be followed to determine the outcome of the pregnancy. The investigator will collect follow-up information on the participant/pregnant female partner and the neonate and the information will be forwarded to the sponsor.

Any pregnancy-related SAE reported after the participant's last study visit, considered reasonably related to the study intervention by the investigator, will be reported to the sponsor as described above. While the investigator is not obligated to actively seek this information in former study participants/pregnant female partners, they may learn of pregnancy-related SAE through spontaneous reporting.

Any female participant who becomes pregnant while participating in the study will discontinue study intervention or be withdrawn from the study.

### Reporting Procedures for Pregnancy and Special Situations: Medication Error, Overdose and “Off-label Use”

If a medication error (defined as an unintended failure in the treatment process that leads to, or has the potential to lead to, harm to the participant), overdose or “off-label use” (i.e., use outside of the target disease defined in the protocol) is suspected, the investigator must forward the special situation worksheet to the sponsor by fax or email immediately (within 24 hours of awareness). Any associated (S)AEs are to be reported in the eCRF. If the AE meets the definition of an SAE, the SAE is also to be reported as described above together with the details of the medication error, overdose and/or “off-label use.” Reporting Procedures for Pregnancy and Special Situations: Misuse/Abuse

Definition of misuse: Situations where the study intervention is/are intentionally and inappropriately used not in accordance with the intended use as defined in the protocol.

Definition of abuse: Persistent or sporadic, intentional excessive use of medicinal products which is accompanied by harmful physical or psychological effects.

If misuse or abuse of the study intervention is suspected, the investigator must forward the special situation worksheet to the sponsor by fax or email immediately (within 24 hours of awareness). Any associated (S)AEs are to be reported in the eCRF. If the AE meets the definition of an SAE, the SAE is also to be reported as described above together with details of the misuse or abuse of the study intervention.

### Reporting Procedures for Pregnancy and Special Situations: Occupational Exposure

If occupational exposure (e.g., inadvertent exposure to the study intervention of study site personnel while preparing it for administration to the participant) to the study intervention occurs, the investigator must forward the special situation worksheet to the sponsor by fax or email immediately (within 24 hours of awareness). Any associated (S)AEs occurring to the individual associated with or resulting from the special situation are to be reported on the special situations worksheet.

### Reporting Procedures for Pregnancy and Special Situations: Suspected Drug-Drug Interaction

If a drug-drug interaction associated with the study intervention is suspected, the investigator must forward the special situation worksheet to the sponsor by fax or email immediately (within 24 hours of awareness). Any associated (S)AEs are to be reported in the eCRF. If the AE meets the definition of an SAE, the SAE is also to be reported as described above together with details of the suspected drug-drug interaction.

### Supply of New Information Affecting the Conduct of the Study

When new information becomes available that is necessary to allow for proper conduct of the study, the sponsor will inform all investigators involved in the study, as well as the appropriate regulatory authorities. Investigators should inform the IRB/IEC of such information when needed.

The investigator will also inform the participants, who will be required to sign an updated ICF in order to continue in the study.

### Urgent Safety Measures

An USM is an intervention that is not defined by the protocol and can be put in place with immediate effect without needing to gain prior approval by the sponsor, relevant CA, IRB/IEC, where applicable, in order to protect participants from any immediate hazard to their health and/or safety. Either the investigator or the sponsor can initiate a USM. The cause of a USM can be safety-, product- or procedure-related.

### Reporting Urgent Safety Measures

In the event of a potential USM, the investigator must contact the study physician (within 24 hours of awareness). Full details of the potential USM are to be recorded in the participant's medical records. The sponsor may request additional information related to the event to support their evaluation.

If the event is confirmed to be a USM, the sponsor will take appropriate action to ensure the safety and welfare of the participants. These actions may include, but are not limited to, a change in study procedures or study intervention, halting further enrollment in the study, or stopping the study in its entirety. The sponsor or sponsor's designee will notify the relevant competent authorities and concerned ethics committee within the timelines required per current local regulations, and will inform the investigators, as required. When required, investigators must notify their IRB/IEC within timelines set by regional regulations.

### Appendix 4: Liver Safety Monitoring and Assessment

The purpose of this appendix is to provide guidance for the monitoring of drug-induced liver injury during the course of the study. It should be noted that this section does not specify the end of study analyses of liver enzymes. The end of study liver enzymes analyses will be described in the SAP. Any participant enrolled in a study with active drug therapy and reveals an increase of serum AT to >3×ULN or bilirubin >2×ULN should undergo detailed testing for liver enzymes (including at least ALP, ALT, AST, TBL, and INR). Testing should be repeated within 72 hours of notification of the test results. For studies for which a central laboratory is used, alerts will be generated by the central laboratory regarding moderate and severe liver abnormality to inform the investigator and study team. Participants should be asked if they have any symptoms suggestive of hepatobiliary dysfunction.

### Definition of Liver Abnormalities

Confirmed abnormalities will be characterized as moderate and severe where ULN is as shown in Table 7, below.

TABLE 7
Moderate and Severe Liver Abnormalities

| | ALT or AST | | TBL |
|---|---|---|---|
| Moderate | >3 × ULN | or | >2 × ULN |
| Severe | >3 × ULN | and† | >2 × ULN |

ALT: alanine aminotransferase;
AST: aspartate aminotransferase;
TBL: total bilirubin;
ULN: upper limit of normal
†Samples taken simultaneously or within maximum 24 hours.

In addition, the participant should be considered to have severe hepatic abnormalities if any of the following apply:
ALT or AST >8×ULN
ALT or AST >5×ULN for more than 2 weeks
ALT or AST >3×ULN and† TBL >2×ULN or INR >1.5 (if INR testing is applicable/evaluated)
ALT or AST >5×ULN and† (TBL >2×ULN in participants with liver metastases)
ALT or AST >3×ULN with the appearance of fatigue, nausea, vomiting, right upper quadrant pain or tenderness, fever, rash and/or eosinophilia (>5%)

The investigator may determine that abnormal liver function results, other than as described above, may qualify as moderate or severe abnormalities and require additional monitoring and follow-up.

### Follow-Up Procedures

Confirmed moderate or severe abnormalities in hepatic functions should be thoroughly characterized by obtaining appropriate expert consultations, detailed pertinent history, physical examination and clinical laboratory tests. The study site personnel are to complete the LA-CRF. Participants with confirmed abnormal liver function testing should be followed as described below.

Confirmed moderately abnormal liver function tests should be repeated 2 to 3 times weekly, and then weekly or less frequently if abnormalities stabilize or the study intervention has been discontinued and the participant is asymptomatic.

Severe hepatic liver function abnormalities as defined above, in the absence of another etiology, may be considered an important medical event and may be reported as a SAE. The sponsor should be contacted and informed of all participants for whom severe hepatic liver function abnormalities possibly attributable to study intervention are observed.

To further assess abnormal hepatic laboratory findings, the investigator is expected to:
Obtain a more detailed history of symptoms and prior or concurrent diseases. Symptoms and new-onset diseases are to be recorded as “AEs” within the eCRF. Illnesses and conditions such as hypotensive events, and decompensated cardiac disease that may lead to secondary liver abnormalities should be noted. NASH is seen in obese hyperlipoproteinemic and/or diabetic participants, and may be associated with fluctuating AT levels. The investigator should ensure that the medical history form captures any illness that predates study enrollment that may be relevant in assessing hepatic function.
Obtain a history of concomitant drug use (including nonprescription medication, complementary and alternative medications), alcohol use, recreational drug use and special diets. Medications are to be entered in the eCRF. Information on alcohol, other substance use and diet should be entered on the LA-CRF or an appropriate document.
Obtain a history of exposure to environmental chemical agents.
Based on the participant's history, other testing may be appropriate including:
   Acute viral hepatitis (A, B, C, D, E or other infectious agents)
   Ultrasound or other imaging to assess biliary tract disease
   Other clinical laboratory tests, including INR and direct bilirubin
Consider gastroenterology or hepatology consultations.
Submit results for any additional testing and possible etiology on the LA-CRF or an appropriate document.

### Study Intervention Discontinuation

In the absence of an explanation for increased liver function tests, such as viral hepatitis, pre-existing or acute liver disease, or exposure to other agents associated with liver injury, the participant may be discontinued from study intervention. The investigator may determine that it is not in the participant's best interest to continue study intervention. Discontinuation of study intervention should be considered if:
ALT or AST >8×ULN
ALT or AST >5×ULN for more than 2 weeks
ALT or AST >3×ULN and† TBL >2×ULN or INR >1.5 (if INR testing is applicable/evaluated)
ALT or AST >5×ULN and† (TBL >2×ULN in participants with liver metastases)
ALT or AST >3×ULN with the appearance of fatigue, nausea, vomiting, right upper quadrant pain or tenderness, fever, rash and/or eosinophilia (>5%)
† Samples taken simultaneously or within a maximum of 24 hours.

In addition, if close monitoring for a participant with moderate or severe hepatic laboratory tests is not possible, study intervention should be discontinued.

Drug-induced jaundice caused by hepatocellular injury, without a significant obstructive component, has a high rate of bad outcomes, from 10% to 50% mortality (or transplant).

The 3 “requirements” for Hy's Law are:
1. Evidence that a drug can cause hepatocellular-type injury, generally shown by an increase in AT elevations >3×ULN (“2×ULN elevations are too common in treated and untreated participants to be discriminating”).
2. Cases of increased TBL (at least 2×ULN) with concurrent AT elevations at least 3×ULN and no evidence of intra- or extra-hepatic bilirubin obstruction (elevated ALP) or Gilbert's syndrome [Temple, 2006].
3. No other reason can be found to explain the combination of increased AT and TBL, such as viral hepatitis A, B, or C; pre-existing or acute liver disease; or another drug capable of causing the observed injury.

### Appendix 5: Clinical Laboratory Assessments

Laboratory tests, as shown in Table 8, below, will be performed according to the Schedule of Assessments (FIG. 4) and sent to the local laboratory for analysis.

TABLE 8
Clinical Laboratory Tests

| Panel/Assessments | Parameters to be Analyzed |
|---|---|
| Hematology | Hematocrit |
| | Hemoglobin |
| | Mean corpuscular volume |
| | Mean corpuscular hemoglobin |
| | Mean corpuscular hemoglobin concentration |
| | Platelets |
| | Red blood cell count |
| | White blood cell count |
| | White blood cell count differential (include |
| | differential specifics as needed) |
| Biochemistry | Albumin |
| | Alanine aminotransferase |
| | Alkaline phosphatase |
| | Aspartate aminotransferase |
| | Bicarbonate |
| | Blood urea nitrogen |
| | Chloride |
| | Creatinine |
| | Creatine kinase |
| | Glucose |
| | Lactate dehydrogenase |
| | Magnesium |
| | Phosphate |
| | Potassium |
| | Serum hCG for female participants of |
| | childbearing potential |
| | Serum ionized calcium |
| | Sodium |
| | Total and direct bilirubin |
| | Total protein |
| Serology | anti-HBS |
| | HAV (IgM) |
| | HbsAg |
| | HBV DNA (if isolated presence of anti-HBc in |
| | the absence of HbsAg and anti-HBs) |
| | HCV |
| | IgG anti-HBC |
| | IgM anti-HBC |
| Urinalysis | Bilirubin |
| | Glucose |
| | Ketones |
| | pH |
| | Protein |
| | Red blood cells |
| | Urine pregancy test |
| | Urobilinogen |
| Coagulation | Activated partial thromboplastin time |
| | International normalized ratio |
| | Prothrombin time (sec) |
| Thyroid | TSH (thyroid stimulating hormone) |
| | Free T4 (thyroxine) |
| | Free T3 or total T3 (triiodothyronine) |

anti-HBs: hepatitis B surface antibody;
HAV: hepatitis A virus;
HbsAg: hepatitis B surface antigen;
HBV DNA: hepatitis B virus DNA;
HCV: hepatitis C virus;
IgG anti-HBC: immunoglobulin G antibody to hepatitis B core antigen;
IgM anti-HBc: immunoglobulin M antibody to hepatitis B core antigen;
T3: triiodothyronine;
T4: thyroxine;
TSH: thyroid stimulating hormone

### Appendix 6: Pharmacogenomic Analysis with Banked Sample

### Introduction

PGx research aims to provide information regarding how naturally-occurring differences in a participant's gene and/or expression of genes based on genetic variation may impact what treatment options are best suited for the participant. Through investigation of PGx by technologies such as genotyping, gene sequencing, statistical genetics and Genome-Wide Association studies, the relationship between gene profiles and an intervention's kinetics, efficacy, toxicity or disease may be better understood. As many diseases may be influenced by 1 or more genetic variations, PGx research may identify which genes are involved in determining the way a participant may or may not respond to a drug.

### Objectives

The PGx research that may be conducted in the future with acquired blood samples is exploratory. The objective of this research will be to analyze or determine genes of relevance to clinical response, pharmacokinetics and/or toxicity/safety and/or disease.

By analyzing genetic variations, it may be possible to predict an individual participant's response to treatment in terms of efficacy and/or toxicity and/or disease.

### Participant Participation

Participants who have consented to participate in this study will participate in the PGx substudy. Participants must provide written consent prior to providing any blood samples that may be used at a later time for PGx analysis.

### Sample Collection and Storage

Participants who consent to participate in this substudy will provide a 4 mL sample of whole blood. Each sample will be identified by the unique participant number. Samples will be shipped to a designated banking CRO as directed by the sponsor.

### PGx Analysis

Details on the potential PGx analysis cannot be established yet. The sponsor may initiate the PGx analysis if evidence suggests that genetic variants may be influencing the intervention's pharmacokinetics, efficacy and/or safety and/or disease.

### Disposal of PGx Samples/Data

All PGx samples collected will be stored for a period of up to 15 years following study database lock. If there is no requirement for analysis, the whole blood sample will be destroyed after the planned storage period. The participant has the right to withdraw consent at any time. When a participant's withdrawal notification is received, the PGx sample will be destroyed. The results of any PGx analysis conducted on a sample prior to the participant's withdrawal will be retained by the sponsor indefinitely unless otherwise specified by local regulation.

### Information Disclosure to the Participants

Exploratory PGx analysis may be conducted following the conclusion of the study, if applicable. The results of the PGx analysis will not be provided to any investigators or participants, nor can the results be requested at a later date. Any information that is obtained from the PGx analysis will be the property of the sponsor.

### Appendix 7: Clinical Study Continuity

### Introduction

The purpose of this appendix is to provide acceptable alternate methods to assess safety and efficacy parameters, as appropriate, in the event the clinical study is interrupted at the country, state, site or participant level during any crisis (e.g., natural disaster, pandemic).

### Benefit-Risk Rationale

Maintaining the safety of clinical study participants and delivering continuity of care in the clinical study setting is paramount during any crisis. The site is expected to follow the protocol and associated Schedule of Assessments (FIG. 4) unless the site principal investigator discusses the need with the medical monitor to implement the alternate measures.

The approach outlined within this appendix defines which assessments are required to maintain a favorable benefit/risk to the participant, to maintain overall study integrity and to provide acceptable alternate methods to complete the study-required assessments and procedures if study activities are unable to be performed as described in the Study Procedures and Assessments section, above, due to a crisis.

### Informed Consent

Participants who need to follow any or all of the alternate measures outlined in this appendix will be required to provide informed consent, which explicitly informs them of the nature of and rationale for these changes, and gain their agreement to continue participation in the study prior to the implementation of any of these changes. In the event the urgency of implementing the alternate measures does not allow for the participant to provide written consent prior to implementation, the principal investigator or designee will obtain oral agreement from the participant followed by written documentation as soon as is feasible. A separate addendum to the study ICF will be provided to document the participant's consent to the changes.

### Participant Procedures Assessment

Sites with participants who are currently enrolled into this clinical study may consider implementing the alternate methods outlined below if 1 or more of the following conditions are met due to the crisis:
Regional or local travel has been restricted, inclusive of mandatory shelter in place measures, which makes participant travel to/from the study site nearly impossible.
Site facilities have been closed for clinical study conduct.
Site has been restricted to treating patients with conditions outside of the scope of the study.
Site personnel have temporarily relocated the conduct of the study to a location that place a burden on the participant with respect to time and travel.
Participant(s) have temporarily relocated from the current study site to an alternate study site to avoid placing a burden on the participant with respect to travel.
Participant(s) have temporarily relocated from their home location and the new distances from the site would cause undue burden with respect to time and travel.
Participant has risk factors for which traveling to the site poses an additional risk to the participant's health and safety.

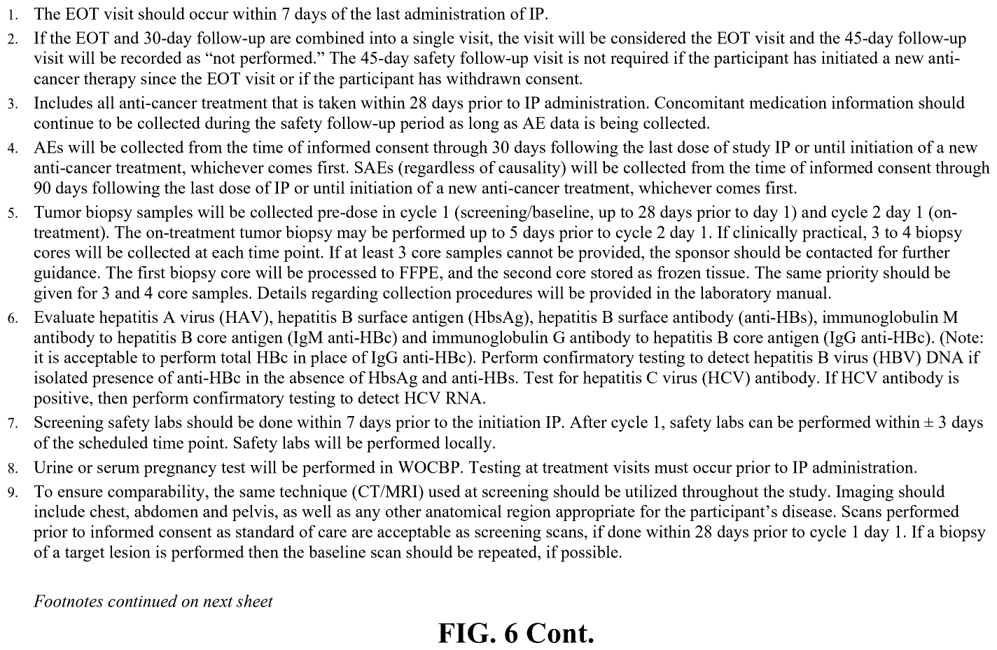

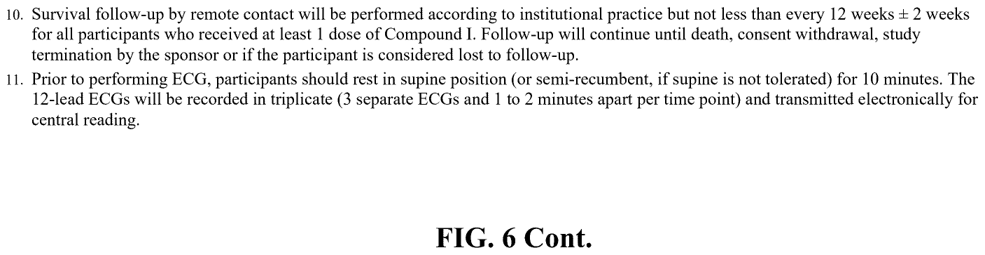

Adherence to the original protocol as reflected in the Schedule of Assessments (FIG. 4) is expected, where plausible, in the case of a crisis. The alternate measures as noted in FIG. 6 are only permissible in the event of a crisis, and after discussing the need with the medical monitor to implement the alternate measures. This is to allow for continuity of receiving IP and maintaining critical safety and efficacy assessments for participants in the study at a time of crisis.

If one or more of the alternate measures noted below is implemented for a participant, the site should document in the participant's source document the justification for implementing the alternate measure and the actual alternate measures that were implemented, along with the corresponding time point(s).

### Study Intervention Supply

If any of the conditions outlined above in the Participants Procedures Assessment are met, 1 or all of the following mitigating strategies will be employed, as needed, to ensure continuity of study intervention supply to the participants:
Increase stock of study intervention on site to reduce number of shipments required, if site space will allow.
Direct-to-Participant (DTP) shipments of study intervention from the site to the participant's home.

### Data Collection Requirements

Additional data may be collected in order to indicate how participation in the study may have been affected by a crisis and to accommodate data collection resulting from alternate measures implemented to manage the conduct of the study and participant safety.
Critical assessments for safety and efficacy based on study endpoints to be identified as missing or altered (performed virtually, at alternative locations, out of window or other modifications) due to the crisis.

### Appendix 8: List of Abbreviations and Definition of Key Study Terms

### List of Abbreviations

| Abbreviations | Description of abbreviations |
|---|---|
| ADL | activities of daily living |
| AE | adverse event |
| ALP | alkaline phosphatase |
| ALT | alanine aminotransferase |
| ANCOVA | analysis of covariance |
| AST | aspartate aminotransferase |
| AUC | area under the concentration-time curve |
| AxMP | auxiliary medicinal product |
| BMI | body mass index |
| BOP2 | 115ayesian optimal phase 2 |
| BSA | body surface area |
| BUN | blood urea nitrogen |
| CA | competent authority |
| CCDS | company core data sheet |
| CCSI | company core safety information |
| CDISC | Clinical Data Interchange Standards Consortium |
| CIOMS | Council for International Organizations |
| | of Medical Sciences |
| Cₘₐₓ | maximum concentration |
| CNS | central nervous system |
| COA | clinical outcomes assessment |
| CRF | case report form |
| CRO | contract research organization |
| CT | computed tomography |
| CTCAE | common terminology criteria for adverse events |
| CTIS | clinical trials information system |
| DBP | diastolic blood pressure |
| DCR | disease control rate |
| DGK | diacylglycerol kinase |
| DILI | drug-induced liver injury |
| DLT | dose-limiting toxicity |
| DMPA | depot medroxyprogesterone acetate |
| DOR | duration of response |
| DPD | data Protection Directive |
| ECE | emergency code envelope |
| ECG | electrocardiogram |
| eCOA | electronic clinical outcome assessment |
| eCRF | electronic case report form |
| FAS | full analysis set |
| FSH | follicle stimulating hormone |
| GCP | Good Clinical Practice |
| GMP | Good Manufacturing Practice |
| GGT | gamma-glutamyltransferase |
| GPF | Global Protocol Format |
| HAV | hepatitis A virus |
| HBc | hepatitis B |
| HBsAg | hepatitis B surface antigen |
| hCG | human chorionic gonadotropin |
| HCV | hepatitis C virus |
| HIV | human immunodeficiency virus |
| HIPAA | Health Insurance Portability and Accountability Act |
| HRT | hormone replacement therapy |
| IB | Investigator's Brochure |
| ICF | informed consent form |
| ICH | International Council for Harmonisation of |
| | Technical Requirements for |
| | Pharmaceuticals for Human Use |
| IDE | Investigational Device Exemption |
| IDMC | independent data monitoring committee |
| IEC | Independent Ethics Committee |
| ILD | interstitial lung disease |
| IM | intramuscular |
| IMPD | investigational medicinal product dossier |
| IND | Investigational New Drug |
| INR | international normalized ratio |
| IRB | Institutional Review Board |
| iRECIST | Immune Response Evaluation Criteria in Solid Tumors |
| IRT | interactive response technology |
| ISN | international study number |
| IUD | intrauterine device |
| IUS | intrauterine system |
| LA-CRF | liver abnormality case report form |
| LAR | legally authorized representative |
| LS | least squares |
| LSLV | last subject last visit |
| MAD | multiple ascending dose |
| MRI | magnetic resonance imaging |
| MTD | maximum tolerated dose |
| NASH | nonalcoholic steatohepatitis |
| NCI CTCAE | U.S. National Cancer Institute common terminology |
| | criteria for adverse |
| | events |
| NIMP | noninvestigational medicinal product |
| NIS | Noninterventional studies |
| ORR | objective response rate |
| OS | overall survival |
| PAC | protocol approval committee |
| PD-L1 | programmed death-ligand 1 |
| PFS | progression free survival |
| PGx | pharmacogenomic |
| PIP | Pediatric Investigation Plan |
| PKAS | pharmacokinetic analysis set |
| PPS | per protocol set |
| PT | preferred term |
| QA | quality assurance |
| QC | quality control |
| QTcF | corrected QT interval by Fredericia |
| QTL | quality tolerance limit |
| RBC | red blood cell |
| RECIST | Response Evaluation Criteria in Solid Tumors |
| RSI | reference safety information |
| SAD | single ascending dose |
| (S)AE | serious adverse event or adverse event |
| SAE | serious adverse event |
| SAP | statistical analysis plan |
| SBP | systolic blood pressure |
| SC | subcutaneous |
| SOC | system organ class |
| SOP | standard operating procedure |
| SmPC | summary of product characteristics |
| SRC | Scientific Review Committee |
| SUSAR | suspected unexpected serious adverse reactions |
| t_{1/2} | terminal elimination half-life |
| TEAE | treatment-emergent adverse event |
| TBL | total bilirubin |
| TIL | tumor-infiltrating lymphocytes |
| tₘₐₓ | time of maximum concentration |
| TME | tumor microenvironment |
| ULN | upper limit of normal |
| USM | urgent safety measure |
| WBC | white blood cell |
| WOCBP | woman/women of childbearing potential |

### Definition of Key Study Terms

| Terms | Definition of Terms |
|---|---|
| Baseline | Assessments of participants as they enter a study before they receive |
| | any treatment. |
| Endpoint | Variable that pertains to the efficacy or safety evaluations of a study. |
| | Note: Not all endpoints are themselves assessments since certain |
| | endpoints might apply to populations or emerge from analysis of |
| | results. That is, endpoints might be facts about assessments (e.g., |
| | prolongation of survival). |
| Enroll | To register or enter a participant into a study. Note: Once a participant |
| | has received the IP or placebo, the protocol applies to the participant. |
| Investigational | The drug, device, therapy or process under investigation in a study |
| Product | that is believed to have an effect on outcomes of interest in a study |
| | (e.g., health-related quality of life, efficacy, safety and |
| | pharmacogenomics). |
| Investigational | Period of time where major interests of protocol objectives are |
| period | observed, and where the test product or comparative drug (sometimes |
| | without randomization) is given to a participant, and continues until |
| | the last assessment after completing administration of the test product |
| | or comparative drug. |
| Post | Period of time after the last assessment of the protocol. Follow-up |
| investigational | observations for sustained adverse events and/or survival are done in |
| period | this period. |
| Randomization | The process of assigning participants to treatment or control groups |
| | using an element of chance to determine assignments in order to |
| | reduce bias. |
| | NOTE: Unequal randomization is used to allocate participants into |
| | groups at a differential rate; for example, 3 participants may be |
| | assigned to a treatment group for every one assigned to the control |
| | group. |
| Screening | A process of active consideration of potential participants for |
| | enrollment in a study. |
| Screen failure | Potential participant who signed the ICF, but did not meet one or |
| | more criteria required for participation in the study and was not |
| | enrolled. |
| Screening period | Period of time before entering the investigational period, usually from |
| | the time when a participant signs the consent form until just before the |
| | test product or comparative drug (sometimes without randomization) |
| | is given to a participant. |
| Study period | Period of time from the first study site initiation date to the last study |
| | site completing the study. |
| Variable | Any quantity that varies; any attribute, phenomenon or event that can |
| | have different qualitative or quantitative values. |

### Appendix 9: Monitoring Guidelines for Potential Immune-Related Adverse Events

### Symptoms of Potential Immune-Related Adverse Events

| Potential irAE | Symptoms |
|---|---|
| Pneumonitis | New cough, worsening cough, shortness of breath or chest |
| | pain |
| Colitis | Changes in bowel habits; abdominal pain; blood or mucus |
| | in stool; nausea |
| Hepatitis | Yellowing of skin or whites of eyes; pain on right side of |
| | abdomen; dark urine (color of tea); nausea or vomiting; |
| | bleeding or bruising more easily than usual; loss of appetite; |
| | drowsiness |
| Endocrinopathies | Persistent or unusual headaches, changes in vision, rapid |
| | heartbeat, increased sweating, feeling very tired or weak, |
| | achy muscles, change in weight (gain or loss), feeling |
| | lightheaded or feeling faint, feeling more hungry or thirsty |
| | than usual, loss of hair, mood changes such as reduced sex |
| | drive or increased irritability, forgetfulness, feeling cold, |
| | constipation, deeper voice, urinating more frequently than |
| | usual, nausea or vomiting, abdominal pain |
| Motor/sensory neuropathy; | Numbness or tingling, weakness, confusion, headache, |
| Encephalitis; Myasthenic | forgetfulness, changes in mood or behavior, fever, increased |
| syndrome/myasthenic | sensitivity to light, neck stiffness |
| gravis or Guillain-Barre | |
| syndrome | |
| Ocular Inflammation | Changes in vision (blurry vision; double vision; other vision |
| | changes), eye pain, eye redness, eyelid swelling |
| Pancreatitis | Nausea or vomiting, abdominal pain |
| Infection | Fever, other signs of infection |
| Musculoskeletal | New or worsening joint symptoms, muscle weakness or |
| inflammation | pain |

irAE: immune-related adverse event
Note:
These events have not been observed with Compound I but have been observed in clinical trials with other immuno-oncology agents such as immune checkpoint inhibitors.

### Recommended Guidelines for Management of Immune-Related Adverse Events

| | Toxicity | |
|---|---|---|
| | Grade | |
| | (CTCAE | |
| irAEs | v5.0) | Recommended Guidelines for Management‡‡ |
| Pneumonitis | Grade 2 | Withhold Compound I until toxicity improves |
| | | to ≤grade 1 |
| | | Corticosteroids (initial dose of 0.5 to 1 mg/kg per |
| | | day of prednisone or equivalent) may be |
| | | administered. |
| | | Taper corticosteroids over 4 to 6 weeks. |
| | Recurrent | Permanently discontinue |
| | Grade 2, | Administer high-dose corticosteroids (prednisone |
| | Grade 3 or 4 | 1 to 2 mg/kg per day or methylprednisolone |
| | | intravenous 1 to 2 mg/kg per day) until toxicity |
| | | improves to ≤grade 1. |
| | | Taper corticosteroids over 4 to 6 weeks. |
| Diarrhea/Colitis | Grade 2 | Withhold Compound I until toxicity improves |
| | Grade 3 | to ≤grade 1 |
| | | Corticosteroids (initial dose of 0.5 to 1 mg/kg per |
| | | day of prednisone or equivalent) may be |
| | | administered. |
| | | Taper corticosteroids over 4 to 6 weeks. |
| | | Withhold Compound I until toxicity improves |
| | | to ≤grade 1. |
| | | Administer high-dose corticosteroids (prednisone |
| | | 1 to 2 mg/kg per day or methylprednisolone |
| | | intravenous 1 to 2 mg/kg per day). |
| | | If symptoms do not improve with 48 to 72 hours |
| | | of high-dose corticosteroids, infliximab may be |
| | | administered. |
| | | Taper corticosteroids over 4 to 6 weeks. |
| | Recurrent | Stop Compound I immediately and permanently |
| | Grade 3 or | discontinue the subject. |
| | Grade 4 | Administer high-dose corticosteroids (prednisone |
| | | 1 to 2 mg/kg per day or methylprednisolone |
| | | intravenous 1 to 2 mg/kg per day) until toxicity |
| | | improves to ≤grade 1. |
| | | Taper corticosteroids over 4 to 6 weeks. |
| AST or ALT | Grade 2‡ | Withhold Compound I until toxicity improves |
| elevation or | | to ≤grade 1 |
| Increased | | Corticosteroids (initial dose of 0.5 to 1 mg/kg per |
| Bilirubin | | day of prednisone or equivalent) may be |
| | | administered. |
| | | Taper corticosteroids over 4 to 6 weeks. |
| | Grade 3§ or 4¶ | Stop Compound I immediately and |
| | | permanently discontinue the subject. |
| | | Administer high-dose corticosteroids |
| | | (prednisone 1 to 2 mg/kg per day or |
| | | methylprednisolone intravenous 1 to 2 mg/kg |
| | | per day) until toxicity improves to ≤grade 1. |
| | | Taper corticosteroids over 4 to 6 weeks. |
| Type 1 diabetes | New onset | Stop Compound I immediately and permanently |
| mellitus | T1DM or | discontinue the subject. † |
| (T1DM) or | Grade 3 or 4 | Administer high-dose corticosteroids (prednisone |
| Hyperglycemia | hyperglycemia | 1 to 2 mg/kg per day or methylprednisolone |
| | associated | intravenous 1 to 2 mg/kg per day) until toxicity |
| | with evidence | improves to ≤grade 1. |
| | of β-cell | Taper corticosteroids over 4 to 6 weeks. |
| | failure | |
| Endocrine Other | Grade 2 | Withhold Compound I until toxicity improves |
| (Hypophysitis) | | to ≤grade 1. |
| | | Corticosteroids (initial dose of 0.5 to 1 mg/kg per |
| | | day of prednisone or equivalent) may be |
| | | administered. |
| | | Taper corticosteroids over 4 to 6 weeks. |
| | Grade 3 or 4 | Stop Compound I immediately and withhold or |
| | | permanently discontinue the subject. † |
| | | Administer high-dose corticosteroids (prednisone |
| | | 1 to 2 mg/kg per day or methylprednisolone |
| | | intravenous 1 to 2 mg/kg per day) until toxicity |
| | | improves to ≤grade 1. |
| | | Taper corticosteroids over 4 to 6 weeks. |
| Hyperthyroidism | Grade 2 | Continue |
| | Grade 3 or 4 | Stop Compound I immediately and withhold or |
| | | permanently discontinue the subject. † |
| | | Administer high-dose corticosteroids (prednisone |
| | | 1 to 2 mg/kg per day or methylprednisolone |
| | | intravenous 1 to 2 mg/kg per day) until toxicity |
| | | improves to ≤grade 1. |
| | | Taper corticosteroids over 4 to 6 weeks. |
| Hypothyroidism | Grade 2, 3, 4 | Continue |
| Nephritis: | Grade 2 | Withhold Compound I until toxicity improves to ≤ |
| grading | | grade 1. |
| according to | | Corticosteroids (initial dose of 0.5 to 1 mg/kg per |
| increased | | day of prednisone or equivalent) may be |
| creatinine or | | administered. |
| acute kidney | | Taper corticosteroids over 4 to 6 weeks. |
| injury | Grade 3 or 4 | Stop Compound I immediately and permanently |
| | | discontinue the subject. |
| | | Administer high-dose corticosteroids (prednisone |
| | | 1 to 2 mg/kg per day or methylprednisolone |
| | | intravenous 1 to 2 mg/kg per day) until toxicity |
| | | improves to ≤grade 1. |
| | | Taper corticosteroids over 4 to 6 weeks. |
| Myocarditis | Grade 1 or 2 | Withhold Compound I until toxicity improves |
| | | to ≤grade 1. |
| | | Corticosteroids (initial dose of 0.5 to 1 mg/kg per |
| | | day of prednisone or equivalent) may be |
| | | administered. |
| | | Taper corticosteroids over 4 to 6 weeks. |
| | Grade 3 or 4 | Stop Compound I immediately and permanently |
| | | discontinue the subject. |
| | | Administer high-dose corticosteroids (prednisone |
| | | 1 to 2 mg/kg per day or methylprednisolone |
| | | intravenous 1 to 2 mg/kg per day) until toxicity |
| | | improves to ≤ grade 1. |
| | | Taper corticosteroids over 4 to 6 weeks. |
| All Other | Persistent | Withhold Compound I until toxicity improves |
| immune-related | Grade 2 | to ≤grade 1. |
| AEs | | Corticosteroids (initial dose of 0.5 to 1 mg/kg per |
| | | day of prednisone or equivalent) may be |
| | | administered. |
| | | Taper corticosteroids over 4 to 6 weeks. |
| | Grade 3 | Withhold or discontinue based on the event†† |
| | Recurrent | Permanently discontinue |
| | Grade 3 or | |
| | Grade 4 | |

AE: adverse event; ALT: alanine aminotransferase; AST: aspartate aminotransferase; GI: gastrointestinal; irAE: immune-related adverse events; T1DM: Type 1 diabetes mellitus.
† Subjects with controlled endocrinopathies may resume Compound I under the discretion of the investigator. If control achieved or ≤ grade 2, Compound I may be resumed.
‡AST/ALT: >3.0 to 5.0 × ULN if baseline normal; >3.0 to 5.0 × baseline, if baseline abnormal; bilirubin: >1.5 to 3.0 × ULN if baseline normal; >1.5 to 3.0 × baseline if baseline abnormal.
§AST/ALT: >5.0 to 20.0 × ULN, if baseline normal; >5.0 to 20.0 × baseline, if baseline abnormal; bilirubin: >3.0 to 10.0 × ULN if baseline normal; >3.0 to 10.0 × baseline if baseline abnormal.
AST/ALT: >20.0 × ULN, if baseline normal; >20.0 × baseline, if baseline abnormal; bilirubin: >10.0 × ULN if baseline normal; >10.0 × baseline if baseline abnormal.
††Events that require discontinuation include but are not limited to: Guillain-Barre Syndrome, encephalitis, Stevens-Johnson Syndrome and toxic epidermal necrolysis.
‡‡Recommended guidelines to be considered in conjunction with institutional guidelines and standard of care, if clinically indicated and appropriate Source: adapted from Brahmer JR, et al. Management of immune-related adverse events in patients treated with immune checkpoint inhibitor therapy: American Society of Clinical Oncology clinical practice guideline. J Clin Oncol 2018; 36(17): 1714-68

### Appendix 10: Algorithm for Management of Cytokine Release Syndrome

| | Potential Clinical Signs and | |
|---|---|---|
| CRS Stage | Symptoms | Intervention |
| Prodrome | Low grade fever, fatigue, | Admit for observation |
| (onset hours to | myalgia, anorexia, flu-like | Infection work-up |
| days) | symptoms | Antibiotics per institutional |
| | | guidelines |
| | | Acetaminophen or NSAID as needed |
| Grade 1 | Fever ≥38° C. not | O₂ nasal canula |
| | requiring vasopressors or | iv fluids |
| | O₂ support | Anti-pyretic |
| | | Monitor for/manage TLS |
| | | Consider tocilizumab for prolonged |
| | | (>3 days) grade 1 CRS in |
| | | participants with significant |
| | | symptoms and/or comorbidities, |
| | | consider 1 dose of tocilizumab 8 |
| | | mg/kg over 1 hour (not to exceed |
| | | 800 mg) |
| | | Symptomatic management of organ |
| | | toxicities |
| Grade 2 | Hemodynamic instability may | Tocilizumab 8 mg/kg iv over 1 hour, |
| | require iv fluid support, but | max dose 800 mg repeat in 8 hours |
| | does not require vasopressors, | if no improvement, no more than 3 |
| | worsening respiratory distress | doses in 24 hours, with a maximum |
| | that requires low-flow O₂ | of 4 doses total^{‡}. |
| | (≤6 L/minute) nasal canula or | O₂ respiratory support as needed |
| | blow-by | iv fluid bolus as needed |
| | | Consider steroids or other anti- |
| | | cytokine therapies (e.g., |
| | | siltuximab, anakinra, |
| | | canakinumab) |
| | | Rule out sepsis |
| | | Symptomatic management of organ |
| | | toxicities |
| Grade 3 | Continued clinical | Second dose of tocilizumab 8 |
| | deterioration or lack of | mg/kg iv, max dose 800 mg, |
| | improvement after 1 dose of | administered every 8 hours up to 4 |
| | tocilizumab. Hypotension | doses^{‡}, consider steroids if no |
| | requires at least one | improvement within 24 hours |
| | vasopressor. Increasing O₂ | Steroids, dexamethasone 10 mg iv |
| | requirement or need for | every 6 hours^{†}, rapid taper if clinical |
| | mechanical ventilation, rapid | improvement |
| | clinical deterioration | Consider other anti-cytokine |
| | | therapies (e.g., siltuximab, |
| | | anakinra, canakinumab) |
| | | Vasopressor support as needed |
| | | Continue hemodynamic and |
| | | respiratory support |
| | | Symptomatic management of organ |
| | | toxicities |
| Grade 4 | Continued clinical deterioration | Tocilizumab therapy as per grade |
| | or lack of improvement despite | 2^{‡} if maximum dose not reached |
| | intervention with steroids and | within 24-hour period |
| | tocilizumab | Steroids^{‡}, dexamethasone 10 mg iv |
| | | every 6 hours. If refractory, |
| | | consider 3 doses of |
| | | methylprednisolone 1000 mg/day |
| | | iv; if refractory, consider dosing |
| | | every 12 hours^{†},§ |
| | | Consider other anti-cytokine |
| | | therapies (e.g., siltuximab, |
| | | anakinra, canakinumab) |
| | | Consider cyclophosphamide, |
| | | alemtuzumab, or other anti-T cell |
| | | therapies |
| | | Continue hemodynamic and |
| | | respiratory support |
| | | Symptomatic management of |
| | | organ toxicities |

Adapted from [Lee et al, 2019] and NCCN Guidelines, version 3.2021
CRS: cytokine release syndrome; iv: intravenous; max: maximum; NCCN: National Comprehensive Cancer Network; NSAID: nonsteroidal anti-inflammatory drug; O₂: oxygen; TLS: tumor lysis syndrome.
^{†}Alternative steroids at an equivalent dose may be considered.
^{‡}After each dose, assess the need for subsequent dosing.
§For example, methylprednisolone iv 1000 mg/day for 3 days, followed by rapid taper at 250 mg every 12 hours for 2 days, 125 mg every 12 hours for 2 days, and 60 mg every 12 hours for 2 days.

### Appendix 11: Algorithm for Management of Immune-Effector Cell-Associated Neurotoxicity Syndrome

| Treatment by | | | Additional Therapy |
|---|---|---|---|
| ICANS Grade | Grading Criteria† | No Concurrent CRS | if Concurrent CRS |
| Grade 1 | ICE score: 7 to 9 | Supportive care | Tocilizumab 8 |
| | Awakens | | mg/kg iv over 1 |
| | spontaneously | | hour (not to exceed |
| | | | 800 mg/dose)‡ |
| Grade 2 | ICE score: 3 to 6 | Supportive care | Anti-IL-6 therapy |
| | Awakens to voice | 1 dose of | as per grade 1‡ |
| | | dexamethasone 10 mg | Consider |
| | | iv and reassess. Can | transferring to ICU |
| | | be repeated every 6 to | if neurotoxicity is |
| | | 12 hours, if no | associated with >grade |
| | | improvement. | 2 CRS |
| Grade 3 | ICE score: 0 to 2 | ICU care is | Anti-IL-6 therapy as |
| | Awakens only to | recommended | per grade 1‡ |
| | tactile stimulus | Dexamethasone 10 mg | |
| | Any clinical seizure | iv every 6 hours or | |
| | focal or generalized | methylprednisolone, | |
| | that resolves rapidly | 1 mg/kg iv every | |
| | or nonconvulsive | 12 hours§ | |
| | seizures on EEG | Consider repeat | |
| | that resolve with | neuroimaging (CT or | |
| | intervention | MRI) every 2 to 3 days | |
| | Focal/local edema | if participant has | |
| | on neuroimaging | persistent ≥grade 3 | |
| | | neurotoxicity. | |
| | | Consider lumbar | |
| | | puncture | |
| | | If seizures or seizure | |
| | | like activity is present, | |
| | | anti-epileptic drugs are | |
| | | recommended | |
| Grade 4 | ICE score: 0 | ICU care, mechanical | Anti-IL-6 |
| | (participant is | ventilation for airway | therapy as per |
| | unarousable and | protection may be | grade 1‡ |
| | unable to perform | indicated | |
| | ICE) | High dose steroids§,¶ | |
| | Participant is | If seizures or seizure- | |
| | unarousable or | like activity is present, | |
| | requires vigorous or | anti-epileptic drugs are | |
| | repetitive tactile | recommended. Treat | |
| | stimuli to arouse. | convulsive status | |
| | Stupor or coma. | epilepticus per | |
| | Life-threatening | institutional guidelines | |
| | prolonged seizure | Consider repeat | |
| | (>5 min); or | neuroimaging (CT or | |
| | repetitive clinical or | MRI) every 2 to 3 days | |
| | electrical seizures | if participant has | |
| | without return to | persistent ≥grade 3 | |
| | baseline in between | neurotoxicity. | |
| | Deep focal motor | | |
| | weakness such as | | |
| | hemiparesis or | | |
| | paraparesis | | |
| | Diffuse cerebral | | |
| | edema on | | |
| | neuroimaging; | | |
| | decerebrate or | | |
| | decorticate | | |
| | posturing; or | | |
| | cranial nerve VI | | |
| | palsy; or | | |
| | papilledema; or | | |
| | Cushing's triad | | |

CRS: cytokine release syndrome; CT: computed tomography; EEG: electroencephalogram; ICANS: immune-effector cell-associated neurotoxicity syndrome; ICE: immune-effector cell-associated encephalopathy; ICU: intensive care unit; IL-6: interleukin-6; iv: intravenous; MRI: magnetic resonance imaging; NCCN: National Comprehensive Cancer Network.
†ICANS grade is determined by the most severe event (ICE score, level of consciousness, seizure, motor findings, raised ICP/cerebral edema) not attributable to any other cause for example, a participant with an ICE score of 3 who has a generalized seizure is classified as grade 3 ICANS.
‡Repeat tocilizumab every 8 hours as needed if not responsive to iv fluids or increasing supplemental oxygen. Limit to a maximum of 3 doses in a 24-hour period; maximum total of 4 doses.
§Antifungal prophylaxis should be strongly considered in participants receiving steroids for the treatment of CRS and/or neurotoxicity.
For example, methylprednisolone iv 1000 mg/day (may consider twice a day) for 3 days, followed by rapid taper at 250 mg every 12 hours for 2 days, 125 mg every 12 hours for 2 days, and 60 mg every 12 hours for 2 days.
Source: NCCN Guidelines Version 3.2021, Management of Immunotherapy-Related Toxicities. May 2021.

### REFERENCES

American Cancer Society. Cancer facts & figures 2020. Atlanta: American Cancer Society; 2020. Available from: https://www.cancer.org/content/dam/cancer-org/research/cancer-facts-and-statistics/annual-cancer-facts-and-figures/2020/cancer-facts-and-figures-2020.pdf Accessed on 23 Apr. 2021.
Lanitis E, Dangaj D, Irving M, Coukos G. Mechanisms regulating T-cell infiltration and activity in solid tumors. Ann Oncol. 2017; 28:xii18-32.
Lee D W, Santomasso B D, Locke F L, Ghobadi A, Turtle C J, Brudno I N, et al. ASTCT Consensus Grading for Cytokine Release Syndrome and Neurologic Toxicity Associated with Immune Effector Cells. Biol Blood Marrow Transplant 2019; 25:625-38.
Luke J J, Flaherty K T, Ribas A, Long G V. Targeted agents and immunotherapies: optimizing outcomes in melanoma. Nat Rev Clin Oncol. 2017; 14(8):463-82.
National Cancer Institute [Internet]. Cancer Statistics; 2018 [cited 2019 Apr. 16]. Available from: https://www.cancer.gov/about-cancer/understanding/statistics.
Oken M M, Creech R H, Tormey D C, Horton J, Davis T E, McFadden E T, et al. Toxicity and response criteria of the Eastern Cooperative Oncology Group. Am J Clin Oncol.
   1982; 5(6):649-55.
Temple R. Hy's Law: Predicting Serious Hepatotoxicity. Pharmacoepidemiol Drug Saf. 2006; 15(4):241-3.
Wee S, Gu J, Wang C, Cao C, Holzhauer S, Desilva H, et al. Abstract 936: Regulation of CD8+ T-cell function and antitumor activity by DGKα and DGKζ. American Association for Cancer Research (AACR) Annual Meeting 2019 (29 Mar. 3 Apr. 2019); Atlanta Georgia. Available at: https://cancerres.aacrjournals.org/content/79/13_Supplement/936. Accessed 10 May 2021.
Zhong X P, Guo R, Zhou H, Liu C, Wan C K. Diacylglycerol kinases in immune cell function and self-tolerance. Immunol Rev. 2008; 224:249-64.
Zhou H, Lee J J, Yuan Y. BOP2: Bayesian optimal design for phase II clinical trials with simple and complex endpoints. Statistics in Medicine. 2017; 36(21):3302-14.

1. A method for treating a patient having an advanced and/or metastatic solid tumor, the method comprising administering to the patient an effective amount of at least one entity chosen from Compound I 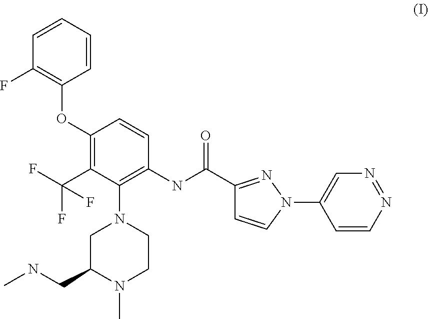 [SMILES: CNC[C@H]1CN(c2c(NC(=O)c3ccn(-c4ccnnc4)n3)ccc(Oc3ccccc3F)c2C(F)(F)F)CCN1C]
and pharmaceutically acceptable salts thereof.

2-44. (canceled)

45. The method according to claim 1 wherein the advanced and/or metastatic solid tumor is chosen from skin cancer, bladder cancer, breast cancer, uterine cancer, ovary cancer, prostate cancer, lung cancer, colon cancer, pancreas cancer, renal cancer, and gastric cancer.

46. The method according to claim 45, wherein the advanced and/or metastatic solid tumor is chosen from melanoma and non-small cell lung carcinoma.

47. The method according to claim 46, wherein the advanced and/or metastatic solid tumor is non-small cell lung carcinoma.

48. The method according to claim 1, wherein the patient satisfies at least one of the following conditions:
(a) the patient is considered an adult according to local regulation at the time of treatment;
(b) the patient has locally advanced or metastatic solid tumor malignancy, wherein the malignancy is confirmed by available pathology records or current biopsy;
(c) the patient has at least 1 measurable lesion per Response Evaluation Criteria in Solid Tumors v1.1, wherein lesions situated in a previously irradiated area are considered measurable if progression has been demonstrated in such lesions;
(d) the patient has progressed after receiving all standard approved therapies and/or is no longer eligible for standard therapy;
(e) the patient has an Eastern Cooperative Oncology Group Performance Status of 0, 1, or 2;
(f) the patient's last dose of any prior antineoplastic therapy, including any immunotherapy, was at least 21 days prior to being treated by the method; however, for the patient with solid tumors that have a neurotropic receptor tyrosine kinase gene fusion without a known acquired resistance mutation or for the patient with epidermal growth factor receptor or anaplastic lymphomas kinase mutation-positive non-small cell lung cancer, prior neurotropic receptor tyrosine kinase inhibitor or epidermal growth factor receptor tyrosine kinase inhibitor or anaplastic lymphomas kinase inhibitor therapy is allowed until 4 days prior to being treated by the method;
(g) the patient who has received radiotherapy, including stereotactic radiosurgery, must have completed the radiotherapy at least 2 weeks prior to being treated by the method;
(h) the patient's adverse events (excluding alopecia) from a prior therapy has improved to grade 1 or baseline within 14 days prior to being treated by the method; however, the patient that has type 1 diabetes mellitus, an endocrinopathy stably maintained on appropriate replacement therapy, or a skin disorder that does not require systemic treatment are allowed;
(i) the patient has adequate organ function prior to treatment as indicated by the following laboratory values, wherein the laboratory values must be obtained 2 weeks after any blood transfusion:
(i) absolute neutrophil count levels 1500/μL;
(ii) Platelets levels ≥100,000/μL;
(iii) Hemoglobin levels ≥9 g/dL;
(iv) Creatinine levels either ≤upper limit of normal OR creatinine clearance 60 mL/min as calculated by Cockroft-Gault equation;
(v) Total bilirubin levels either ≤1.5× upper limit of normal; OR, for participants with Gilbert's syndrome, direct bilirubin ≤upper limit of normal and total bilirubin <3× upper limit of normal;
(vi) aspartate aminotransferase (serum glutamic oxaloacetic transaminase) and alanine aminotransferase (serum glutamic pyruvic transaminase) levels ≤2.5× upper limit of normal without liver metastases or ≤5× upper limit of normal if liver metastases are present;
(vii) Serum potassium levels ≥3.4 mEq/L;
(viii) Serum magnesium levels ≥1.7 mg/dL; and
(ix) Serum ionized calcium levels ≥4.7 mg/dL;
(j) the patient has activated partial thromboplastin time and international normalized ratio ≤1.5× upper limit of normal and is not receiving anticoagulation;
(k) the patient, if female as assigned at birth, is not pregnant and at least one of the following conditions apply:
(i) the patient is not a woman of childbearing potential;
(ii) the patient is a woman of childbearing potential who agrees to follow contraceptive guidance while being treated by the method and for at least 30 days after end of treatment;
(l) the patient, if female as assigned at birth, agrees not to breastfeed during the period starting at screening, while being treated by the method, and for at least 30 days after end of treatment;
(m) the patient, if female as assigned at birth, does not donate ova while being treated by the method and for at least 30 days after end of treatment;
(n) the patient, if male as assigned at birth and has one or more female partners of childbearing potential (including breastfeeding partners), agrees to use contraception while being treated by the method and for at least 30 days after end of treatment;
(o) the patient, if male as assigned at birth, does not donate sperm while being treated by the method and for at least 30 days after end of treatment;
(p) the patient, if male as assigned at birth and has one or more pregnant partners, agrees to remain abstinent or use a condom for the duration of the pregnancy while being treated by the method and for at least 30 days after end of treatment;
(q) the patient agrees not to participate in another interventional study while being treated by the method.

49. The method according to claim 1, wherein the patient is excluded from treatment if any of the following conditions are satisfied:
(a) the patient has received any investigational therapy (other than possibly an epidermal growth factor receptor tyrosine kinase inhibitor used by a patient with epidermal growth factor receptor-activating mutations, an anaplastic lymphomas kinase inhibitor used by a patient with an anaplastic lymphomas kinase mutation or an neurotropic receptor tyrosine kinase inhibitor used by a patient with solid tumors that have a neurotropic receptor tyrosine kinase gene fusion without a known acquired resistance mutation) within 21 days or 5 half-lives, whichever is shorter, prior to being treated by the method;
(b) the patient requires or has received systemic steroid therapy or any other immunosuppressive therapy within 14 days prior to being treated by the method; however, patients using a physiologic replacement dose of hydrocortisone or its equivalent are allowed;
(c) the patient requires strong or moderate CYP2D6 inhibitors while being treated by the method;
(d) the patient requires strong CYP3A4 inhibitors while being treated by the method;
(e) the patient has symptomatic central nervous system metastases or evidence of unstable central nervous system metastases even if asymptomatic; however, patients with previously treated central nervous system metastases are eligible if they are clinically stable and have no evidence of central nervous system progression by imaging for at least 4 weeks prior to being treated by the method and are not requiring immunosuppressive doses of systemic steroids for no longer than 2 weeks, wherein immunosuppressive doses of systemic steroids comprises >30 mg per day of hydrocortisone or >10 mg per day of prednisone or equivalent;
(f) the patient has an active autoimmune disease; however, patients with type 1 diabetes mellitus, endocrinopathies stably maintained on appropriate replacement therapy, or skin disorders not requiring systemic treatment are allowed;
(g) the patient was discontinued from prior immunomodulatory therapy due to a grade ≥3 toxicity that was mechanistically related to the agent;
(h) the patient is known to have HIV infection; however, patients with HIV has CD4+ T-cell counts ≥350 cells/μL and no history of AIDS-defining opportunistic infections within the past 6 months are eligible;
(i) the patient has any of the following per screening serology test:
(i) Hepatitis A virus antibodies;
(ii) Positive hepatitis B surface antigen or detectable hepatitis B DNA; however, patients with negative HBsAg, positive hepatitis B core antibody and negative hepatitis B surface antibody are eligible if hepatitis B DNA is undetectable;
(iii) Hepatitis C virus antibodies unless Hepatitis C virus RNA is undetectable
(j) the patient has received a live vaccine against infectious diseases within 28 days prior being treated by the method;
(k) the patient has a history of drug-induced pneumonitis or currently has pneumonitis or a prior history of interstitial lung disease or noninfectious pneumonitis requiring high-dose glucocorticoids, whether resolved or not;
(l) the patient has an infection requiring systemic therapy within 14 days prior to being treated by the method;
(m) the patient has received a prior allogenic bone marrow or solid organ transplant;
(n) the patient is expected to require another form of antineoplastic therapy while being treated by the method;
(o) the patient has had a myocardial infarction or unstable angina within 6 months prior to being treated by the method or currently has an uncontrolled illness that would limit compliance with treatment;
(p) the patient has inadequately controlled hypertension, wherein inadequately controlled hypertension comprises as a systolic blood pressure >150 and/or diastolic blood pressure >100 mmHg on antihypertensive medications;
(q) the patient has a corrected QT interval using Fridericia's formula >450 ms prior to being treated by the method;
(r) the patient has another malignancy requiring active therapy, except for locally curable malignancies;
(s) the patient has had a major surgical procedure and has not completely recovered within 28 days prior to being treated by the method;
(t) the patient has a history of bleeding diathesis;
(u) the patient requires the use of any anticoagulation therapy;
(v) the patient has been previously treated with a DGK inhibitor; or
(w) the patient has a known or suspected hypersensitivity to the at least one entity.

50. The method according to claim 1, wherein the at least one entity is in the form of a pharmaceutically acceptable solvate, mixed solvate, or complex.

51. The method according to claim 1, where the at least one entity is in the form of a non-crystalline solid.

52. The method according to claim 1, wherein the at least one entity is in the form of a crystalline solid.

53. The method according to claim 1, wherein the at least one entity is Compound I.

54. The method according to claim 1, wherein the at least one entity is chosen from pharmaceutically acceptable salts of Compound I.

55. The method according to claim 54, wherein the at least one entity is chosen from HCl, mesylate, succinate, L-malate, L-tartrate, and fumarate salts of Compound I.

56. The method according to claim 55, wherein the at least one entity is a succinate salt of Compound 1.

57. The method according to claim 56, wherein the succinate salt of Compound I is a mono-succinate salt of Compound I.

58. The method according to claim 55, wherein the at least one entity is a L-malate salt of Compound I.

59. The method according to claim 58, wherein the L-malate salt of Compound I is a mono-L-malate salt of Compound I.

60. The method according to claim 58, wherein the L-malate salt of Compound I is a hemi-L-malate salt of Compound I.

61. A method for treating a patient having an advanced and/or metastatic solid tumor, the method comprising administering to the patient a pharmaceutical composition comprising an effective amount of at least one entity chosen from Compound I 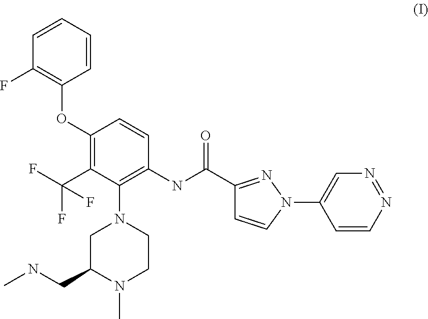 [SMILES: CNC[C@H]1CN(c2c(NC(=O)c3ccn(-c4ccnnc4)n3)ccc(Oc3ccccc3F)c2C(F)(F)F)CCN1C]
and pharmaceutically acceptable salts thereof.

62. The method according to claim 61, wherein the pharmaceutical composition comprises a dose equivalent of about 5 mg, about 10 mg, about 30 mg, about 60 mg, about 100 mg, about 150 mg, or about 200 mg Compound I.
